SEQUENCE LISTING (87 sequences)

SEQ ID NO: 1 - protein, 15 aa, synthetic construct
RAYNTSTGLALCYAS

SEQ ID NO: 2 - protein, 15 aa, synthetic construct
NAYNTSTGLALCYAS

SEQ ID NO: 3 - protein, 15 aa, synthetic construct
RVYNTSTGLALCYAS

SEQ ID NO: 4 - protein, 15 aa, synthetic construct
NVYNTSTGLALCYAS

SEQ ID NO: 5 - protein, 15 aa, synthetic construct
RAYNTSTGLALCYAN

SEQ ID NO: 6 - protein, 15 aa, synthetic construct
NAYNTSTGLALCYAN

SEQ ID NO: 7 - protein, 15 aa, synthetic construct
RVYNTSTGLALCYAN

SEQ ID NO: 8 - protein, 15 aa, synthetic construct
NVYNTSTGLALCYAN

SEQ ID NO: 9 - protein, 16 aa, synthetic construct
RAYNTSTGGLALCYAS

SEQ ID NO: 10 - protein, 16 aa, synthetic construct
NAYNTSTGGLALCYAS

SEQ ID NO: 11 - protein, 16 aa, synthetic construct
RVYNTSTGGLALCYAS

SEQ ID NO: 12 - protein, 16 aa, synthetic construct
NVYNTSTGGLALCYAS

SEQ ID NO: 13 - protein, 16 aa, synthetic construct
RAYNTSTGGLALCYAN

SEQ ID NO: 14 - protein, 16 aa, synthetic construct
NAYNTSTGGLALCYAN

SEQ ID NO: 15 - protein, 16 aa, synthetic construct
RVYNTSTGGLALCYAN

SEQ ID NO: 16 - protein, 16 aa, synthetic construct
NVYNTSTGGLALCYAN

SEQ ID NO: 17 - protein, 16 aa, synthetic construct
RAYNTSTCGLALCYAN

SEQ ID NO: 18 - protein, 16 aa, synthetic construct
NAYNTSTCGLALCYAN

SEQ ID NO: 19 - protein, 16 aa, synthetic construct
RVYNTSTCGLALCYAN

SEQ ID NO: 20 - protein, 16 aa, synthetic construct
NVYNTSTCGLALCYAN

SEQ ID NO: 21 - protein, 17 aa, synthetic construct
RAYNTSTCGGLALCYAN

SEQ ID NO: 22 - protein, 17 aa, synthetic construct
NAYNTSTCGGLALCYAN

SEQ ID NO: 23 - protein, 17 aa, synthetic construct
RVYNTSTCGGLALCYAN

SEQ ID NO: 24 - protein, 17 aa, synthetic construct
NVYNTSTCGGLALCYAN

SEQ ID NO: 25 - protein, 16 aa, synthetic construct
RAYNTSTCGLALCYAS

SEQ ID NO: 26 - protein, 16 aa, synthetic construct
NAYNTSTCGLALCYAS

SEQ ID NO: 27 - protein, 16 aa, synthetic construct
RVYNTSTCGLALCYAS

SEQ ID NO: 28 - protein, 16 aa, synthetic construct
NVYNTSTCGLALCYAS

SEQ ID NO: 29 - protein, 17 aa, synthetic construct
RAYNTSTCGGLALCYAS

SEQ ID NO: 30 - protein, 17 aa, synthetic construct
NAYNTSTCGGLALCYAS

SEQ ID NO: 31 - protein, 17 aa, synthetic construct
RVYNTSTCGGLALCYAS

SEQ ID NO: 32 - protein, 17 aa, synthetic construct
NVYNTSTCGGLALCYAS

SEQ ID NO: 33 - protein, 15 aa, synthetic construct
RAYNTSTXLALCYAN

SEQ ID NO: 34 - protein, 15 aa, synthetic construct
  MOD_RES 8: note = Aib
NAYNTSTXLALCYAN

SEQ ID NO: 35 - protein, 15 aa, synthetic construct
  MOD_RES 8: note = Aib
RVYNTSTXLALCYAN

SEQ ID NO: 36 - protein, 15 aa, synthetic construct
  MOD_RES 8: note = Aib
NVYNTSTXLALCYAN

SEQ ID NO: 37 - protein, 15 aa, synthetic construct
  MOD_RES 8: note = Aib
RAYNTSCXLALCYAN

SEQ ID NO: 38 - protein, 15 aa, synthetic construct
  MOD_RES 8: note = Aib
NAYNTSCXLALCYAN

SEQ ID NO: 39 - protein, 15 aa, synthetic construct
  MOD_RES 8: note = Aib
RVYNTSCXLALCYAN

SEQ ID NO: 40 - protein, 15 aa, synthetic construct
  MOD_RES 8: note = Aib
NVYNTSCXLALCYAN

SEQ ID NO: 41 - protein, 15 aa, synthetic construct
  MOD_RES 8: note = Aib
RAYNTSTXLALCYAS

SEQ ID NO: 42 - protein, 15 aa, synthetic construct
  MOD_RES 8: note = Aib
NAYNTSTXLALCYAS

SEQ ID NO: 43 - protein, 15 aa, synthetic construct
  MOD_RES 8: note = Aib
RVYNTSTXLALCYAS

SEQ ID NO: 44 - protein, 15 aa, synthetic construct
  MOD_RES 8: note = Aib
NVYNTSTXLALCYAS

SEQ ID NO: 45 - protein, 15 aa, synthetic construct
  MOD_RES 8: note = Aib
RAYNTSCXLALCYAS

SEQ ID NO: 46 - protein, 15 aa, synthetic construct
  MOD_RES 8: note = Aib
NAYNTSCXLALCYAS

SEQ ID NO: 47 - protein, 15 aa, synthetic construct
  MOD_RES 8: note = Aib
RVYNTSCXLALCYAS

SEQ ID NO: 48 - protein, 15 aa, synthetic construct
  MOD_RES 8: note = Aib
NVYNTSCXLALCYAS

SEQ ID NO: 49 - protein, 18 aa, synthetic construct
  MOD_RES 10..11: note = Aib
RAYNTSTSTXXLALCYAN

SEQ ID NO: 50 - protein, 18 aa, synthetic construct
  MOD_RES 10..11: note = Aib
NAYNTSTSTXXLALCYAN

SEQ ID NO: 51 - protein, 18 aa, synthetic construct
  MOD_RES 10..11: note = Aib
RVYNTSTSTXXLALCYAN

SEQ ID NO: 52 - protein, 18 aa, synthetic construct
  MOD_RES 10..11: note = Aib
NVYNTSTSTXXLALCYAN

SEQ ID NO: 53 - protein, 16 aa, synthetic construct
  MOD_RES 8..9: note = Aib
RAYNTSCXXLALCYAN

SEQ ID NO: 54 - protein, 16 aa, synthetic construct
  MOD_RES 8..9: note = Aib
NAYNTSCXXLALCYAN

SEQ ID NO: 55 - protein, 16 aa, synthetic construct
  MOD_RES 8..9: note = Aib
RVYNTSCXXLALCYAN

SEQ ID NO: 56 - protein, 16 aa, synthetic construct
  MOD_RES 8..9: note = Aib
NVYNTSCXXLALCYAN

SEQ ID NO: 57 - protein, 18 aa, synthetic construct
  MOD_RES 10..11: note = Aib
RAYNTSTSTXXLALCYAS

SEQ ID NO: 58 - protein, 18 aa, synthetic construct
  MOD_RES 10..11: note = Aib
NAYNTSTSTXXLALCYAS

SEQ ID NO: 59 - protein, 18 aa, synthetic construct
  MOD_RES 10..11: note = Aib
RVYNTSTSTXXLALCYAS

SEQ ID NO: 60 - protein, 18 aa, synthetic construct
  MOD_RES 10..11: note = Aib
NVYNTSTSTXXLALCYAS

SEQ ID NO: 61 - protein, 16 aa, synthetic construct
  MOD_RES 8..9: note = Aib
RAYNTSCXXLALCYAS

SEQ ID NO: 62 - protein, 16 aa, synthetic construct
  MOD_RES 8..9: note = Aib
NAYNTSCXXLALCYAS

SEQ ID NO: 63 - protein, 16 aa, synthetic construct
  MOD_RES 8..9: note = Aib
RVYNTSCXXLALCYAS

SEQ ID NO: 64 - protein, 16 aa, synthetic construct
  MOD_RES 8..9: note = Aib
NVYNTSCXXLALCYAS

SEQ ID NO: 65 - protein, 16 aa, synthetic construct
RAYNTSTGGLALEYAS

SEQ ID NO: 66 - protein, 16 aa, synthetic construct
RAYNTSTGGEELEYAS

SEQ ID NO: 67 - protein, 17 aa, synthetic construct
RAYNTSTGSGEELEYAS

SEQ ID NO: 68 - protein, 17 aa, synthetic construct
  MOD_RES 9: note = E-azido-Nle
RAYNTSTGXGLALEYAS

SEQ ID NO: 69 - protein, 17 aa, synthetic construct
  MOD_RES 9: note = E-azido-Nle
RAYNTSTGXGEELEYAS

SEQ ID NO: 70 - protein, 18 aa, synthetic construct
  MOD_RES 10: note = E-azido-Nle
RAYNTSTGSXGEELEYAS

SEQ ID NO: 71 - protein, 16 aa, synthetic construct
RAYNESTGGEELEYAS

SEQ ID NO: 72 - protein, 17 aa, synthetic construct
RAYNESTGSGEELEYAS

SEQ ID NO: 73 - protein, 19 aa, synthetic construct
RAYNESTGSGSGEELEYAS

SEQ ID NO: 74 - protein, 17 aa, synthetic construct
  MOD_RES 9: note = E-azido-Nle
RAYNESTGXGEELEYAS

SEQ ID NO: 75 - protein, 18 aa, synthetic construct
  MOD_RES 10: note = E-azido-Nle
RAYNESTGSXGEELEYAS

SEQ ID NO: 76 - protein, 20 aa, synthetic construct
  MOD_RES 10: note = E-azido-Nle
RAYNESTGSXGSGEELEYAS

SEQ ID NO: 77 - protein, 16 aa, synthetic construct
RAYNRSTGGRRLRYAS

SEQ ID NO: 78 - protein, 17 aa, synthetic construct
RAYNRSTGSGRRLRYAS

SEQ ID NO: 79 - protein, 19 aa, synthetic construct
RAYNRSTGSGSGESLRYAS

SEQ ID NO: 80 - protein, 17 aa, synthetic construct
  MOD_RES 9: note = E-azido-Nle
RAYNRSTGXGRRLRYAS

SEQ ID NO: 81 - protein, 18 aa, synthetic construct
  MOD_RES 10: note = E-azido-Nle
RAYNRSTGSXGRRLCRAS

SEQ ID NO: 82 - protein, 20 aa, synthetic construct
  MOD_RES 10: note = E-azido-Nle
RAYNRSTGSXGSGESLRYAS

SEQ ID NO: 83 - protein, 20 aa, synthetic construct
  MOD_RES 10: note = E-azido-Nle
RAYNRSTGSXGSGRRLRYAS

SEQ ID NO: 84 - protein, 20 aa, synthetic construct
  MOD_RES 10: note = E-azido-Nle
RAYNESTGSXGSGESLEYAS

SEQ ID NO: 85 - protein, 20 aa, synthetic construct
  MOD_RES 10: note = E-azido-Nle
RAYNRSTGSXGSGRSLRYAS

SEQ ID NO: 86 - protein, 141 aa, synthetic construct
MIASQFLSALTLVLLIKESGAWSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTA

SEQ ID NO: 87 - protein, 14 aa, synthetic construct
WSYNTSTLALCYTA

(19) United States
(12) Patent Application Publication
(10) Pub. No.: US 2026/0074019 A1
(43) Pub. Date: Mar. 12, 2026

(54) APPROACHES TO DISCOVERING, ANALYZING, AND SYNTHESIZING COMPOUNDS THROUGH AUTOMATED IN SILICO EXPERIMENTATION

(71) Applicant: LIGANDAL, INC., Brooklyn, NY (US)

(72) Inventors: Andre WATSON, Brooklyn, NY (US); Adam STEIN, Brooklyn, NY (US); Nash RAIGLE, Brooklyn, NY (US)

(21) Appl. No.: 19/080,717

(22) Filed: Mar. 14, 2025

Related U.S. Application Data
(63) Continuation of application No. PCT/US2023/074406, filed on Sep. 15, 2023.
(60) Provisional application No. 63/375,846, filed on Sep. 15, 2022.

(51) Int. Cl. G16B 40/00; C07K 14/705; G16B 15/30

(52) U.S. Cl. CPC G16B 40/00; C07K 14/70596; G16B 15/30

(57) ABSTRACT

Introduced here is an approach to developing molecules and molecule groups via a simulated mutagenesis process that is performed as part of in silico experimentation. Due to its initiation of the simulation based on a known binding interface or predicted binding interface between two structures—whether biological or synthetic—with known sequences, the approach introduced here can accomplish linear iteration of sequences. This can be accomplished whether these sequences relate to proteins, biological amino acids, synthetic amino acids, biological nucleic acids, synthetic nucleic acids, unnatural variants thereof, or any other molecules with a three-dimensional (“3D”) structure to evaluate the thermodynamic binding and affinity of the interaction as individual nucleic acids, amino acids or individual units of a polymer are mutated one at a time.

### PRIORITY CLAIM

This application is a continuation of international application No. PCT/US2023/074406, filed Sep. 15, 2023, which claims priority to U.S. Provisional Application No. 63/375,846, filed on Sep. 15, 2022, the disclosures of which are hereby incorporated by reference herein in their entirety.

### SEQUENCE LISTING

This application contains an ST.26 compliant Sequence Listing, which is submitted concurrently in xml format via EFS-Web or Patent Center and is hereby incorporated by reference in its entirety. The .xml copy, created on Sep. 15, 2023, is named 134554-8012WO00.xml and is 92,831 bytes in size.

### TECHNICAL FIELD

Various embodiments concern computer programs and associated computer-implemented techniques for discovering compounds with therapeutic applications.

### BACKGROUND

The term “surfaceomics” may be used to refer to the study of compounds that express, present, or otherwise engage with the surfaces of cells and serve as the differentiated set of markers on a biological candidate (or simply “candidate”). Surfaceomics bridges the field of interactomics—a discipline that concerns the study of interactions between and among molecules of the cell and the consequences of those interactions. For example, a transmembrane receptor (or simply “receptor”) that is expressed 100-fold more in a given candidate relative to the next highest expressing candidate is considered a surfaceomic finding for subsequently evaluating targeting approaches for the given candidate.

The field of interactomics as relates to surfaceomics allows individuals—from healthcare professionals to researchers and developers (e.g., of therapeutics)—to predict naturally occurring interactions and non-naturally occurring interactions of various molecules with a receptor or set of receptors that are desired to be targeted for an intended application. An intended application could be, for example, a therapeutic application, diagnostic application, theragnostic application, affinity purification application, or concentration reduction application. Approaches to studying surfaceomics may not only be used to study molecules (e.g., proteins) and compounds (e.g., peptides), but also may be used to assess nuclear-, perinuclear-, mitochondrial-, and membrane-bound protein, glycoprotein, and other molecule concentrations and expression coefficients (e.g., in transcripts per million relative to an off-target organ, tissue, cell, subcellular component, extracellular compartment, or set thereof).

Generally, surfaceomics are determined via analysis of information derived through the use of single-cell ribonucleic acid (“RNA”) sequencing (“scRNA-seq”), single-nucleus RNA sequencing (“snRNA-Seq”), mass spectrometry, or enzyme-linked immunosorbent assays (“ELISAs”). However, surfaceomics could also be determined via analysis of information derived through the use of another direct affinity-interrogating approach such as those based on biolayer interferometry, surface plasmon resonance, or piezoelectric modulation of molecular-scale extrusions. Accordingly, there are various approaches to determining surfaceomics, and these various approaches have the same underlying goal, namely, establishing a better understanding of interactions along and near the surfaces of cells. However, surfaceomics—especially at the intersection of interactomics—have been developed at a relatively slow pace. While the surfaceome is more or less complete for healthy tissue in humans, the interactome only represents about five percent of this surfaceome. Deriving therapeutic insights based on surfaceomics has developed at an even slower pace.

### BRIEF DESCRIPTION OF THE DRAWINGS

FIG. 1 illustrates a network environment that includes a development platform that is executed by a computing device.

FIG. 2 illustrates an example of a computing device that is able to implement a development platform designed to develop therapeutically relevant molecules.

FIG. 3 includes a high-level illustration of a workflow that can be implemented by the development platform.

FIG. 4 includes a high-level illustration of a workflow that can be implemented by the specificity module.

FIG. 5 includes an example of an interface through which a user can interact with a specificity module.

FIG. 6 includes an example of an interface through which the user is able to select the cell type, tissue, or organ of interest.

FIG. 7 includes an example of an interface that shows a typical output for an organ search conducted by the specificity module.

FIG. 8 shows how the user may be able to save the search results produced by the specificity module.

FIG. 9 includes an example of an interface that shows how the user can refine the search results to more specific interactome patterns.

FIG. 10 shows how the refined search results could be initially filtered and clustered to the top n sequences, where n equals 280.

FIG. 11 shows how the top n sequences can then be filtered and clustered to the top m sequences, where m equals 50.

FIG. 12 shows how the top candidate (i.e., the UMOD gene) can be selected from among the remaining top m sequences.

FIG. 13 includes a high-level illustration of a workflow that can be implemented by a docking module.

FIG. 14 includes an example of an interface through which a user can interact with the docking module.

FIG. 15 includes explanations of parameters for controlling the operations that are performed by the docking module.

FIG. 16 shows an example of a tensor matrix that may be used by the docking module.

FIG. 17 shows how the docking module may use the tensor matrix in operation.

FIG. 18 shows an example of an output that may be produced by a weighting system.

FIG. 19 illustrates how a user may be able to access the visual tool, and the starting data that is subsequently processed for interactions with the option of being fed into one or more neural network or deep learning approaches.

FIG. 20 illustrates how the docking module could process binding patches between the top candidate (here, a first protein) and a target (here, a second protein) in order to identify an optimal docking interface.

FIG. 21 shows an example of an interaction energy calculator that can be used by the docking module.

FIG. 22 shows an example of a nearest neighbor calculator that can be used by the docking module.

FIG. 23 includes example code for training the machine learning model in accordance with some embodiments.

FIG. 24 includes example code for reinforcement of the machine learning model.

FIG. 25 shows an example of an initial output that can be generated by the docking module.

FIG. 26 shows the results of an “run” by the docking module.

FIG. 27 shows an example of a structural model produced for a protein.

FIG. 28 shows an example of another structural model produced for the protein but with hydrogens appended thereto.

FIG. 29 shows an example of a structural model for the crystal structure of the protein.

FIG. 30 illustrates how the docking module can overlay its generated structural model on the known structure of the protein.

FIG. 31 shows an example of an interface through which a user is able to select a peptide or collection of peptides for which mutagenesis is to be performed.

FIGS. 32A-B include examples of visualizations that may be produced (e.g., by a visualization module) based on outputs produced by the mutagenesis algorithm or analyses of the outputs by a design module.

FIGS. 33A-B include visualizations of mutated structure files, as generated by a mutagenesis module, with mutated residues determined by the mutagenesis algorithm.

FIGS. 34A-B show only the mutated residues determined by the mutagenesis algorithm.

FIGS. 35A-B include examples of data structures (here, spreadsheets) in which the outcome of the mutagenesis is documented.

FIG. 36 includes a high-level illustration of a process by which a compound can be developed.

FIG. 37 includes a block diagram of a processing system in which at least some operations described herein can be implemented.

Various embodiments are shown in the drawings for the purpose of illustration. However, those skilled in the art will recognize that alternative embodiments may be employed without departing from the principles of the present disclosure. Accordingly, while certain embodiments are shown in the drawings, the technologies described herein are amenable to various modifications.

### DETAILED DESCRIPTION

A key issue in the study of surfaceomics is that experimental data—for example, in the form of x-ray crystallography data or cryo-electron microscopy data—for native interactions between a target molecule (e.g., a protein) and other molecules or compounds is available for only about five percent of the human proteome. This applies to the entire human and non-human proteome, not only to the receptor surfaceome. Using machine learning, about 2,886 receptors have been identified within the human proteome but only a small portion of these receptors—perhaps several hundred—have had plausible native interactions demonstrated.

By integrating interactomics data obtained through empirical studies, even in the absence of precise docking conformations, it is possible to predict the likely docking sites of cell surface markers (or simply “markers”) with native biological molecules, non-native biological molecules, and non-native synthetic molecules. Historically, the mere knowledge of a receptor or a marker corresponding to a target surfaceome profile was insufficient to design groups of molecules (also called “molecule groups,” “molecule sets,” or “molecule strings”). In fact, development was largely limited to designing molecule groups using antibody-based approaches or small molecule screening approaches. However, antibodies or other molecules discovered via these approaches may not necessarily bind to the appropriate surface of a receptor, as determined in comparison to the native behavior of the receptor and any of its orthosteric sites or allosteric sites, where relevant. Accordingly, not only are antibodies or other molecules discovered slowly via these approaches because “brute force” (e.g., trial and error) is heavily relied upon, but these antibodies or other molecules still may not bind to the appropriate surface of the receptor as intended, leading to further costs and delay.

Another approach that has traditionally been employed in an effort to design molecule groups is random sequence generation. At a high level, random sequence generation involves using a computer program—commonly called a “random sequence generator”—to generate random deoxyribonucleic acid (“DNA”), RNA, or protein sequences in an effort to rapidly develop molecule groups. Another commonly used set of approaches is phage display, yeast display, DNA-barcoded peptide display, and the like, whereby a comprehensive set of sequences is synthesized, and the molecular variants that bind to the intended target are isolated and sequenced. Typically, these approaches are limited to iterating over a very short peptide sequence, for example, 10-mers that result in 1.024×10¹³ (20¹⁰) sequences for the 20 canonical amino acids and a 10-mer peptide sequence. While design and discovery of molecule groups is much quicker with random sequence generation and relatively quick with display and barcoding approaches, these approaches suffer from the same downside, namely, these molecule groups may not bind to the appropriate surface of the receptor as intended and, in the case of polymeric screens (including polypeptides), are limited in their secondary and tertiary structural features due to short sequence lengths preventing more complex folding or binding characteristics to the desired target interface. Moreover, with random sequence generation, predicting whether molecule groups have a therapeutic effect tends to be more difficult since there may be few, if any, rules governing how the molecule groups are generated and the computational complexity of generating peptide, DNA, RNA, or glycoprotein folding increases hyper-exponentially with sequence length.

Neither of these approaches address the structural challenges of rapidly designing a molecule or molecule group that is to engage a surface of a given candidate.

Introduced here, therefore, is an approach to developing molecules and molecule groups via a simulated mutagenesis process that is performed as part of in silico experimentation. Conventional mutagenesis requires exponentially more time and resources as the underlying nucleic acid, polymer, or protein sequence (or simply “sequence”) elongates, largely due to the need to predict structure and binding of an exponentially growing state space. For example, 20ⁿ possible sequences can lead to computational overload for simulating amino acids as a protein of length n becomes longer. The approach introduced here, due to its initiation of the simulation based on a known binding interface or predicted binding interface between two structures—whether biological or synthetic—with known sequences, can accomplish linear iteration (i.e., O(n)) of sequences. This can be accomplished whether these sequences relate to proteins, biological amino acids, synthetic amino acids, biological nucleic acids, synthetic nucleic acids, unnatural variants thereof, or any other molecules with a three-dimensional (“3D”) structure to evaluate the thermodynamic binding and affinity of the interaction as individual nucleic acids, amino acids or individual units of a polymer are mutated one at a time. The optimized sequence can then be inferred through the substitutions of individual nucleic acids, amino acids, or other individual units of a polymer. Such an approach allows for the development of molecules and molecule groups (e.g., peptides) while greatly reducing the computation time and resource requirements of optimization. In comparison to random sequence generation or display approaches, such an approach also lessens the overall “cost” of computational resources or experimental complexity needed to discover molecules and molecule groups as interactivity (and other aspects as discussed below) can be considered during the development process rather than used as a means to filter randomly generated or displayed sequences.

This approach can be implemented by a computer program that implements a series of modules that, in combination, allow a user to be guided through the process by which molecules or molecule groups are automatically discovered, analyzed, and designed through mutagenesis—the process by which the DNA, protein, or polymer's individual units change, resulting in sequence mutation. Rather than mutate the sequence at the DNA level, the computer program could instead change the sequence at the protein or individual mer level in some embodiments. As such, the computer program may be referred to as a “precision medicine development platform” or simply “development platform,” which utilizes predictive interactomics for discovering, designing and developing an interacting molecule with a given protein target in some embodiments. Users of the development platform can include healthcare professionals interested in better understanding whether a given molecule or molecule group is likely to have a therapeutic effect for a patient or patient cohort, as well as researchers interested in better understanding the surfaceomics and/or interactomics of a given molecule or molecule group and developers interested in better understanding whether there is commercial potential for a given molecule or molecule group.

As further discussed below, the development platform can be integrated with one or more databases with data (e.g., regarding candidates, diseases, etc.) stored therein. With this data, the development platform may be able to flexibly evaluate mutations; affected cells, tissues, and organs; and surfaceome of the candidate that corresponds to the disease state of one or more diseases. Such an implementation allows for rapid evaluation of potential candidates for clinical translation, whereby a company may choose a set of diseases only affecting one cell, tissue, or organ or affecting a set of cells, tissues, or organs. For example, these database(s) may include data relating to tens of thousands, hundreds of thousands, or millions of mutations corresponding to the affected cells, tissues, or organs. This approach may also be applied to surfaceomics data that does not relate to a cell, tissue, or organ; such as the surfaceome of a virus, bacteria, eukaryote, or prokaryote.

In the case of genetic diseases, combined deployment of the development platform and database(s) allows for rapid tailoring of gene therapy, gene editing, and gene modulating approaches—as well as a small molecule, macromolecular, or biologic delivery approaches—that can achieve a higher therapeutic effect, for example, through enhanced biodistribution; cell, tissue, or organ trophism; safety; and efficacy. The ability to characterize and empirically assess binding to target markers and the surfaceome of a diseased cell, tissue, or organ (or even a healthy cell, tissue, or organ requiring some form of reprogramming, for example, for implementing immunotherapies; killing cancer cells, senescent cells, or other cells; or modulating functions corresponding to targeting of an antigen for autoimmune purposes, allergy purposes, etc.) allows for a flexible approach to developing molecules and molecule groups. These molecules may be representative of carbohydrates, lipids, proteins, polymers, polymer-ligand conjugates, or nucleic acids, and these molecule groups may be representative of, or part of, small molecules, peptides, peptoids, polymers, polymer-drug conjugates, other ligands, and the like. Accordingly, these molecules and molecule groups could be used not only in development of biopharmaceuticals (also called “biologics”), but also in synthetic compound development as it relates to antibody-drug conjugates, peptide-drug conjugates, nanoparticle delivery systems, polymer-drug conjugates, polymer-peptide conjugates, lipid-polymer-peptide conjugates, recombinant protein conjugates, and other multimeric molecular or multi-block polymer conjugates.

For the purpose of illustration, embodiments may be described in the context of developing peptides for therapeutic applications. However, those skilled in the art will recognize that the features of these embodiments may be similarly applicable to the development of other compounds that comprise amino acids, like small molecules, peptoids, ligands, and the like.

Embodiments may also be described in the context of executable instructions for the purpose of illustration. However, those skilled in the art will recognize that aspects of the technology could be implemented via hardware or firmware instead of, or in addition to, software.

### Terminology

References in the present disclosure to “an embodiment” or “some embodiments” means that the feature, function, structure, or characteristic being described is included in at least one embodiment. Occurrences of such phrases do not necessarily refer to the same embodiment, nor do they necessarily refer to alternative embodiments that are mutually exclusive of one another.

The term “based on” is to be construed in an inclusive sense rather than an exclusive sense. That is, in the sense of “including but not limited to.” Thus, the term “based on” is intended to mean “based at least in part on” unless otherwise noted.

The terms “connected,” “coupled,” and variants thereof are intended to include any connection or coupling between two or more elements, either direct or indirect. The connection or coupling can be physical, logical, or a combination thereof. For example, elements may be electrically or communicatively connected to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing multiple tasks.

The term “about” means within ±10% of the recited value.

The term “transmembrane receptor” may refer to any receptors that are embedded in the plasma membrane of cells. Transmembrane receptors act in cell signaling or mediate intracellular interactions by receiving—and binding to—extracellular molecules, such as hormones, neurotransmitters, cytokines, growth factors, cell adhesion molecules, other transmembrane receptors, antigens, soluble proteins, or nutrients. Transmembrane receptors are integral membrane proteins that allow communication between the cell and the extracellular space. Note that the term “transmembrane receptor” could be used interchangeably with “cell surface receptor,” “membrane receptor,” or simply “receptor.”

The term “biological candidate” may refer to a nucleic acid, gene, carbohydrate, glycoprotein, glycosaminoglycan, lipid, protein, binary or ternary complex thereof, cell, tissue, organ, or a set of nucleic acids, genes, proteins, carbohydrates, glycoproteins, glycosaminoglycans, lipids, binary or ternary complex thereof (comprising 2, 3, or more biological candidates simultaneously interacting), cells, tissues, or organs that are of interest. Commonly, the biological candidate will be the target of a molecule or molecule group. Said another way, the biological candidate may be whatsoever within a living body, eukaryotic cell, prokaryotic cell, or virus to which the molecule or molecule group binds, resulting in a modification in function or behavior. Note that the term “biological candidate” could be used interchangeably with “biological target” or simply “candidate” or “target.”

The terms “cell surface markers” or simply “markers” may refer to proteins that are expressed on the cellular surface or carbohydrates, glycoproteins, glycosaminoglycans, lipids, or other biological substrates that attach to the cellular membrane. Markers are commonly used for classification (e.g., as part of a flow cytometry operation).

The term “surfaceome” may refer to the entire complement of molecules that can be found along the surface of a given candidate.

The term “proteome” may refer to the entire complement of proteins that is, or can be, expressed by a given candidate or given organism. For example, the “human proteome” can include all expressed proteins in a human, at a given time, under defined conditions.

The term “interactome” may refer to the entire set of molecular interactions in a cell. The term is generally used to refer to physical interactions among molecules (e.g., proteins) but can also be used to describe sets of indirect interactions (e.g., among genes). Interactomics as relate to surfaceomics is the study of interactions between a surfaceome and other biological molecules, whether they are protein, DNA, RNA, small molecules, sugars, lipids, or other biological matter.

### Overview of Development Platform

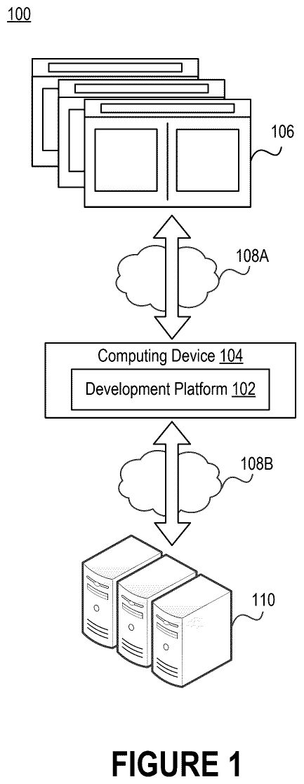

FIG. 1 illustrates a network environment 100 that includes a development platform 102 that is executed by a computing device 104. An individual (also referred to as a “user”) can interact with the development platform 102 via interfaces 106. The user could be a healthcare professional, researcher, or developer (e.g., (e.g., of therapeutics), for example. Depending on the nature and interests of the user accessing the interfaces 106, the interfaces 106 may allow for the review of data, examination of outputs produced by the development platform 102, initiation of molecular synthesis based on the outputs produced by the development platform 102, and management of preferences. Some interfaces may serve as informative dashboards through which individual users can observe, manage, or guide the process by which the development platform 102 develops molecules and molecule groups, while other interfaces may facilitate interactions between multiple users (e.g., who are members of the same research team or development team).

As shown in FIG. 1, the development platform 102 can reside in a network environment 100. Thus, the computing device 104 on which the development platform 102 resides can be connected to one or more networks 108A-B. Depending on its nature, the computing device 104 could be connected to a personal area network (“PAN”), local area network (“LAN”), wide area network (“WAN”), metropolitan area network (“MAN”), or cellular network. For example, if the computing device 104 is a computer server, then the computing device 104 may be accessible to users via respective computing devices (e.g., mobile phones or laptop computers) that are connected to the Internet via LANs. The data to be examined by the development platform 102 may be obtained from the respective computing devices or obtained from elsewhere (e.g., one or more databases that are accessible to the computing device).

Additionally or alternatively, the computing device 104 may be connected to one or more other computing devices over a short-range wireless connectivity technology, such as Bluetooth®, Near Field Communication (“NFC”), Wi-Fi® Direct (also referred to as “Wi-Fi P2P”), and the like. As an example, the development platform 102 could be embodied as a desktop application that is executed by a laptop computer. In such embodiments, the laptop computer may be communicatively connected—via a wireless communication channel—to a source from which to acquire data. The source could be a database that is accessible to the laptop computer via a network (e.g., the Internet). The data could alternatively be obtained from another computer program executing on the laptop computer and optionally connected to one or more instruments that are able to generate quantitative or qualitative experimental data or synthesize and/or purify a given molecular candidate or set of candidates.

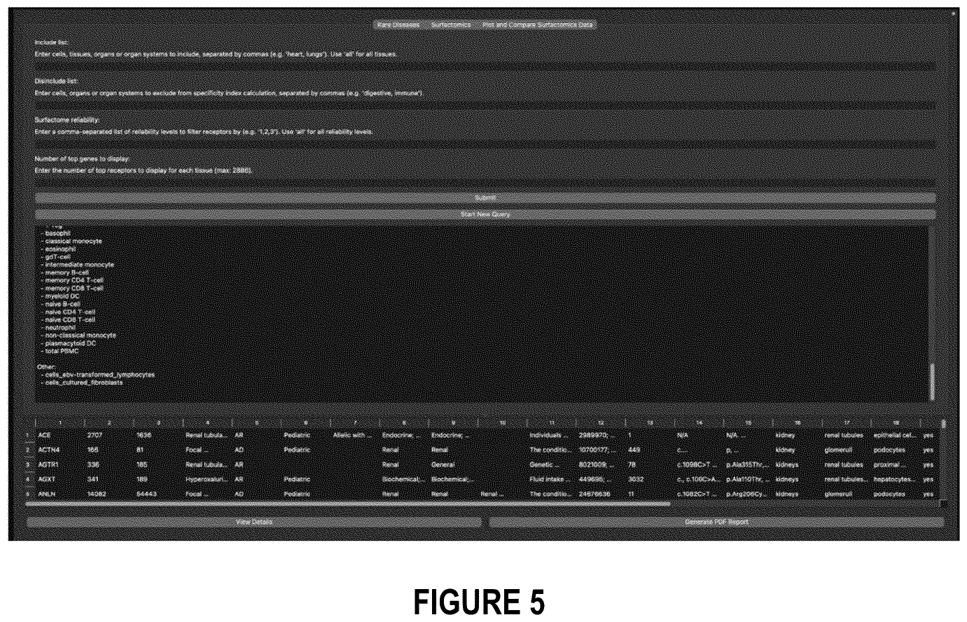

The interfaces 106 may be accessible via a web browser, desktop application, mobile application, or another form of computer program. For example, a user may be able to access interfaces through which to guide development of a molecule or molecule group via a desktop application executing on a laptop computer as mentioned above. As another example, a user may be able to access interfaces through which information regarding molecules or molecule groups can be reviewed via a web browser. Several examples of interfaces 106 generated by the development platform 102 and outputs to be presented thereon are further discussed below with reference to FIGS. 5-12 and 14-35B. Accordingly, the interfaces 106 generated by the development platform 102 may be accessible on various computing devices, including mobile phones, tablet computers, desktop computers, and the like.

Generally, the development platform 102 is executed—at least partially—by a cloud computing service operated by, for example, Amazon Web Services®, Google Cloud Platform™, or Microsoft Azure®. Thus, the computing device 104 may be representative of a computer server that is part of a server system 110. Often, the server system 110 is comprised of multiple computer servers. These computer servers can include different types of data (e.g., proteomic data, surfaceomic data, interactomic data, protein data, cell type data, tissue data, organ data), algorithms for processing incoming data, machine learning models for discovering molecules and molecule groups, and other assets. Those skilled in the art will recognize that these data could also be distributed among the server system 110 and one or more computing devices. As an example, data that is obtained (e.g., acquired or generated) by a user may be stored on, and processed by, her own computing device for security or privacy purposes. This may be useful if, for example, the user is a healthcare professional who is interested in reviewing whether a molecule or molecule group developed by the development platform 102 is suitable for a patient based on analysis of her physiological data, clinical data, DNA sequencing data, scRNA-seq data, proteomics data, etc. As another example, this may be useful if the user is a developer who is interested in utilizing sensitive information (e.g., subject to trade secret protections) in establishing whether a molecule or molecule group is a suitable candidate for a therapeutic application.

In some embodiments, the development platform 102 is executed—at least partially—by a computing device that exploits quantum mechanical phenomena. This quantum computing device (also called a “quantum computer”) may have multiple superconducting qubits. Each qubit may represent a two-state system, like the classical bits employed by conventional computing devices, except that it can exist in a superposition of its two states. For example, the development platform 102 may reside on a quantum computer, such that processing of data occurs in a quantum environment. Additionally or alternatively, the databases from which the data is obtained may reside on one or more quantum computers, such that the data is stored and/or accessed in a quantum environment.

Components of the development platform 102 could also be hosted locally. That is, part of the development platform 102 may reside on the computing device used to access one of the interfaces 106. For example, the development platform 102 may be embodied as a desktop application executing on a laptop computer as mentioned above. Note, however, that the desktop application may be communicatively connected to the server system 110 on which other components of the development platform 102 are hosted.

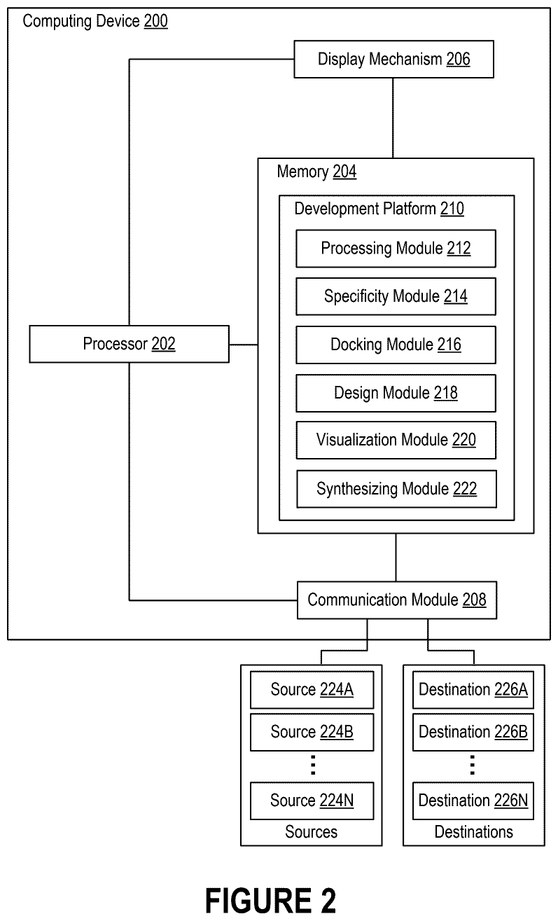

FIG. 2 illustrates an example of a computing device 200 that is able to implement a development platform 210 designed to develop therapeutically relevant molecules. As shown in FIG. 2, the computing device 200 can include a processor 202, memory 204, display mechanism 206, and communication module 208. Each of these components is discussed in greater detail below.

Those skilled in the art will recognize that different combinations of these components may be present depending on the nature of the computing device 200. For example, if the computing device 200 is a computer server that is part of a server system (e.g., server system 110 of FIG. 1), then the computing device 200 may not include the display mechanism 206. Conversely, if the computing device 200 is a laptop computer, then the computing device 200 may include the display mechanism 206.

The processor 202 can have generic characteristics similar to general-purpose processors, or the processor 202 may be an application-specific integrated circuit (“ASIC”) that provides control functions to the computing device 200. In embodiments where the computing device 200 is a quantum computer, the processor 202 could be a quantum processing unit (“QPU”) that is based on a quantum circuit and quantum logic gate-based model of computing. As shown in FIG. 2, the processor 202 can be coupled to all components of the computing device 200, either directly or indirectly, for communication purposes.

The memory 204 can be comprised of any suitable type of storage medium, such as static random-access memory (“SRAM”), dynamic random-access memory (“DRAM”), electrically erasable programmable read-only memory (“EEPROM”), quantum memory, flash memory, or registers. In addition to storing instructions that can be executed by the processor 202, the memory 204 can also store data generated by the processor 202 (e.g., when executing the modules of the development platform 210). Note that the memory 204 is merely an abstract representation of a storage environment. The memory 204 could be comprised of actual integrated circuits (also called “chips”).

The display mechanism 206 can be any mechanism that is operable to visually convey information to a user. For example, the display mechanism 206 can be a traditional panel that includes light-emitting diodes (“LEDs”), organic LEDs, liquid crystal elements, or electrophoretic elements. As another example, the display mechanism 206 could be part of an augmented reality system or virtual reality system. As further discussed below, outputs produced by the development platform 210 (e.g., through execution of its modules) can be posted to the display mechanism 206 for review by a user of the computing device 200.

The communication module 208 may be responsible for managing communications external to the computing device 200. Here, for example, the communication module 208 is able to establish separate communication channels with sources 224A-N from which to obtain data that can be processed, analyzed, or otherwise used by the development platform 210. This data may include information regarding proteins, cells, tissues, organs, surfaceomics of those structures, structural data, proteomic data, and the like. Note that for each source, a separate script—stored in the memory 204 as part of the development platform 210—may be executed by the processor 202 to allow the communication module 208 to retrieve data therefrom. The sources 224A-N may be representative of databases from which data can be acquired, by the development platform 210, via the communication module 208.

The communication module 208 can be wireless communication circuitry that is able to establish wireless communication channels with other computing devices. Examples of wireless communication circuitry include 2.4 gigahertz (“GHz”) and 5 GHz chipsets compatible with Institute of Electrical and Electronics Engineers (“IEEE”) 802.11—also referred to as “Wi-Fi chipsets.” Alternatively, the communication module 208 may be representative of a chipset configured for Bluetooth, NFC, and the like. Some computing devices—like mobile phones, tablet computers, and the like—are able to wirelessly communicate via separate channels, while other computing devices—like computer servers—tend to wirelessly communicate via a single channel. Accordingly, the communication module 208 may be one of multiple communication modules implemented in the computing device 200, or the communication module 208 may be the only communication module implemented in the computing device 200.

The nature, number, and type of communication channels established by the computing device 200—and more specifically, the communication module 208—can depend on (i) the sources 224A-N from which data is acquired by the development platform 210 and (ii) the destinations 226A-N to which data is transmitted by the development platform 210. Assume, for example, that the development platform 210 resides on a computer server. In such embodiments, the communication module 208 can communicate with one or more sources external to the computing device 200 from which to obtain data. These sources could be network-accessible databases, for example. Moreover, the communication module 208 may communicate with at least one destination to which analyses of the data—or the data itself—are transmitted. As an example, this destination could be instrumentation (e.g., a peptide synthesizer) that is able to synthesize a molecule or molecule group developed by the development platform 210. As another example, this destination could be another computing device that is associated with a user interested in the analyses produced by the development platform 210. Those skilled in the art will recognize that a given computing device—say, a laptop computer or a computer server—could be a source and destination.

With the communication module 208, the development platform 210 can integrate not only with structured data that are stored locally in the memory 204 but also structured data that are external to the computing device 200. For example, the development platform may seamlessly integrate with comprehensive databases containing data related to known drugs, disease-causing mutations, surfaceomic datasets, interactomics datasets, other multi-omics datasets, therapeutic targets, market information, and the like. This integration aids in the identification of relevant candidates and facilitates comparisons with existing treatments. As further discussed below, advanced capabilities relating to analysis of structural data, surfaceomic data, proteomic data, and interactomic data enhance the ability of the development platform 210 to deliver targeted solutions to problems that have not traditionally been solvable (and, in some cases, may not even have been known).

For convenience, the development platform 210 is referred to as a computer program that resides within the memory 204. However, the development platform 210 could be comprised of hardware or firmware instead of, or in addition to, software. In accordance with embodiments described herein, the development platform 210 can include a processing module 212, specificity module 214, docking module 216, design module 218, visualization module 220, and synthesizing module 222. These modules could be integral parts of the development platform 210, or these modules could be logically separate from the development platform 210 but operate “alongside” it. Together, these modules enable the development platform 210 to develop peptides or other molecules with therapeutic effects that can be readily produced. As mentioned above, embodiments may be described in the context of developing peptides for the purpose of illustration; however, those skilled in the art will recognize that the features of those embodiments are not limited to developing peptides.

At a high level, the development platform 210 is driven by artificial intelligence and revolutionizes the process of developing peptides having therapeutic applications. The modules of the development platform 210 can collectively perform operations that encompass the initial design of the peptides, optimization of the peptides, and manufacture (e.g., synthesis) of the peptides. Thus, outputs produced by the development platform 210 could be integrated into appropriate instrumentation (e.g., a peptide synthesizer) to enable end-to-end development of therapeutic compounds on a scale—and at a rate—that is unprecedented.

The processing module 212 can process data that is obtained by the development platform 210 into a format that is suitable for the other modules. For instance, the processing module 212 can apply operations to data obtained from different sources in preparation for analysis by the other modules of the development platform 210. As an example, the processing module 212 could filter or alter surfaceomic or interactomic data from different sources or the processing module 212 could concatenate the surfaceomic or interactomic data from different sources in a single data structure, such that the surfaceomic or interactomic data can be more readily analyzed despite being obtained from more than one source. As another example, the processing module 214 may parse surfaceomic data and structural data associated with a peptide and then concatenate these data in a single data structure, such that these data can be more easily retrieved, analyzed, and stored—even if these data are obtained from more than one source. As another example, the processing module 214 may obtain patient data that is uploaded by a healthcare professional and then compared to existing structures to enable a personalized therapeutic application (e.g., to treat cancer discovered through a biopsy). Accordingly, the processing module 214 may be responsible for ensuring that the appropriate data is accessible to the other modules of the development platform 210.

The specificity module 214 may be responsible for generating sequences for candidate molecules (e.g., peptides) with precision, taking into account features such as cell-, tissue-, and organ-type specificity. Assume, for example, that the specificity module 214 receives input that is indicative of a selection of an organ, a tissue, or a cell type of interest. Upon receiving the input, the specificity module 214 may generate multiple sequences that are representative of different peptides. Specifically, the specificity module 214 may apply a first machine learning model or database-processing model to data corresponding to the selected organ, tissue, or cell type. The first machine learning model may be designed and trained to predict protein-protein interactions along and near the surfaces of the organ, tissue, or cell type of interest, and may also be a database-driven approach to parsing existing data on protein-protein interactions along and near the surfaces of the organ, tissue, or cell type of interest. For example, the first machine learning model may be trained on a training dataset that includes information regarding known protein-protein interactions determined through x-ray crystallography data or cryo-electron microscopy data. In embodiments where machine learning is not used—for example, where the specificity module 214 employs a database-processing model or some other heuristic, rule, or algorithm—existing protein-protein interactions or other intermolecular interactions could be parsed for derivative sequences or binding motifs.

In some embodiments, the first machine learning model is a deep learning model. The term “deep learning” is commonly used to refer to a broader set of machine learning algorithms and models that are based on artificial neural networks (or simply “neural networks”) with reinforcement learning, while the term “deep” refers to the use of multiple layers in the neural networks. These multiple layers may progressively extract higher-level features from the input, which in this case may be the data corresponding to the selected organ, tissue, or cell type. With the use of a deep learning model, the specificity module 214 can ensure highly targeted design of sequences for candidate peptides.

The docking module 216 may be responsible for employing a second machine learning model that predicts, for the candidate peptides for which sequences are generated by the specificity module 214, binding interfaces (also called “docking interfaces”). As mentioned above, native interactions are known for only about five percent of the human proteome, and therefore these docking interfaces are generally predicted for previously unknown interactions. Assume, for example, that the specificity module 214 outputs multiple sequences of amino acids, each of which is representative of a candidate peptide. For each sequence of amino acids (and thus, each candidate peptide), the second machine learning model can predict the docking interfaces for peptide-ligand interactions. Like the first machine learning model, the second machine learning model may be a deep learning model that is based on a neural network. However, the second machine learning model may be based on a neural network that is optimized on known interactions using reinforcement learning. For example, the desired target ligand for a candidate peptide may be randomly rotated and translated and then a reward-and-punishment reinforcement learning (“RL”) algorithm could be used to train weights of the second machine learning model for subsequent restoration of the original docking site. With the use of a deep learning model, the docking module 216 can more accurately predict docking sites, facilitating the design and identification of potential therapeutic candidates from among the peptides generated by the specificity module 214.

The design module 218 may be responsible for building on the results of the docking module 216. Assume, for example, that the specificity module 214 produces, as output, multiple sequences corresponding to different peptides while the docking module 216 produces, as output, indications of predicted docking sites for those different peptides. In such a scenario, the design module 218 can identify a sequence from among the multiple sequences based on an analysis of the predicted docking sites (and, in some embodiments, data related to docking capabilities of the different peptides). Such an approach streamlines the process of generating peptide templates, ensuring a focused approach to the development of potential therapeutic candidates. The terms “peptide template,” peptide scaffold,” and “polypeptide motif” may refer to a data structure that documents characteristics of a peptide in a more stable manner and that allows for controlled, consistent synthesis of the peptide. These characteristics can include size, shape, facet structure, amino acid composition, predicted binding behavior, and the like.

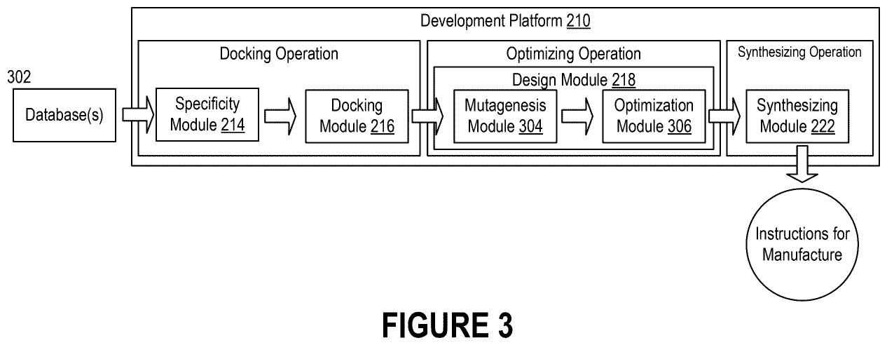

Note that, in some embodiments, the design module 218 can include, or is accessible to, a mutagenesis module 304 and/or an optimization module 306 as shown in FIG. 3.

The mutagenesis module 304 may be responsible for implementing a mutagenesis algorithm (e.g., a single-point mutagenesis algorithm) that introduces, to each candidate peptide, mutations (e.g., single-point mutations) across the interacting surface between that candidate peptide and its native interacting ligand or simulated interacting ligand. The collection of mutated peptides can then be assessed in silico for predicted binding affinity. With the collection of mutated peptides, the mutagenesis module 304 may “stitch” together the mutated peptides in such a way that the most thermodynamically favorable string of sequences is ultimately generated. Such an approach to mutagenesis can lead to O(n) compute time. For example, for n binding sites, m possible amino acids (e.g., natural and unnatural amino acids, as well as peptoid or other single-mer motifs that can be substituted in) can be iterated through. This means that for a 20-mer binding sequence, if the mutagenesis module 304 is considering 20 possible natural amino acids, 400 possible structures (i.e., 20×20 or m*n) would be considered rather than 1.049×10²⁶ (i.e., 20²⁰ or mⁿ).

The optimization module 306, meanwhile, may be responsible for enhancing properties such as solubility, binding affinity, and delivery mechanism, thereby finetuning the candidate peptides for improved therapeutic efficiency. To accomplish this, the optimization module 306 may monitor, compute, or estimate these properties as mutations are introduced by the mutagenesis module 304.

Users can benefit from the ability to visualize data that is obtained and generated by the modules of the development platform 210 to empower informed decision making throughout the development process. The visualization module 220 may be responsible for generating interfaces to which these data—and analyses of these data—can be posted for review. Several examples of interfaces are shown in FIGS. 5-12 and 14-35B, and these interfaces allow users to be guided through the development process, though in an interactive manner that allows for exploration of sequence-activity relationships, solubility profiles, relevant interfaces between two or more molecules, optimization strategies, and the like.

The synthesizing module 222 may be responsible for taking insights gleaned into candidate peptides and facilitating manufacture of those candidate peptides if desired. Assume, for example, that the design module 218 identifies a single sequence (and thus, a single polypeptide) from among the multiple sequences generated by the specificity module 214 as the preferred candidate for a therapeutic application. In such a scenario, the synthesizing module 222 may create, compile, or otherwise document instructions for the manufacture (e.g., synthesis and purification) of the peptide. Through the creation of targeted instruction sets, the synthesizing module 222 can enable the rapid, scalable production of high-quality peptides with therapeutic applications, allowing for large-scale manufacturing at a rate much quicker than has conventionally been possible. With the synthesizing module 222, the development platform 210 may be able to allow for large-scale manufacturing of peptides in less time than has conventionally been possible. For example, each mer could be synthesized in 30-90 seconds, meaning that insights can be gleaned in a matter of minutes and hours (and days, in the case of experimentation), rather than weeks or months.

FIG. 3 includes a high-level illustration of a workflow that can be implemented by the development platform 210. As shown in FIG. 3, the workflow can involve the development platform 210 executing a series of operations, namely, a docking operation, an optimizing operation, and a synthesizing operation, in order.

Initially, the development platform 210 can integrate with one or more databases 228. For example, the development platform 210 may implement, or cause execution of, a first script (e.g., written in a programming language such as Python) that initiates and maintains communication with the database(s) 228. From the database(s) 228, the development platform 210 can obtain data to be used to generate sequences for candidate molecules (e.g., peptides) and determine whether those candidate molecules will have sufficient docking activity and have anticipated therapeutic applications. The data could include cell type data, tissue data, organ data, surfactomic data, protein data, structural data, proteomic data, or any combination thereof.

Generally, data is compiled by the development platform 210 by integrating with multiple public databases, though data could be compiled by the development platform 210 by integrating with private databases instead of, or in addition to, the multiple public databases. Data may also be compiled by direct-to-patient diagnostics or through accessing such diagnostic data, whereby such data may be genomic (DNA or RNA sequencing), proteomic, glycomic, or multi-omic. As an example, the development platform 210 may integrate with the Protein Data Bank (“PDB”) database from which 3D structural data of large molecules, such as proteins and nucleic acids, can be obtained and the AlphaFold database from which 3D structural data of proteins across different proteomes can be obtained.

Then, the development platform 210 can implement the specificity module 214 and docking module 216 as part of a docking operation. As part of the docking operation, the docking module 216 may implement, or cause execution of, a second script. The second script may be written in the same programming language as the first script, the execution of which initiates and maintains communication with the database(s) 228. At a high level, execution of the second script may result in one or more machine learning models being applied against data obtained from the database(s) 228 and/or information derived from analysis of the data (e.g., by the specificity module 214 or docking module 216). For example, the specificity module 214 may apply a first machine learning model against data obtained from the database(s) 228, so as to produce multiple sequences corresponding to different peptides, and the docking module 216 may apply a second machine learning module against the multiple sequences, so as to produce indications of predicted docking activity of the different peptides.

In embodiments where one or more modules of the development platform 210 utilize machine learning, these machine learning models could be further trained or tuned using RL algorithms. Built upon the inspirations of brain neuroscience, RL algorithms are designed to learn to solve a multi-level problem—here, how to generate peptides and determine which peptides are suitable therapeutic candidates based on docking activity—by trial and error. At a high level, reinforcement learning concerns an autonomous agent taking suitable actions to maximize rewards in a particular environment. Over time, the agent learns from its experiences and tries to adopt the best possible behavior. Examples of RL algorithms include the Monte Carlo algorithm, Q-learning algorithm, State-Action-Reward-State-Action (“SARSA”) algorithm, Q-learning—Lambda algorithm, SARSA—Lambda algorithm, Deep Q Network (“DQN”) algorithm, Deep Deterministic Policy Gradient (“DDPG”) algorithm, Asynchronous Advantage Actor-Critic (“A3C”) algorithm, Q-Learning with Normalized Advantage Functions (“NAF”) algorithm, Trust Region Policy Optimization (“TRPO”) algorithm, Proximal Policy Optimization (“PPO”) algorithm, Twin Delayed Deep Deterministic Policy Gradient (“TD3”) algorithm, and Soft Actor-Critic (“SAC”) algorithm. Generally speaking, any RL algorithm that can selectively utilize data such as positional data, rotational data, 3D data, four-dimensional (“4D”) data, five-dimensional (“5D”) data, n-dimensional (“nD”) data, or other multiparametric datasets and where at least one of the inputs can be a predictive interaction (e.g., thermodynamic assessment) are preferred. Some RL algorithms may be better suited for discrete action spaces, continuous action spaces, and discrete-continuous hybrid action spaces, however. For example, finding the optimal rotation and translation of a given chain, or utilizing a dynamic process for discovering a docking site, may be more well suited for continuous action spaces (e.g., DDPG, TD3, SAC). Once a docking site is found, utilizing DQN or A3C for evaluating a discrete action space may be used. All of the methods outlined herein are useful for high-dimensional data and have various implications for evaluating data based on the completeness of the possible state-space within a given simulation space. Various RL algorithms may be used to obtain convergence and a stable algorithmic approach for the training agent reaching an acceptable state of “learning” for further use in subsequent predic

Through execution of the second script, the docking module 216 can predict protein-protein or other intramolecular interactions along and near the surfaces of the cell, tissue, or organ of interest. In some embodiments, execution of the second script (or a third script) may also allow for visual analysis of insights gleaned through model-driven analysis of the data obtained from the database(s) 302 as further discussed below.

The development platform 210 can then implement the mutagenesis module 304 and optimization module 306 as part of an optimization operation. As mentioned above, the mutagenesis module 304 may be responsible for implementing a mutagenesis algorithm that introduces, to each candidate peptide, mutations across the interacting surface between that candidate peptide and its native interacting ligand or simulated interacting ligand. Generally, the mutagenesis algorithm implements single-point iterative mutagenesis without the use of machine learning, though the mutagenesis algorithm could use machine learning in some embodiments. Meanwhile, the optimization module 306 may be responsible for enhancing properties—like solubility, binding affinity, and delivery mechanism—based on an analysis of the mutated candidate peptides generated by the mutagenesis module 304. Again, the optimization module 306 may generally accomplish this without the use of machine learning. For example, the optimization module 306 may utilize thermodynamic modeling with or without iterative mutagenesis. Together, the mutagenesis module 304 and optimization module 306 allow the candidate peptides generated via the docking operation to be further analyzed to determine which, if any, have therapeutic applications and might be suitable for manufacture. For example, as part of the optimizing operation, the development platform 210 may consider aspects such as mutagenesis, secondary structure, energetics, solubility, delivery, or any combination thereof.

Other data could also be considered by the design module 218 as part of the optimization operation. Assume, for example, that the development platform 210 is tasked with developing a molecule for use in gene therapy. In such a scenario, the development platform may integrate physiochemical and other delivery data for nanoparticles, in an effort to design the molecule in a more targeted manner.

Thereafter, the development platform 210 can implement the synthesizing module 222 as part of a synthesizing operation. With the synthesizing module 222, the development platform 210 can ensure the workflow represents an end-to-end solution to developing peptides with therapeutic applications. As mentioned above, the synthesizing module 222 may be responsible for generating an output (e.g., in the form of synthesis instructions, peptide characteristics, etc.) that can be integrated into appropriate instrumentation (e.g., a peptide synthesizer) to enable end-to-end development of therapeutics. Because the synthesizing module 222 is part of the development platform 210, synthesis can be integrated into the workflow in a more meaningful manner, such that the result of performing the workflow can be a peptide or an output related to synthesis of the peptide.

In sum, the development platform 210 may implement an approach to designing molecules or molecule groups in which a given cell, tissue, or organ—or a set of cells, tissues, or organs—may be selected by a user and a list of candidates is automatically generated for targeting a specific site along the surface of a targeted biological substrate. The list of candidates could include polypeptides, polypeptoids, sugars, lipids, small molecules, polymer conjugates, polymers, recombinant proteins, nucleic acids, other synthetic ligands, and combinatorial or hybrid versions thereof, for example. Meanwhile, the targeted biological substrate may be a receptor, protein, glycoprotein, sugar, nucleic acid, lipid, small molecule, and combinatorial or hybrid versions thereof.

### A. Overview of Specificity Module

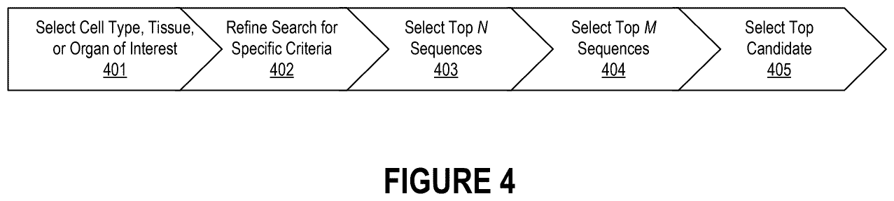

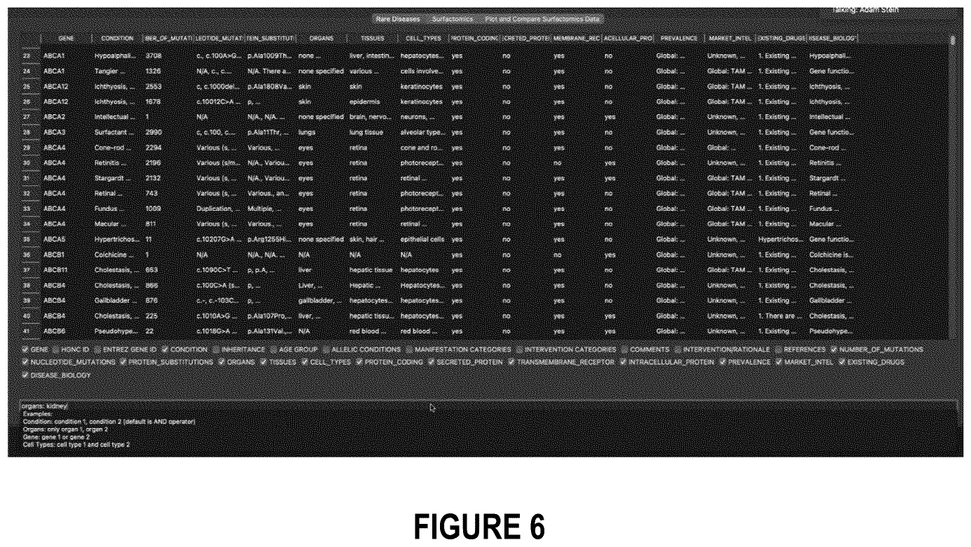

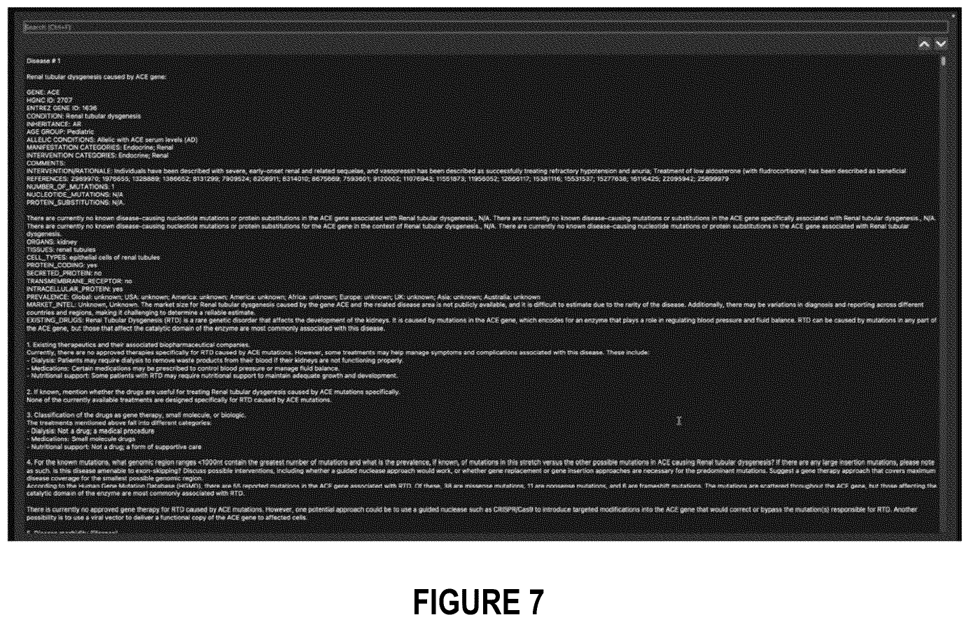

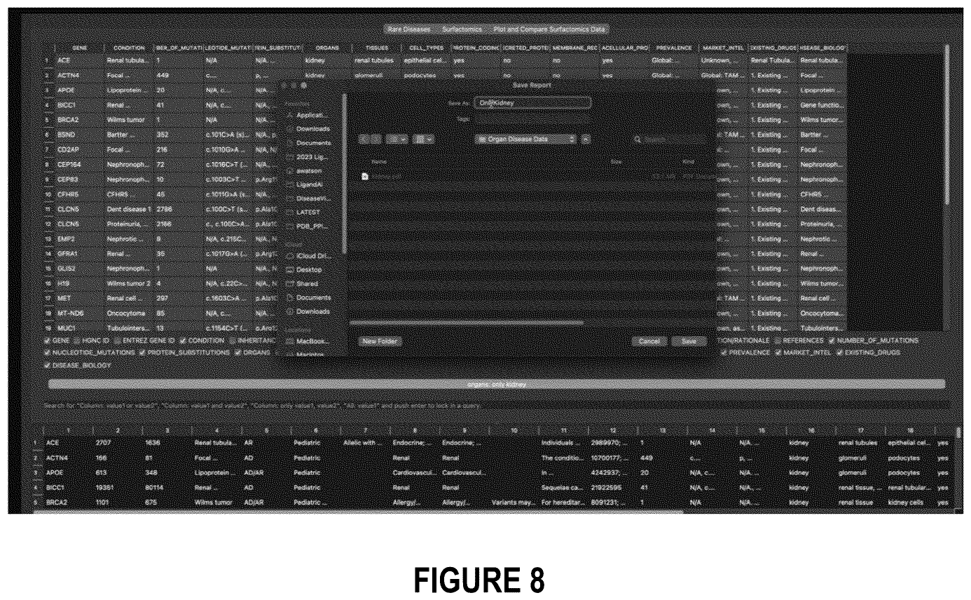

FIG. 4 includes a high-level illustration of a workflow that can be implemented by the specificity module 214. Initially, the specificity module 214 may receive input that is indicative of a selection, by a user, of a cell type, tissue, or organ of interest via an interface (step 401). FIG. 5 includes an example of an interface through which a user can interact with the specificity module 214, while FIG. 6 includes an example of an interface through which the user is able to select the cell type, tissue, or organ of interest. Here, a selection of the kidney has been made. Upon receiving the input, the specificity module 214 may review data that is associated with the selected cell type, tissue, or organ and is available to the development platform 210 (e.g., obtained from one or more databases 228) to produce search results. FIG. 7 includes an example of an interface that shows a typical output for an organ search conducted by the specificity module 214, while FIG. 8 shows how the user may be able to save the search results produced by the specificity module 214.

In some embodiments, the interfaces shown in FIGS. 5-6 allow users to enter multiple queries. For example, a user may opt to include or disinclude various cells, tissues, or organs. Therefore, one could find the most common and most expressed surfaceome of multiple organs (e.g., the kidney and lung), or even separately look at different tissues (e.g., the medulla of the kidney versus the cortex of the kidney). Depending on the intended application, one or more targets could be identified (e.g., selected).

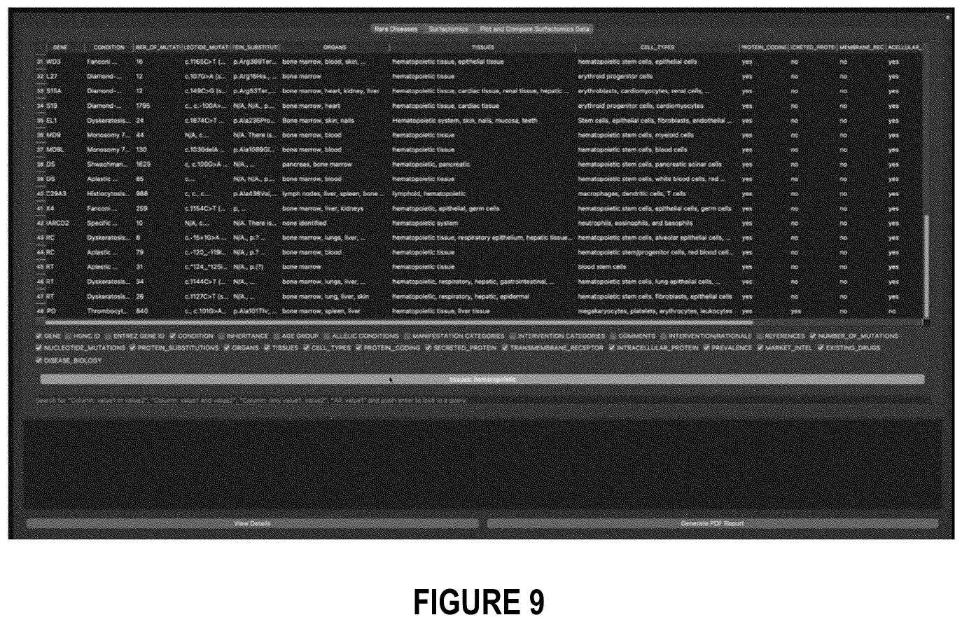

After the search is completed by the specificity module 214—here, for diseases and associated information that affect the kidney—the user can take the search results and further refine the search for more specific criteria (step 402). For example, if the user initially selects an organ or set of organs, then the user may select a tissue or cell type of interest. Other examples of criteria can be seen in FIG. 9. FIG. 9 includes an example of an interface that shows how the user can refine the search results to more specific interactome patterns.

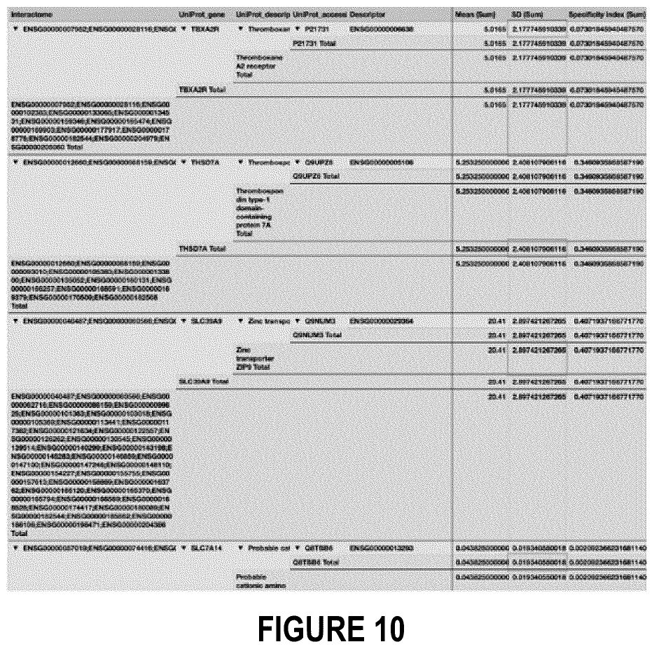

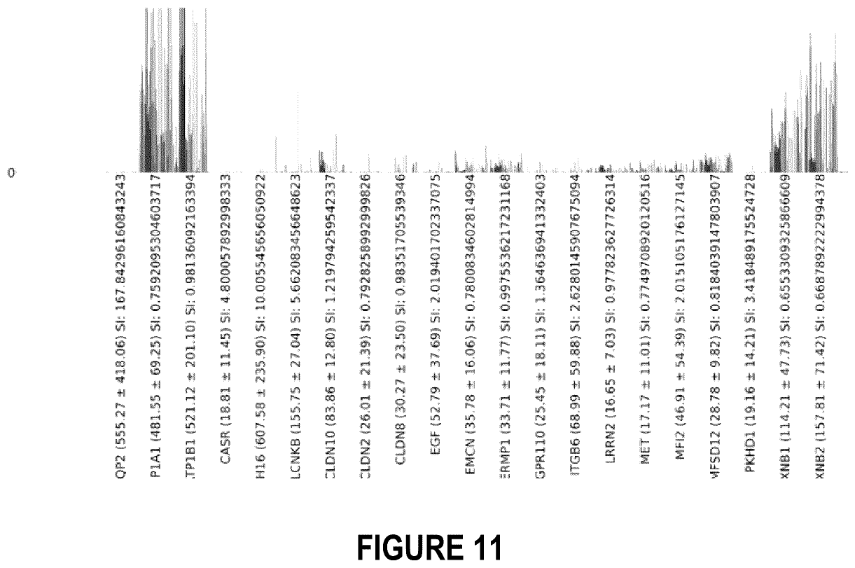

Then, the specificity module 214 can implement one or more filtering operations. In FIG. 4, the workflow includes two filtering operations. First, the specificity module 214 parses the search results to identify the top n sequences (step 403), where n is an integer value. Second, the specificity module 214 parses the top n sequences to identify the top m sequences (step 404), where m is an integer value that is less than N. For example, the specificity module 214 may initially filter the search results to the top 500 cell-surface sequences and then to the top 100 sequences that have a subsequent desired characteristic. In some instances, the further-filtered m sequences are sequences where interactomics data is known (e.g., protein A binding to protein B), but docking and binding data do not necessarily exist. As another example, the specificity module may initially filter the search results to the top 2,886 sequences and then to the top 100 sequences. As another example, the specificity module 214 may initially filter the search results to the top 280 sequences and then to the top 50 sequences. As another example, the specificity module may initially filter the search results to the top 150 sequences and then to the top 10 sequences. The values for n and m may depend on various factors, including the total number of raw search results or refined search results, as well as the computational resources available to the development platform 210. FIG. 10 shows how the refined search results could be initially filtered and clustered to the top n sequences, where n equals 280. FIG. 11 shows how the top n sequences can then be filtered and clustered to the top m sequences, where m equals 50.

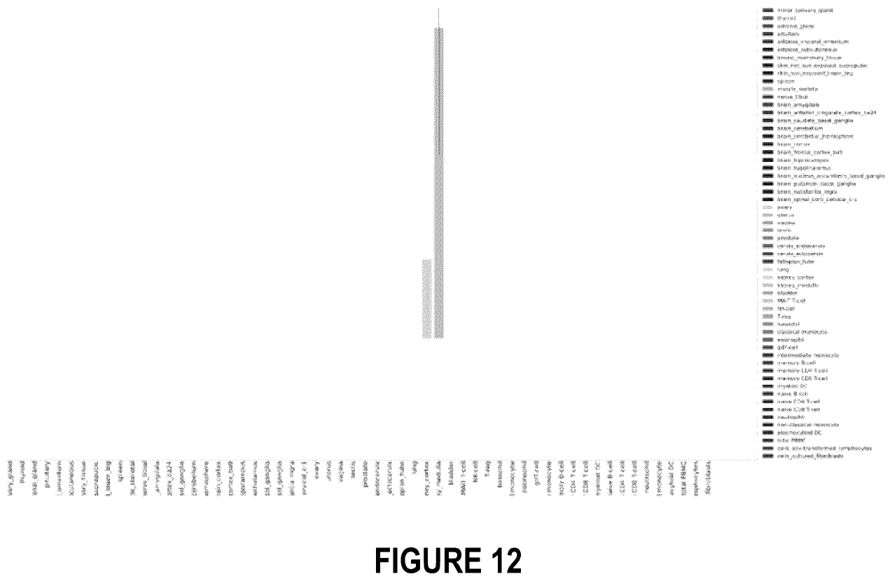

The specificity module 214 can then select the top candidate based on an analysis of the remaining top m sequences (step 405). FIG. 12 shows how the top candidate (i.e., the UMOD gene) can be selected from among the remaining top m sequences. The top candidate may be identified, through analysis of the top m sequences, as the most selective sequence by the specificity module 214. For example, the specificity module 214 may calculate selectivity indices for the top m sequences and then select the sequence having the highest selectivity index as the top candidate. The selectivity index is a measure of the likelihood that a specific protein will bind to another protein or a receptor along the surface of a cell, tissue, organ, etc. Using a filtering approach, the specificity module 214 can determine the proteins (and corresponding genes) that have never been known to bind to other proteins, cells, tissues, organs, etc. In other instances, binding may be known but the interactomics data specifically enables the discovery of novel binding agents.

### B. Overview of Docking Module

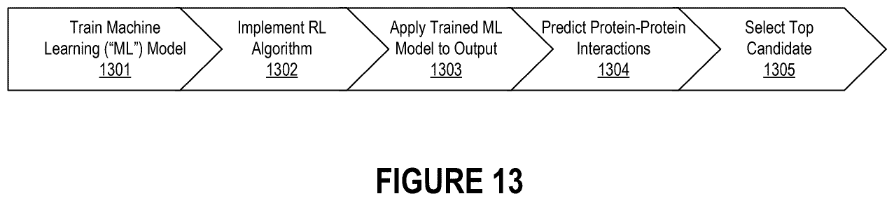

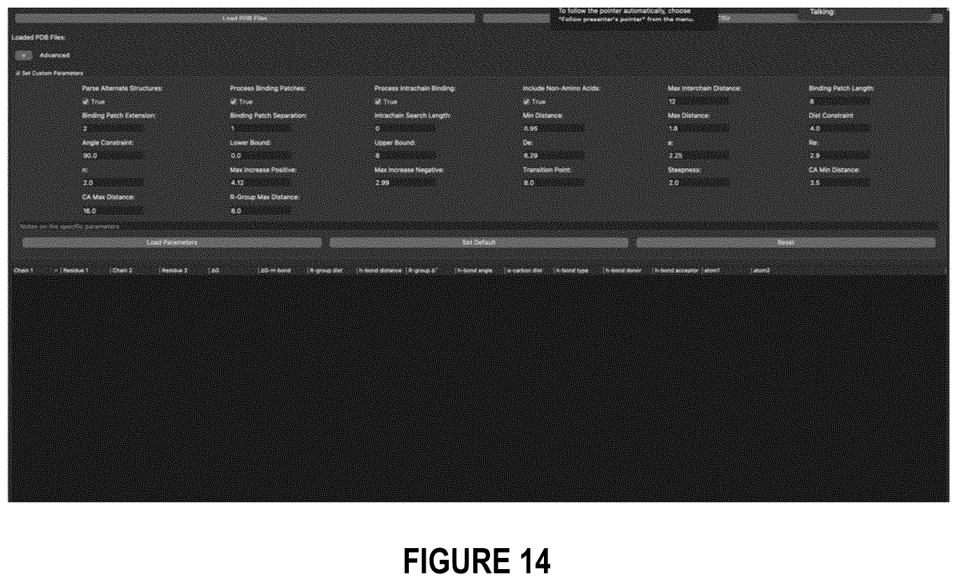

FIG. 13 includes a high-level illustration of a workflow that can be implemented by the docking module 216. FIG. 14 includes an example of an interface through which a user can interact with the docking module 216. As mentioned above, the docking module 216 may be responsible for predicting the docking interfaces for some subset of the candidate peptides generated by the specificity module 214. For example, the docking module 216 may predict the optimal docking interface for only the top candidate peptide selected by the specificity module 214. Through the interface shown in FIG. 14, the user can specify parameters that govern the predicting. Said another way, the user may be able to adjust parameters that are available to refine the calculations performed by the docking module 216 through the interface shown in FIG. 14. These parameters can include binding patch extension, binding patch separation, angle constraint, intrachain search length, minimum distance, maximum distance, steepness, binding patch length, etc. Accordingly, the docking module 216 may permit the user to customize the predicting by specifying one or more parameters that indicate, specify, or limit how the candidate peptide(s) should dock.

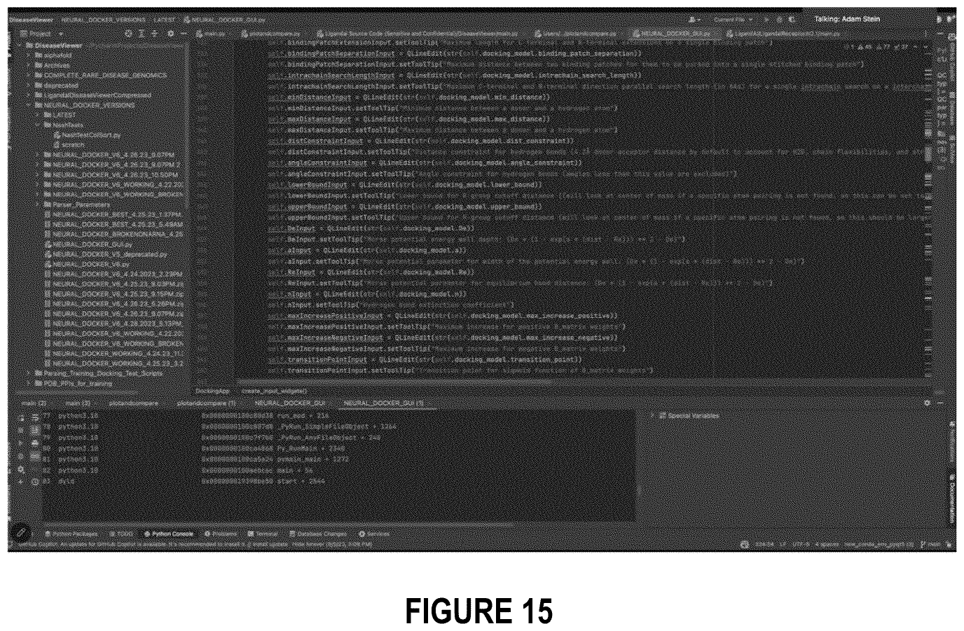

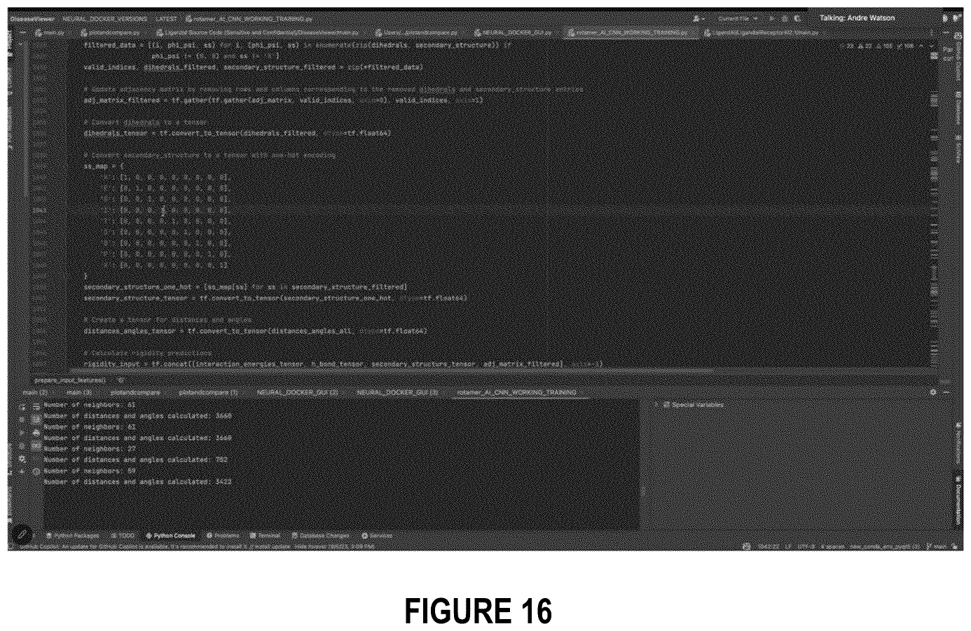

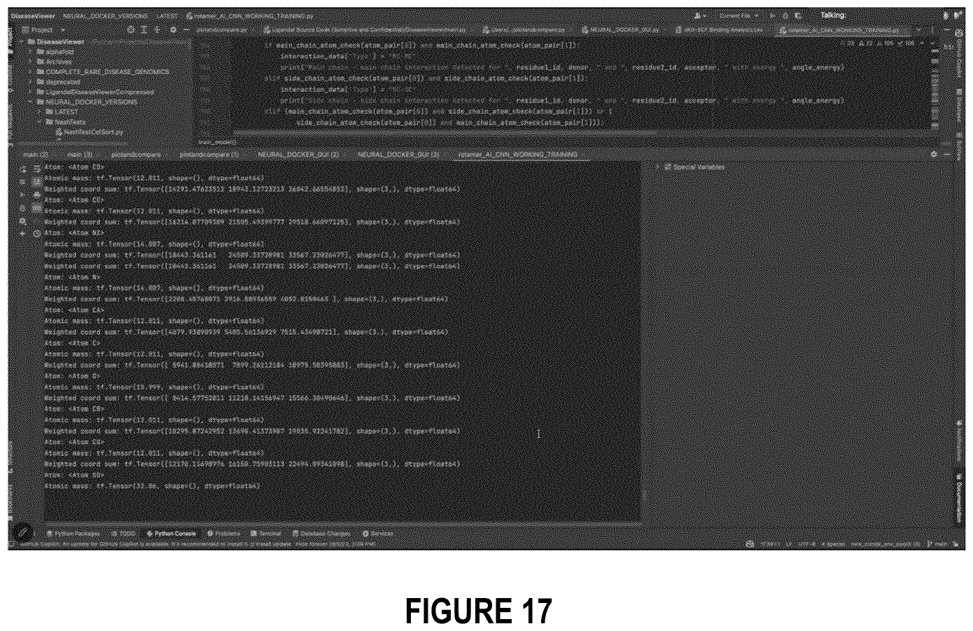

FIG. 15 includes explanations of parameters for controlling the operations that are performed by the docking module 216. FIG. 16 shows an example of a tensor matrix that may be used by the docking module 216, while FIG. 17 shows how the docking module 216 may use the tensor matrix in operation. In FIG. 17, the docking module 216 is implementing a tensor operator that calculates positional coordinates using a weighting system. In machine learning, data can be organized in a multidimensional array that is commonly referred to as a “tensor matrix” or simply “tensor.” This multidimensional array—which has a specific shape and dimensionality—can be used to represent input data to which the docking module 216 can apply a machine learning model and output data that is produced by the machine learning model. The tensor can include any data that is known at the time of loading a structure or set of structures, and may also be a data output (e.g., produced by the machine learning model). In some embodiments, the tensors represent 3D coordinates, secondary structures of one or more amino acids as corresponds to the nearby sequences (e.g., as a primary structure or as a voxel index), the thermodynamics of possible states of interaction between chains or within a chain, the rotation and translation of a chain, the presence of dihedrals or rotamers, or any multi-dimensional dataset associated with each atom, individual mer, or set of atoms or mers comprising one or more simulated molecules with or without an interaction component with another molecule. Note that, in some embodiments, the docking module 216 does not necessarily need to use machine learning. Instead, the docking module 216 may process interactions without using machine learning and either with or without converting the underlying data components into tensors.

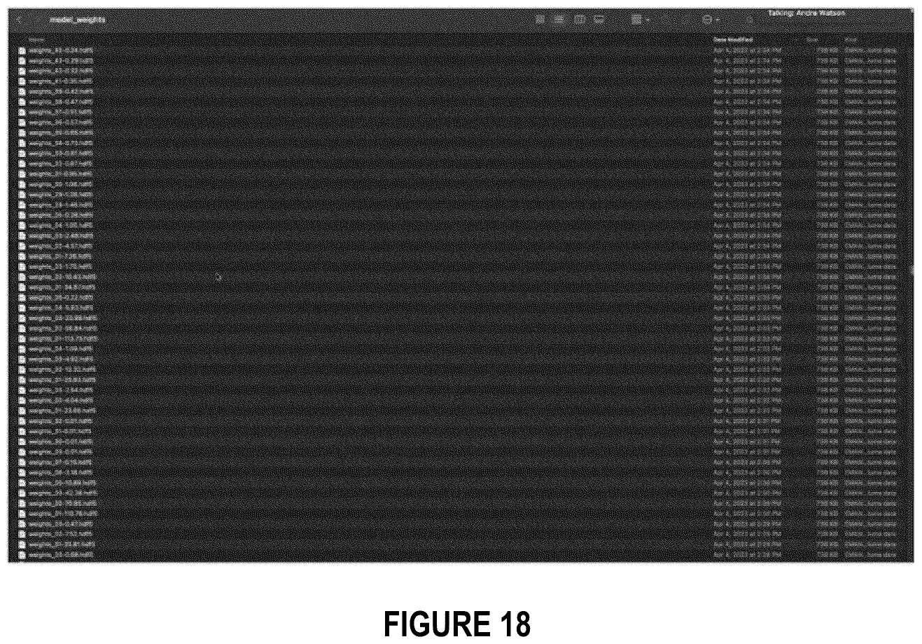

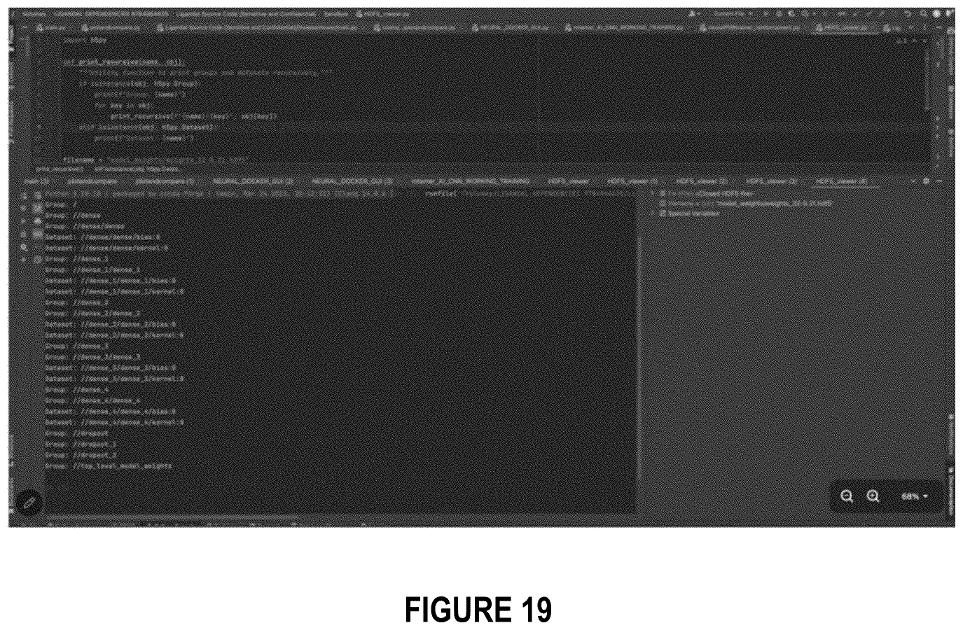

As mentioned above, the docking module 216 can use a weighting system to determine optimal interactions. FIG. 18 shows an example of an output that may be produced by the weighting system. While the weights output by the weighting system may be useful to the docking module 216 (and development platform 210 more generally), these weights may not be readily understandable by users. Accordingly, the development platform 210 may support a visual tool—written in a programming language such as Python or Java—that creates visualizations of the outputs produced by the weighting system. For example, the visual tool may be designed to present a visualization that is representative of the content of an Hierarchical Data Format (“HDF”) file or analogous weight-containing file produced by the weighting system as output. FIG. 19 illustrates how a user may be able to access the visual tool, and the starting data that is subsequently processed for interactions with the option of being fed into one or more neural network or deep learning approaches.

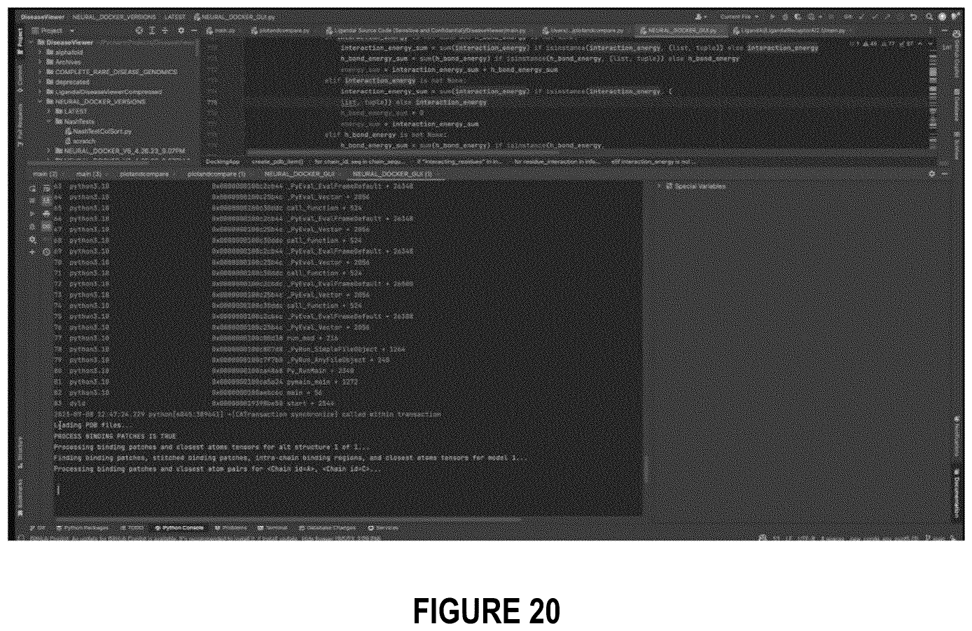

With the positional coordinates, the docking module 216 can attempt to better understand the likelihood of docking between the top candidate and other proteins, molecules, cells, tissues, organs, etc., by utilizing a voxel index based parsing methodology, where each voxel's input and output parameters may be fed into a neural network. FIG. 20 illustrates how the docking module 216 could process binding patches between the top candidate (here, a first protein) and a target (here, a second protein) in order to identify an optimal docking interface. As part of the processing, the docking module 216 may attempt to find docking interfaces, stitched docking interfaces, intrachain binding regions, and closest atom tensors for the target. The docking module 216 may also utilize voxel-indexing based approaches, or distance constrained relative positions between inter-chain (e.g., between molecule A and molecule B) and intra-chain (e.g., within molecule A or molecule B) interactions in order to predictively assess interactions; it may also be trained on empirically evaluated structures of two or more bound molecules in order to subsequently be able to predict interactions between two or more molecules that have not been docked.

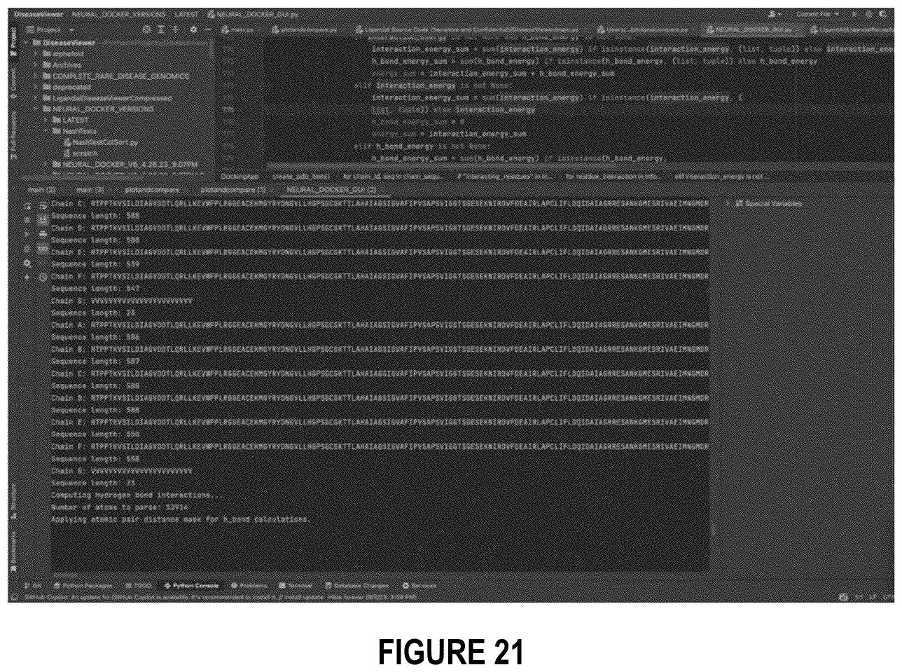

An important part of the predicting may involve calculating hydrogen bonds, hydrophobic interactions, van der Waals forces, hydrophilic interactions, electrostatic interactions, and other interactions between each candidate peptide and various docking interfaces. To accomplish this, the docking module 216 may implement a computer program—called an “interaction energy calculator”—that calculates all of the possible hydrogen bonds and other sidechain interactions of interest (e.g., non-hydrogen-bond interactions such as hydrophobic interactions) between each candidate peptide and various docking interfaces. Using docking surface information, the docking module 216 may be able to optimize the hydrogen bond distance and other intramolecular distances, as well as optionally optimizing the rotamers and chain rotations and translations, between docking interfaces. FIG. 21 shows an example of an interaction energy calculator that can be used by the docking module 216.

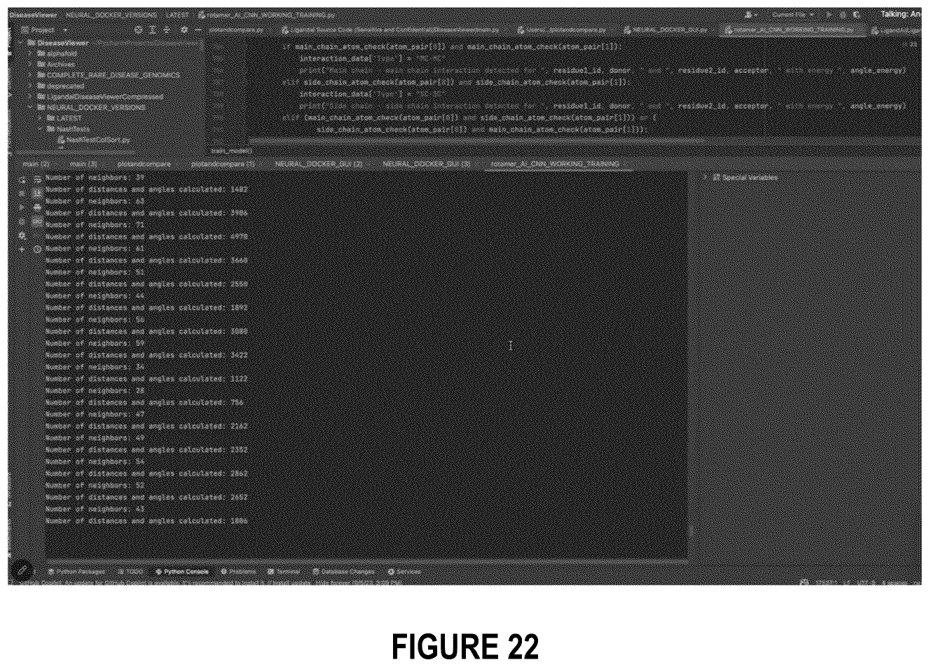

Determining nearest neighbors may also be an important part of the predicting. Using docking surface information and hydrogen bond information computed by the interaction energy calculator, the docking module 216 may be able to optimize nearest neighbor calculations. For each docking interface, the docking module 216 may not only identify the number of neighbors but also the number of distances and angles calculated between inter-chain and intra-chain interacting and non-interacting atoms. With this information, the docking module 216 can better determine which docking interfaces are most appropriate for each candidate peptide. FIG. 22 shows an example of a nearest neighbor calculator that can be used by the docking module 216.

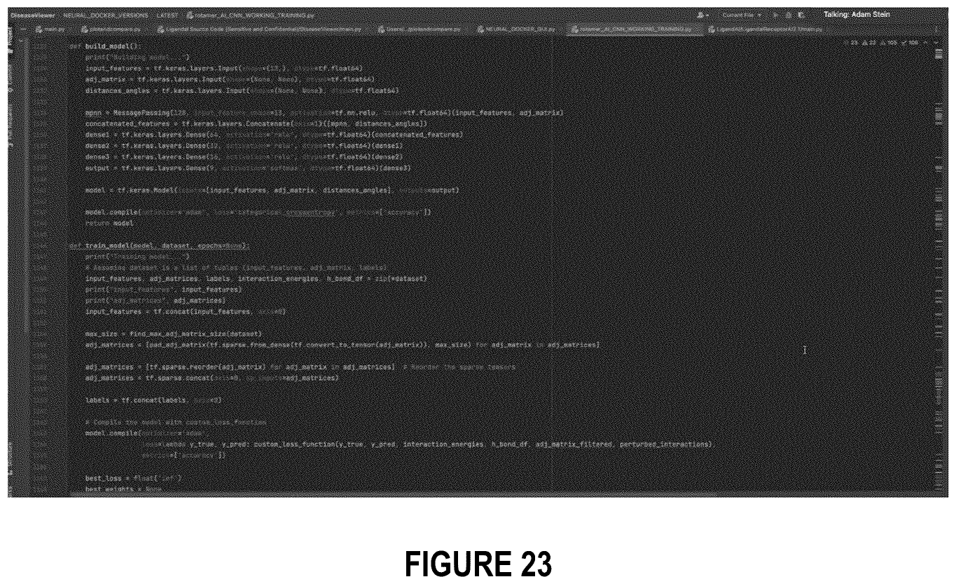

As discussed above, the docking module 216 may be responsible for applying a machine learning model to an output that is produced by the specificity module 214—like the top candidate selected in step 305 of FIG. 3—and additional data. The docking module 216 may also be responsible for training the machine learning model (step 1301). FIG. 23 includes example code for training the machine learning model in accordance with some embodiments. Specifically, FIG. 23 illustrates how the machine learning model can be trained on a dataset—commonly called a “training dataset”—comprised of tuples. Each tuple may include one or more attributes, a matrix of values, and a label that indicates whether the one or more attributes are indicative of an outcome. At a high level, the process of training a machine learning model involves providing a machine learning algorithm with a training dataset from which to learn relationships and another dataset—commonly called a “validating dataset”—from which to validate the learned relationships. The machine learning algorithm tries to discover patterns in the training dataset that relate the attributes and labels and then outputs the machine learning model that captures these patterns. These datasets may immediately feed into a machine learning model, or may be stored in a database for subsequent processing by a machine learning model or conventional structural biology and rational design approaches.

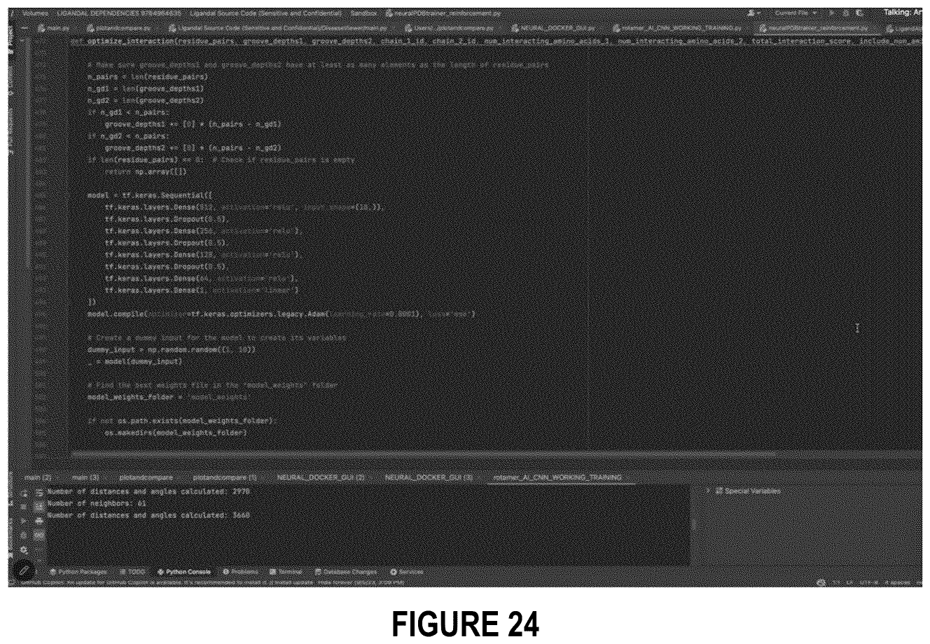

In some instances, reinforcement of the machine learning model (and, more specifically, its learnings) is helpful. This is especially true where the number of actions and states of interest is in the hundreds or thousands, millions, billions, or trillions. Simply put, reinforcement may be helpful in establishing that the patterns between the attributes and labels in the training dataset were appropriately learned. One approach to reinforcing a machine learning model involves using an RL algorithm. Accordingly, the docking module 216 may implement an RL algorithm (step 1302) for reinforcement of the machine learning model. FIG. 24 includes example code for reinforcement of the machine learning model.

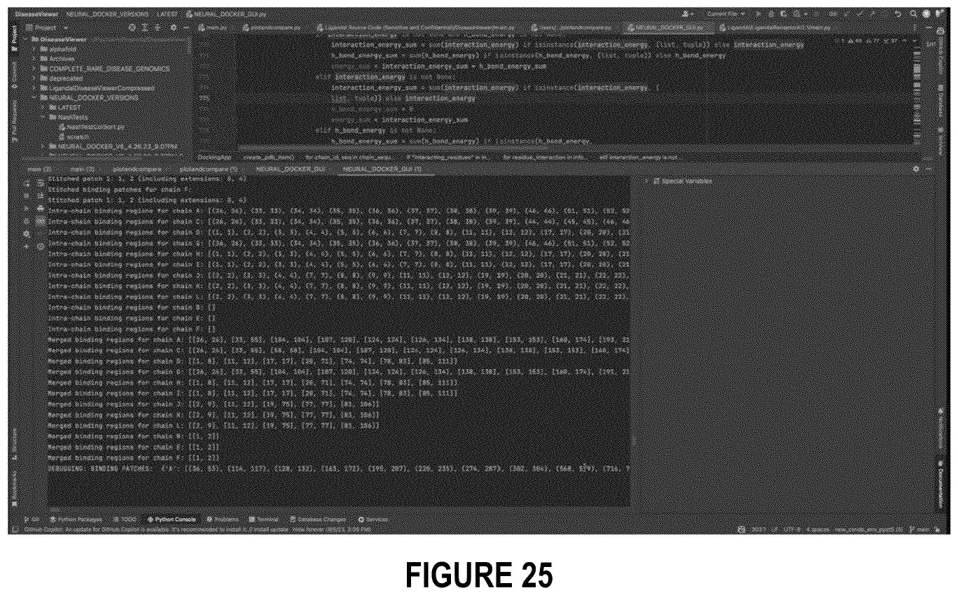

In operation, the docking module 216 may apply the machine learning model to an output that is produced by the specificity module 214 (step 1303)—like the top candidate selected in step 305 of FIG. 3. For the top candidate, the machine learning model can produce indications of locations of different docking interfaces, stitched docking interfaces, intrachain binding regions, closest atom tensors, or any combination thereof. FIG. 25 shows an example of an initial output that can be generated by the docking module 216. As can be observed in FIG. 25, the stitching approach is able to reduce the original 3D structures into regions that have interactions between one another, whether those interactions are within the chain or between chains.

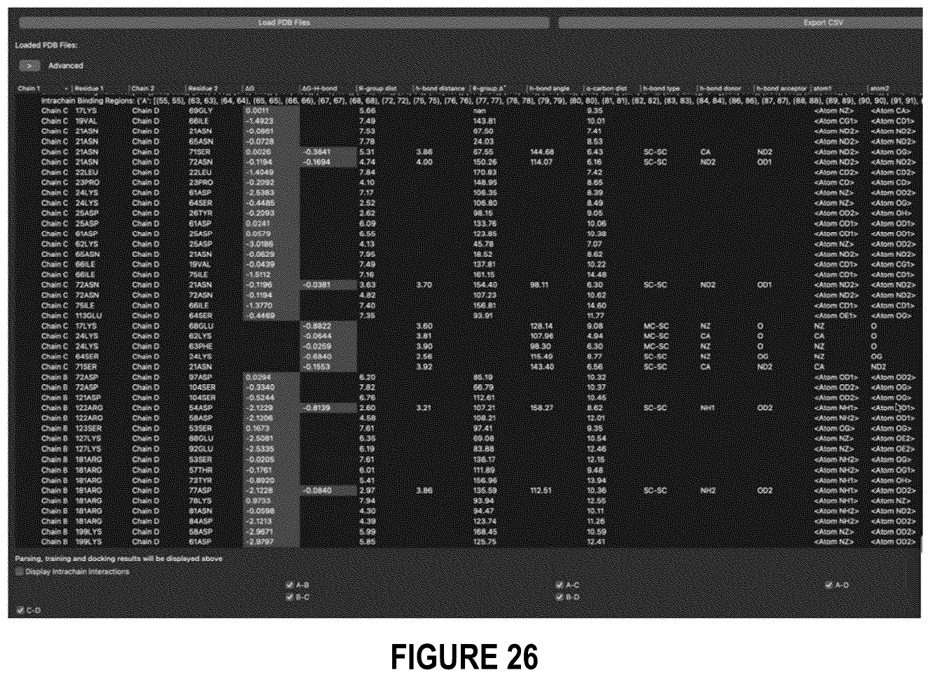

Then, the docking module 216 can predict protein-protein interactions along and near the surfaces of a candidate of interest (step 1304). As mentioned above, the candidate of interest could be an organ, tissue, or cell type. To predict protein-protein interactions, the docking module 216 may calculate free energy as shown in FIG. 26. Specifically, FIG. 26 shows the results of an “run” by the docking module 216. If intrachain interactions are not displayed, the thermodynamic interactions within each respective chain may not be shown; only the interactions between chains may be shown as a result. However, more thermodynamically favorable interactions may be visually distinguishable from less thermodynamically favorable interactions. In FIG. 26, colors are used to visually indicate good energetics and bad energetics around interacting residues. Such an approach to flagging insights into energetics allows the results to be readily understood by users with different levels of expertise in development and experience with the development platform 210. These visually distinguishable regions may form the basis for the mutagenesis algorithm finding optimized stretches of sequences within each patch of stitched binding patch, which the user can alter the parameters of depending on whether she wants to find 5-mer, 10-mer, 20-mer, or other sequences or regions that can be a basis for peptide discovery and design.

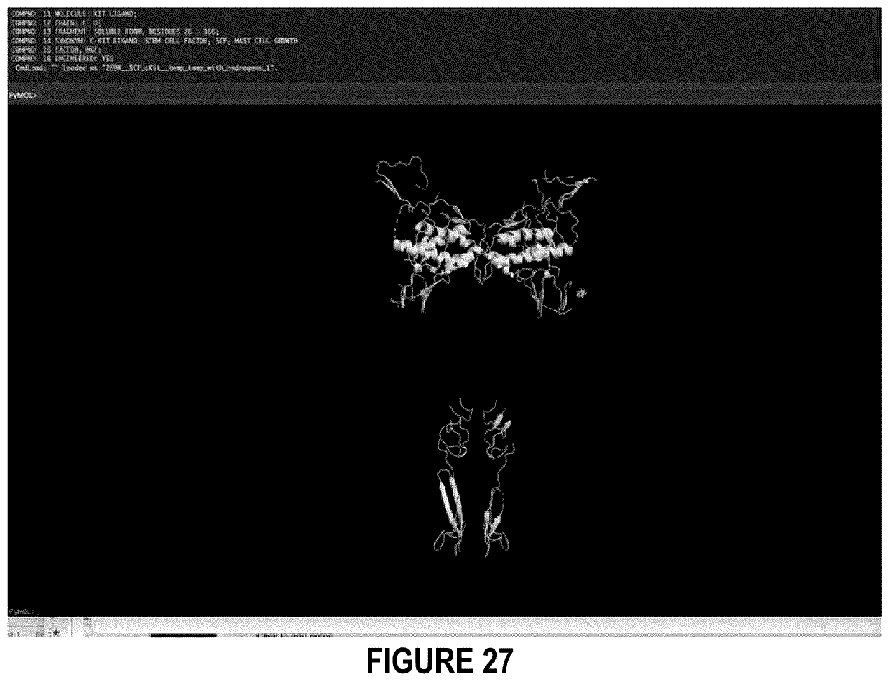

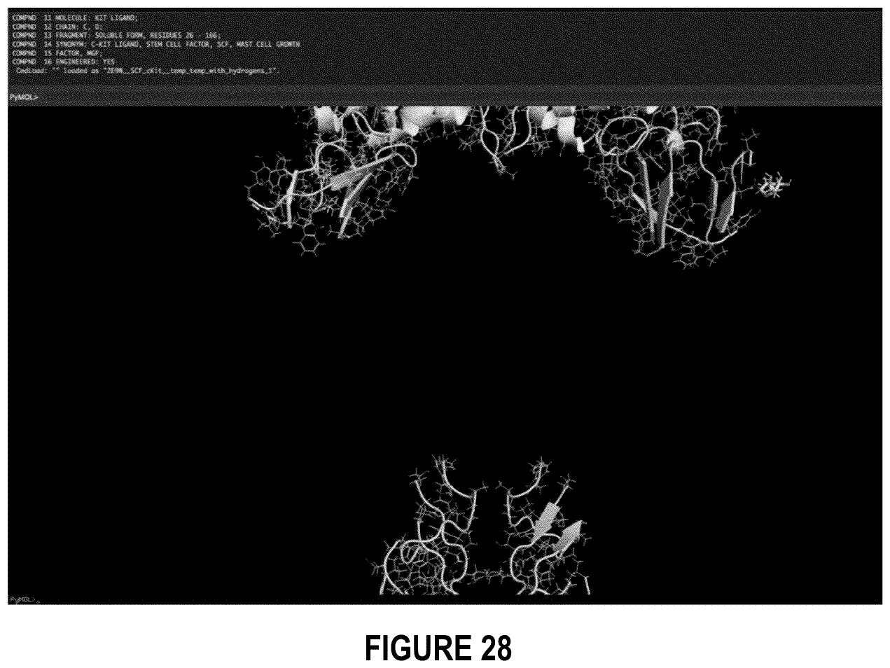

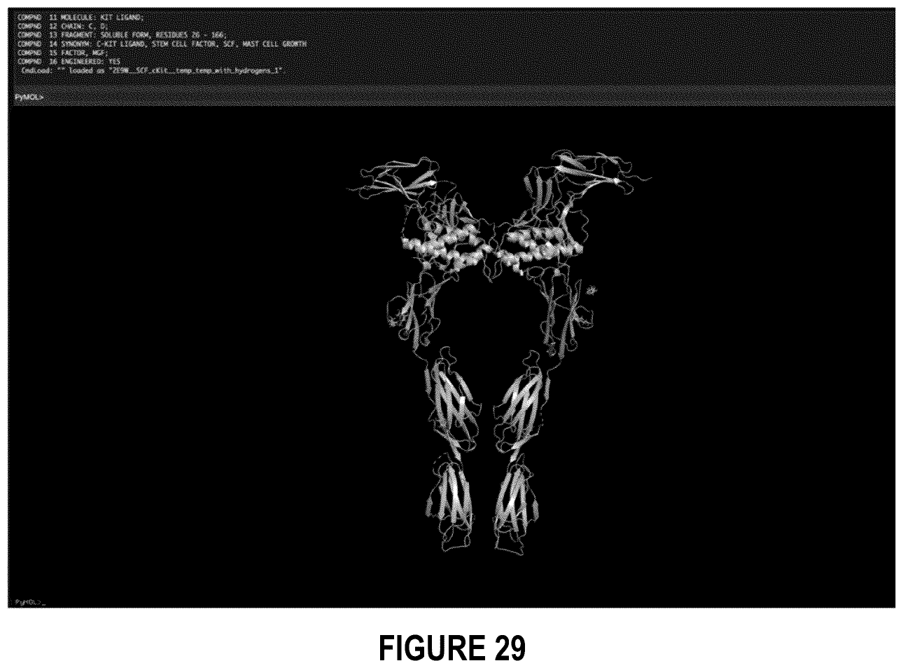

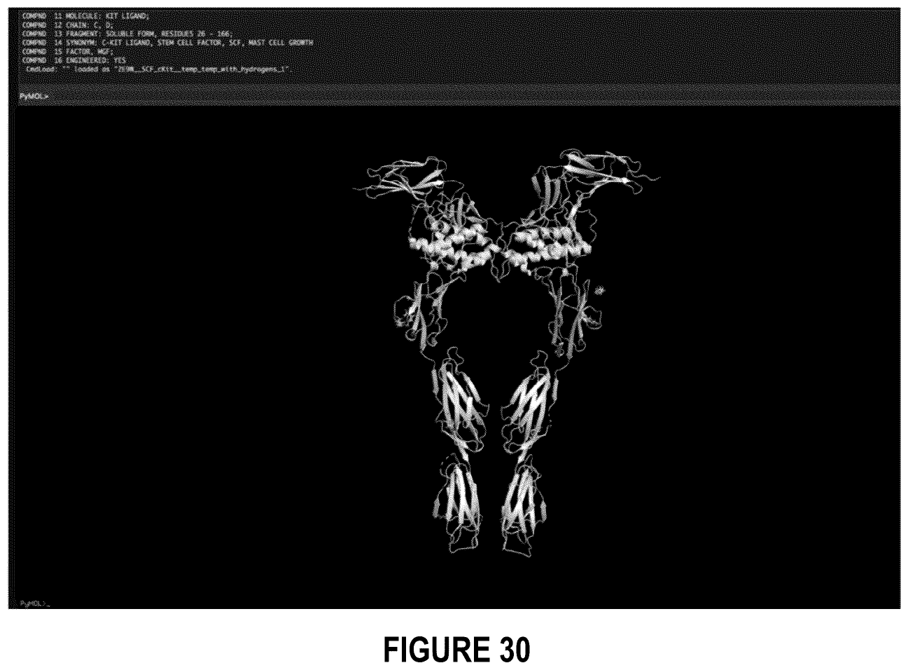

Based on the protein-protein interactions, the docking module 216 can then predict, compute, or otherwise produce at least one 3D structural model. Assume, for example, that the development platform 210 is interested in developing a peptide-based ligand. In such a scenario, the docking module 216 can produce a structural model for a protein that is representative of, or included in, the ligand. FIG. 27 shows an example of a structural model produced for a protein. To produce the structural model, the docking module 216 may “trim” the target into parts that contain surface interactions with a binding partner or intramolecularly. Further, the docking module 216 can produce a structural model for the protein with calculated hydrogen bonds and other thermodynamic interactivity. FIG. 28 shows an example of another structural model produced for the protein but with hydrogens appended thereto. This version of the protein may be the one upon which the peptide is designed. Moreover, the docking module 216 can overlay the structural model generated for the protein—as shown in FIG. 28—on another structural model that is representative of the known crystal structure of the protein. Such a visualization allows the user to readily compare the structural model as generated by the docking module 216 with the known structure of the protein. FIG. 29 shows an example of a structural model for the crystal structure of the protein, while FIG. 30 illustrates how the docking module 216 can overlay its generated structural model on the known structure of the protein.

### C. Overview of Design Module

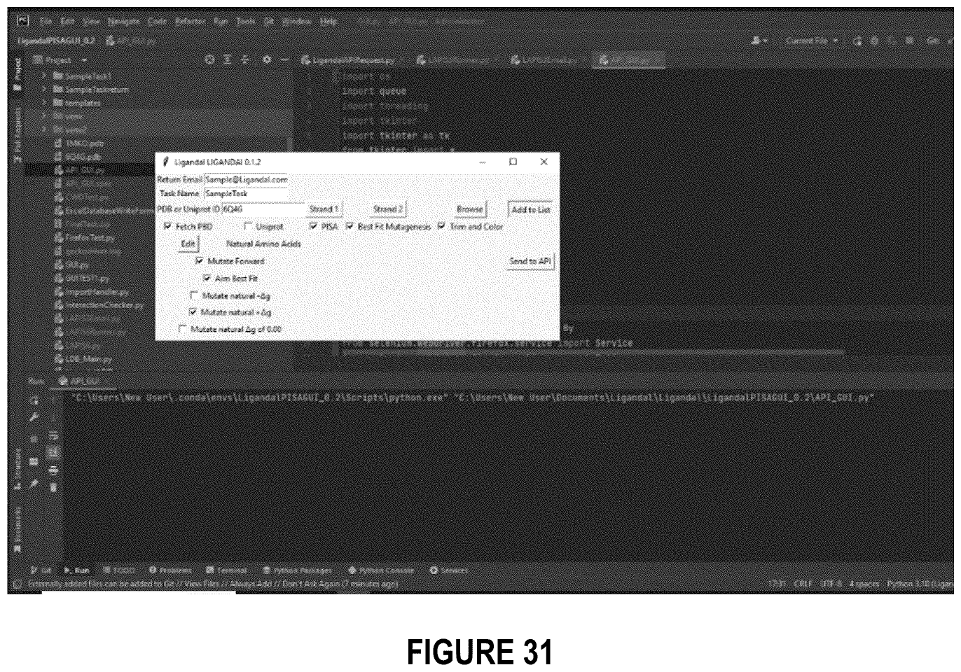

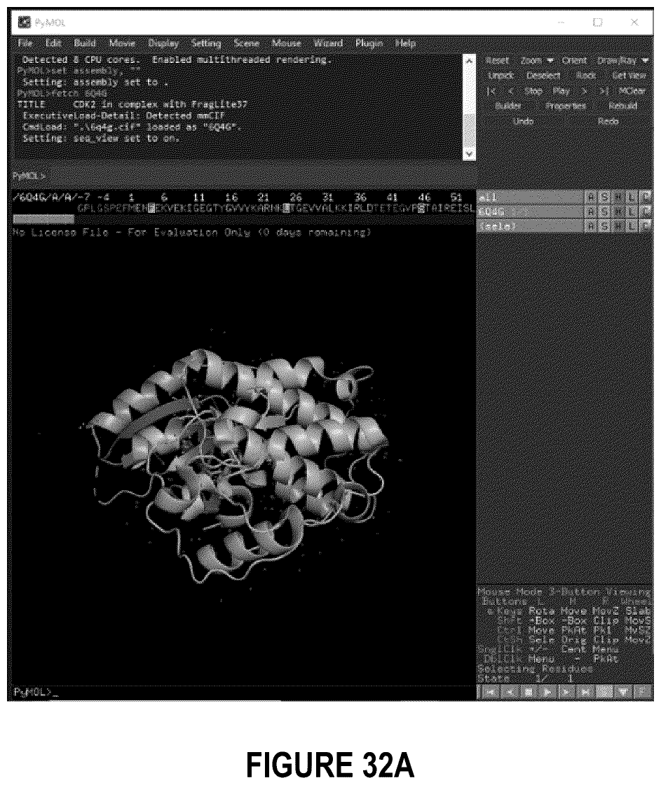

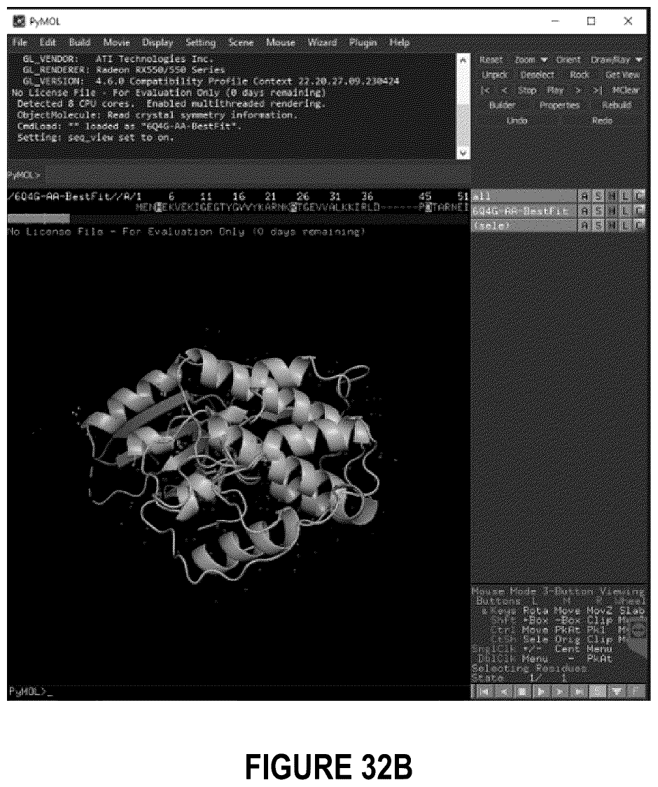

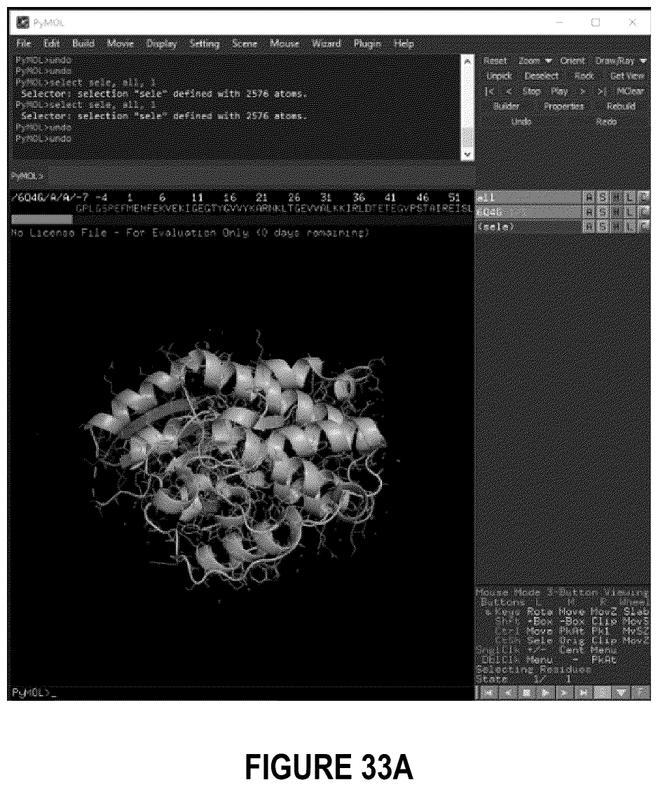

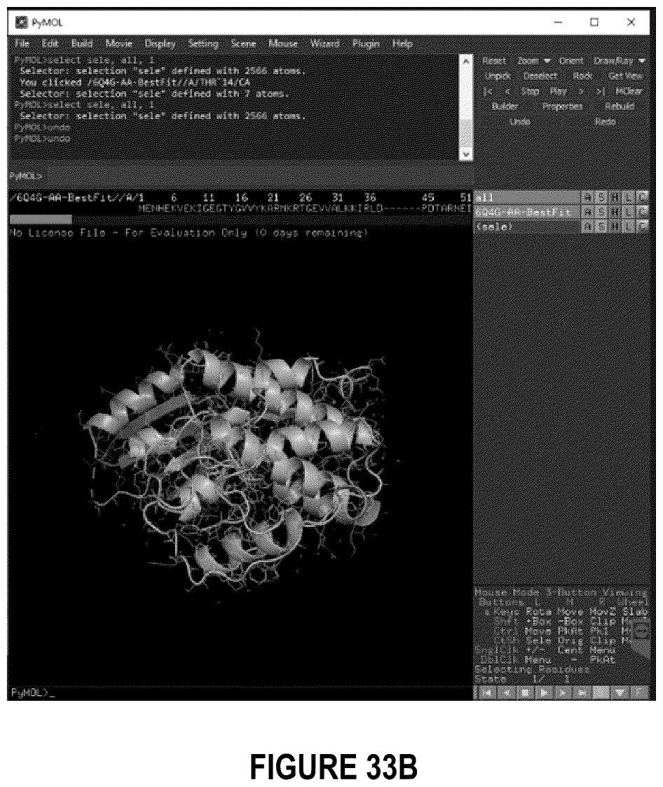

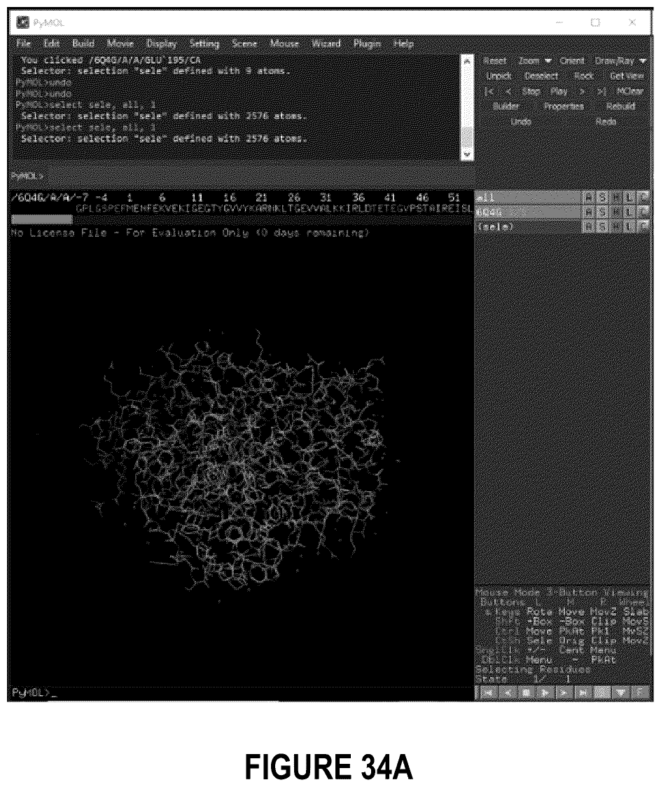

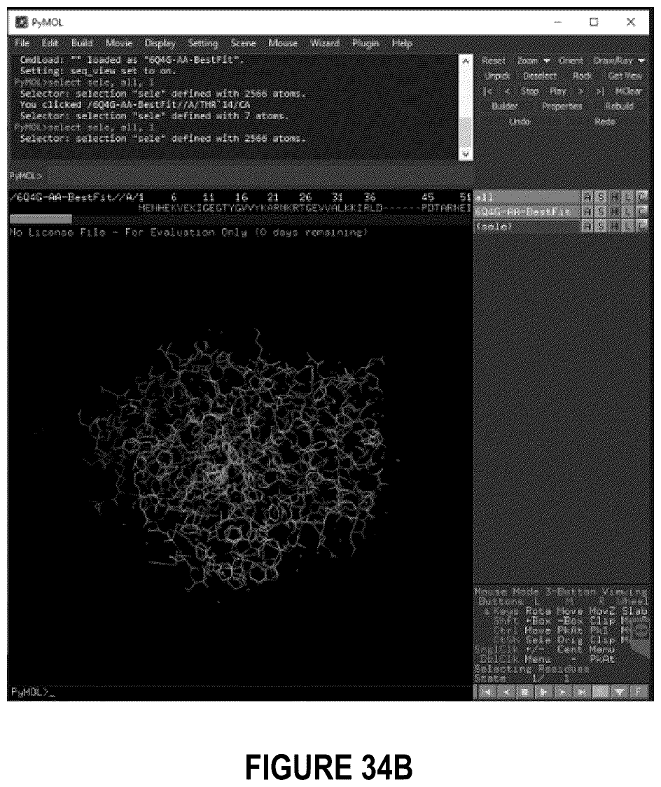

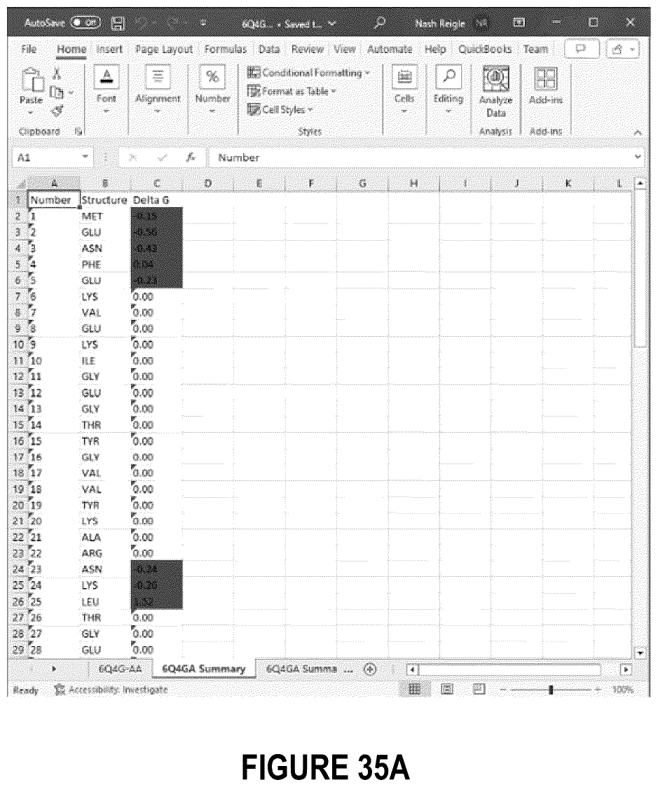

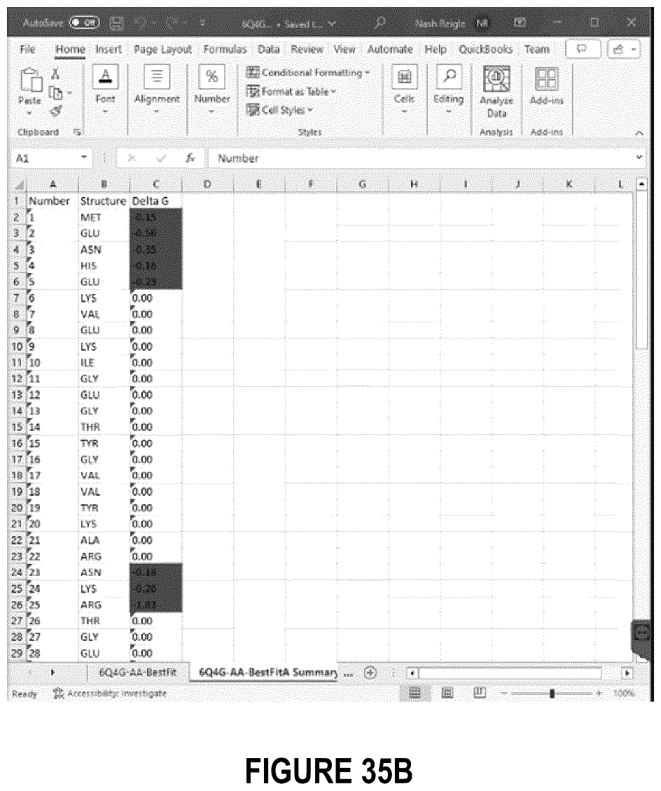

As mentioned above, the design module 218 may implement a mutagenesis module 304 and optimization module 306 as part of an optimization operation in which the top candidate identified as part of the docking operation is further optimized. The mutagenesis module 304 may be responsible for implementing a mutagenesis algorithm that introduces, to each candidate peptide, mutations across the interacting surface between that candidate peptide and its native interacting ligand or simulated interacting ligand. The interactions do not necessarily need to be between peptides, as the hydrogen bonds and other interactions are assessed on an atom-by-atom basis regardless of whether the one or more interacting molecules are proteins, DNA, RNA, sugars, or other molecules. FIG. 31 shows an example of an interface through which a user is able to select a peptide or collection of peptides for which mutagenesis is to be performed. Aspects of the mutagenesis—like whether to mutate forward or mutate naturally with an emphasis on adding, losing, or maintaining free energy—can be specified through this interface. Mutagenesis can be initiated by selecting the graphical element labeled “Send to API.” FIGS. 32A-B include examples of visualizations that may be produced (e.g., by the visualization module 220) based on outputs produced by the mutagenesis algorithm or analyses of the outputs by the design module 218. In FIGS. 32A-B, for example, a candidate (here, the CDK2 protein) to which mutations are introduced is shown in one color while indications of the mutations are shown in another color. In FIGS. 33A-B, mutated structure files, as generated by the mutagenesis module 304, with mutated residues determined by the mutagenesis algorithm, while FIGS. 34A-B show only the mutated residues determined by the mutagenesis algorithm. FIGS. 35A-B include examples of data structures (here, spreadsheets) in which the outcome of the mutagenesis is documented. Specifically, the leftmost data structure includes a summary of mutagenesis results in terms of effect on free energy while the rightmost data structure includes a summary of best fit mutagenesis results in terms of effect on free energy. As shown in FIGS. 35A-B, mutations having a desired impact (e.g., decreasing free energy) may be visually highlighted for the user. In some embodiments, mutations having an undesired impact (e.g., increasing free energy) may also be visually highlighted for the user.

Meanwhile, the optimization module 306 may be responsible for enhancing properties—like solubility, binding affinity, and delivery mechanism—based on an analysis of the mutated candidate peptides generated by the mutagenesis module 304. Together, the mutagenesis module 304 and optimization module 306 allow the candidate peptides generated via the docking operation to be further analyzed to determine which, if any, have therapeutic applications and might be suitable for manufacture. For example, as part of the optimizing operation, the development platform 210 may consider aspects such as mutagenesis, secondary structure, energetics, solubility, delivery, or any combination thereof.

Approaches to Generating Compounds with Therapeutic Applications

Historically, precision genetic medicine has focused on the use of adeno-associated viral (“AAV”) vectors or other non-viral delivery carriers, such as for delivering CRISPR-Cas9, mRNA, DNA, or other modalities to develop therapeutic applications. However, such an approach tends to be costly in terms of time, dollars, and resources (e.g., labor, cost of goods, development costs), slowing development. To facilitate more rapid development of therapeutics, the development platform 210 was created. The development platform 210 can develop compounds—like peptides, for example—that block proteins from binding to the appropriate receptor both in vivo and ex vivo, or mimic an interaction in order to bind to an appropriate receptor both in vivo and ex vivo. As a result, compounds with therapeutic potential can be generated via a universal procedure through the use of artificial intelligence, protein, and macromolecular structure prediction, and structural analysis thereof.

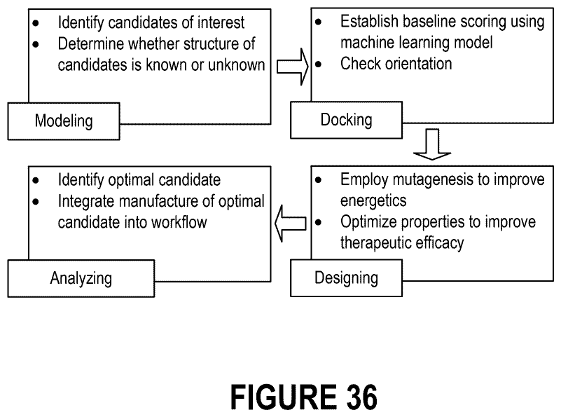

FIG. 36 includes a high-level illustration of a process by which a compound can be developed. As shown in FIG. 36, the process can include four stages, namely, (i) modeling, (ii) docking, (iii) designing, and (iv) analyzing. Each of these stages may involve separate modules as discussed above.

The modeling stage can vary depending on the nature of the compound being developed. Assume, for example, that the development platform 210 is tasked with developing a peptide-based ligand to bind to a receptor. There are three scenarios of interest, namely, (i) where ligand structure is known but receptor structure is unknown, (ii) where receptor structure is known but ligand structure is unknown, and (ii) where ligand and receptor structures are unknown. For each structure that is unknown, the development platform 210 can integrate available databases containing information that is needed for modeling and then obtain (e.g., generate or identify) a known structural model for docking. Information can be sequentially threaded through, or applied against, the known structural model, and a structural model for the unknown structure can then be built to reflect discovered properties.

The structural models generated in the modeling stage can then be validated for alignment and/or orientation to determine the theoretical structure for the ligand-receptor complex. Structural alignment may involve clustering analysis. Meanwhile, structural orientation may be determined via multiple independently executable algorithms that compare the structural models of the ligand and receptor. The ligand-receptor complex that is most statistically relevant may be used to design the peptide.

Using Proteome Integral Solubility Alteration (“PISA”) or proprietary software that carries enhanced prediction of atomic interactions between two or more interacting chains, the development platform 210 can calculate the surface area of the ligand-receptor interface and look for ligand residues that are important for binding the receptor. The residues that are determined to be important may be identified as unchangeable. Then, the development platform 210 can calculate the free energy values for all residues at the interface. Residues with negative and zero free energy values may be left undisturbed in the initial design. However, residues could be labeled or colored according to their free energy values as shown in FIGS. 35A-B. After free energy values are calculated and assigned a label or color, stretches of secondary structure (e.g., α-helix, β-sheet, random coil) can be defined that contain both the essential binding residues and boundaries to ensure ligand binding to the receptor. To optimize binding efficiency, sequential changes can be made to lower the overall free energy while maintaining the secondary structure. Other changes may include the use of stapled residues to maintain the secondary structure.

Peptides can then be anchored for delivery to the cell, tissue, or organ of interest. Anchors may include any click chemistry centered around maleimide or azide-alkyne conjugation where a delivery molecule (e.g., PEG-2000, a lipid, a lipid-PEG conjugate, or a nanoparticle component) can be attached. Similarly, to ensure peptide stability, certain residues may be changed to unnatural amino acids (e.g., Aib or 2-aminoisobutyric acid). Once designed, minor changes could be made to the foundational peptide to either enhance stability or accessibility to conjugation, and then the foundational peptide can be submitted for molecular dynamic (“MD”) simulation. Results of the MD simulation may determine which peptides are synthesized for experimental confirmation.

### Processing System

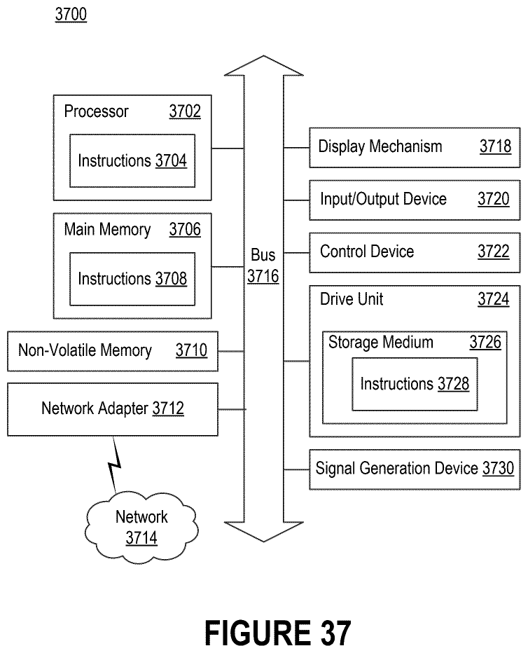

FIG. 37 includes a block diagram of a processing system 3700 in which at least some operations described herein can be implemented. For example, components of the processing system 3700 may be hosted on a computing device that includes a development platform (e.g., development platform 210 of FIG. 2). As noted above, the development platform could alternatively be hosted on a quantum computer, in which case the underlying architecture may differ (e.g., a QPU rather than a processor 3702, quantum memory rather than main memory 3706 or non-volatile memory 3710).

The processing system 3700 can include a processor 3702, main memory 3706, non-volatile memory 3710, network adapter 3712, video display 3718, input/output devices 3720, control device 3722 (e.g., a keyboard or pointing device such as a computer mouse or trackpad), drive unit 3724 including a storage medium 3726, and signal generation device 3730 that are communicatively connected to a bus 3716. The bus 3716 is illustrated as an abstraction that represents one or more physical buses or point-to-point connections that are connected by appropriate bridges, adapters, or controllers. The bus 3716, therefore, can include a system bus, a Peripheral Component Interconnect (“PCI”) bus or PCI-Express bus, a HyperTransport (“HT”) bus, an Industry Standard Architecture (“ISA”) bus, a Small Computer System Interface (“SCSI”) bus, a Universal Serial Bus (“USB”) data interface, an Inter-Integrated Circuit (“I²C”) bus, or a high-performance serial bus developed in accordance with IEEE 1394.

While the main memory 3706, non-volatile memory 3710, and storage medium 3726 are shown to be a single medium, the terms “machine-readable medium” and “storage medium” should be taken to include a single medium or multiple media (e.g., a centralized/distributed database and/or associated caches and servers) that store one or more sets of instructions 3728. The terms “machine-readable medium” and “storage medium” shall also be taken to include any medium that is capable of storing, encoding, or carrying a set of instructions for execution by the processing system 3700.

In general, the routines executed to implement the embodiments of the disclosure can be implemented as part of an operating system or a specific application, component, program, object, module, or sequence of instructions (collectively referred to as “computer programs”). The computer programs typically comprise one or more instructions (e.g., instructions 3704, 3708, 3728) set at various times in various memory and storage devices in a computing device. When read and executed by the processor 3702, the instruction(s) cause the processing system 3700 to perform operations to execute elements involving the various aspects of the present disclosure.

Further examples of machine- and computer-readable media include recordable-type media, such as volatile memory devices and non-volatile memory devices 3710, removable disks, hard disk drives, and optical disks (e.g., Compact Disk Read-Only Memory (“CD-ROMs”) and Digital Versatile Disks (“DVDs”)), and transmission-type media, such as digital and analog communication links.

The network adapter 3712 enables the processing system 3700 to mediate data in a network 3714 with an entity that is external to the processing system 3700 through any communication protocol supported by the processing system 3700 and the external entity. The network adapter 3712 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### Embodiments of Compounds Having Therapeutic Applications Generated by the Present Technology

Compounds having therapeutic applications generated by the present technology include, but are not limited to, peptide ligands of certain receptors. Such compounds are not naturally occurring and are rather designed or otherwise generated by one or more aspects of the present technology. Such peptide ligands may be designed to allosterically and/or orthosterically bind certain receptors. Non-limiting examples of receptors include CD117, cKit, and CD34. The “peptides” and peptoids described herein can be (a) naturally-occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides or recombinant proteins.

The term “peptide” as used herein includes any structure comprised of two or more amino acids, including chemical modifications and derivatives of amino acids. The amino acids forming all or a part of a peptide may be naturally occurring amino acids, stereoisomers and modifications of such amino acids, non-protein amino acids, post-translationally modified amino acids, enzymatically modified amino acids, constructs or structures designed to mimic amino acids, peptoids, and the like, so that the term “peptide” includes pseudopeptides and peptidomimetics, including structures which have a non-peptidic backbone. The term “peptide” also includes dimers or multimers of peptides. A “manufactured” peptide includes a peptide produced by chemical synthesis, recombinant DNA technology, biochemical, or enzymatic fragmentation of larger molecules, combinations of the foregoing or, in general, made by any other method. The term “peptide” includes peptides containing a variable number of amino acid residues, optionally with non-amino acid residue groups at the N- and C-termini, such groups including acyl, acetyl, alkenyl, alkyl, N-alkyl, amine, DBCO, or amide groups, among others.

By employing chemical synthesis, a useful means of production, it is possible to introduce various amino acids which do not naturally occur along the chain, modify the N- or C-terminus, and the like, thereby providing for improved stability and formulation, resistance to protease degradation, and the like. Non-limiting examples of chemical synthesis include solid-phase and solution-phase peptide synthesis.

The terms “bind,” “binding,” “complex,” and “complexing,” refer to all types of physical and chemical binding, reactions, complexing, attraction, chelating and the like.

The present technology includes various rationales when selecting an amino acid residue at one or more positions in the peptide ligand, one or more of which may be accounted for when designing such compounds. Rationales for features of the peptide ligand include increase or decrease Gibbs free energy, increase or decrease a Van der Waals effect, additions of one or more linkages, improving solubility, zwitterionic effect with a conjugate, positive to negative amino acid residue ratios between 4/2 and 6/2, non charged polar residue compositions of less than about 20%, aliphatic hydrophobic residues from about 40% to about 50%, aromatic hydrophobic residues and tertiary structures such as beta sheets, location of amino acid residues to promote or inhibit pairing, serum protein corona repulsive behavior, and specific turn character.

“Amino acids” are molecules containing an amine group, a carboxylic acid group, and a side-chain that is specific to each amino acid. The key elements of an amino acid are carbon, hydrogen, oxygen, and nitrogen and have the generic formula H₂N—CHR—COOH, wherein R represents a side chain group. The various α-amino acids differ in the side-chain moiety that is attached to the α-carbon. The “amino acids” of the present technology include the known naturally occurring protein amino acids, which are referred to by both their common three letter abbreviation and single letter abbreviation. See generally Synthetic Peptides: A User's Guide, G. A. Grant, editor, W.H. Freeman & Co., New York (1992), the teachings of which are incorporated herein by reference, including the text and table set forth at pages 11 through 24. As set forth above, the term “amino acid” also includes stereoisomers and modifications of naturally occurring protein amino acids, non-protein amino acids, post-translationally modified amino acids, enzymatically synthesized amino acids, derivatized amino acids, constructs or structures designed to mimic amino acids, peptoids, and the like. Modified and unusual amino acids are described generally in Synthetic Peptides: A User's Guide, supra; Hruby et al., Biochem. J. 268:249-262 (1990); and Toniolo, Int. J. Peptide Protein Res. 35:287-300 (1990); the teachings of all of which are incorporated herein by reference.

The phrase “amino acid side chain moiety” used herein, including as used in the specification and claims, includes any side chain of any amino acid, as the term “amino acid” is defined herein. This thus includes the side chain moiety present in naturally occurring amino acids. It further includes side chain moieties in modified naturally occurring amino acids, such as glycosylated amino acids. It further includes side chain moieties in stereoisomers and modifications of naturally occurring protein amino acids, non-protein amino acids, post-translationally modified amino acids, enzymatically synthesized amino acids, derivatized amino acids, constructs, or structures designed to mimic amino acids, and the like. For example, the side chain moiety of any amino acid disclosed herein is included within the definition. A “derivative” of an amino acid side chain moiety is included within the definition of an amino acid side chain moiety.

The “derivative” of an amino acid side chain moiety includes any modification to or variation in any amino acid side chain moieties, including a modification of naturally occurring amino acid side chain moieties. By way of example, derivatives of amino acid side chain moieties include straight chain or branched, cyclic or noncyclic, substituted or unsubstituted, saturated or unsaturated, alkyl, aryl or aralkyl moieties as well as small molecule ligand conjugates.

In the peptides described herein, conventional amino acid residues have their conventional meaning as given in Chapter 2400, of the Manual of Patent Examining Procedure, 8^{th} Ed. Thus, “Ala” is alanine; “Arg” is arginine; “Asn” is asparagine; “Asp” is aspartic acid; “Cys” is cysteine; “Gln” is glutamine; “Glu” is glutamic acid; “His” is histidine; “Ile” is isoleucine; “Leu” is leucine; “Lys” is lysine; “Met” is methionine; “Phe” is phenylalanine; “Pro” is proline; “Ser” is serine; Thr is threonine; “Trp” is tryptophan; “Tyr” is tryosine; and “Val” is valine. Unless otherwise indicated, all amino acids abbreviations represent either isomer, i.e., the L-isomer, the D-isomer, or combinations thereof can be used. Non-standard amino acids are “Nle” is norleucine and so on.

An alpha (α)-amino acid has the generic formula H₂N—C_{α}HR—COOH, where R is a side chain moiety and the amino group is attached to the carbon atom immediately adjacent to the carboxylate group (i.e., the α-carbon). Other types of amino acids exist when the amino group is attached to a different carbon atom. For example, beta (β)-amino acids, the carbon atom to which the amino group is attached is separated from the carboxylate group by one carbon atom, C_{β}. For example, α-alanine has the formula H₂N—C_{α}H(CH₃)—COOH. In contrast, β-alanine has the general formula H₂N—C_{β}H₂—C_{α}H₂—COOH (i.e., 3-aminopropanoic acid)

When β-amino acids are incorporated into peptides, two main types of β-peptides exist: those with the side chain residue, R, on the carbon next to the amine are called β³ peptides and those with the side chain residue on the carbon next to the carbonyl group are called β² amino acids. As a non-limiting example, “β-valine” can refer to:
—NH—C_{β}H₂—C_{α}H(CH₃)₂—CO—, i.e., β²-valine (R on carboxy side);
—NH—C_{β}H(CH₃)₂—C_{α}H₂—CO—, i.e., β³-valine (R on amino side); or
—NH—C_{β}H(CH₃)₂—C_{α}H(CH₃)₂—CO—, i.e., β^{2,3}-valine (R at both positions).

Gamma (γ)-amino acids are amino acids with the carbon atom to which the amino group attaches is separated from the carboxylate moiety by two carbon atoms. For example, γ-amino butyric acid has the formula, H₂N—C_{γ}H₂—C_{β}H₂—C_{α}H₂—COOH.

For additional modified and unusual amino acids, see § 2422 of the MPEP, particularly Table 4 at 2400-24. Additionally, “Ac” indicates N-acetyl and “NH₂” indicates an amine group, typically added on the C-terminus of a polypeptide. Accordingly, as used herein, an —NH₂ moiety on the C-terminus of a peptide indicates an amidated C-terminus.

A peptide or aliphatic moiety is “acylated” when an alkyl or substituted alkyl group as defined above is bonded through one or more carbonyl {—(C═O)—} groups. A peptide is most usually acylated at the N-terminus.

An “amine” includes compounds that contain an amine group (—NH₂).

An “amide” includes compounds that have a trivalent nitrogen attached to a carbonyl group (i.e., —CO—NH₂), such as for example methylamide, ethylamide, propylamide, and the like. A peptide is most usually amidated at the C-terminus by the addition of an amine (—NH₂) moiety to the C-terminal carboxyl group.

Amino acids, including stereoisomers and modifications of naturally occurring amino acids, protein amino acids, non-protein amino acids, post-translationally modified amino acids, enzymatically synthesized amino acids, derivatized amino acids, constructs, or structures designed to mimic amino acids (peptide mimetics), and the like, including all of the foregoing, are sometimes referred to herein as “residues.”

A peptide or amino acid “mimetic” is a non-amino acid molecule that mimics a peptide (a chain of amino acids) or one amino acid residue.

In some embodiments, variants of the peptide ligands of the present technology may be used. “Variants” include protein sequences having one or more amino acid additions, deletions, stop positions, or substitutions, as compared to a peptide sequence disclosed elsewhere herein.

An amino acid substitution may be a conservative or a non-conservative substitution. Variants of the peptide ligands of the present technology include those having one or more conservative amino acid substitutions. A “conservative substitution” or “conservative amino acid substitution” involves a substitution found in one of the following conservative substitutions groups: Group 1: Ala, Gly, Ser, Thr; Group 2: Glu, Asp; Group 3: Asn, Glu; Group 4: R, K, H; Group 5: Ile, Leu, Met, Val; and Group 6: Phe, Tyr, Trp.

Additionally, amino acids may be grouped into conservative substitution groups by similar function, chemical structure, or composition (e.g., hydrophobic with non-polar side chain, hydrophilic with polar side chain, acidic, basic, aliphatic, aromatic, positively charged, negatively charged, containing a side group such as a conjugation group, a small molecule ligand group, a cross-linking group, or a conjugation site for another molecule on its side group, or sulfur-containing). For example, an aliphatic grouping may include, for purposes of substitution, Gly, Ala, Val, Leu, and Ile. Other groups including amino acids that are considered conservative substitutions for one another include: sulfur-containing: Met and Cys; acidic: Asp, Glu, Asn, Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp.

Non-conservative substitutions include those that significantly affect: the structure of the peptide backbone in the area of the alteration (e.g., the alpha-helical or beta-sheet structure); the charge or hydrophobicity of the molecule at the target site; or the bulk of the side chain. Non-conservative substitutions which in general are expected to produce the greatest changes in the protein's properties are those in which (i) a hydrophilic residue (e.g. Ser or Thr) may be substituted for (or by) a hydrophobic residue (e.g. Leu, Ile, Phe, Val, or Ala); (ii) a Cys or Phe may be substituted for (or by) any other residue; (iii) a residue having an electropositive side chain (e.g. Lys, Arg, or His) may be substituted for (or by) an electronegative residue (e.g. Gln or Asp); or (iv) a residue having a bulky side chain (e.g. Phe), may be substituted for (or by) one not having a bulky side chain, (e.g. Gly). Additional information is found in Creighton (1984) Proteins, W.H. Freeman and Company.

In some embodiments, the present technology provides non-naturally occurring peptide ligands designed or otherwise generated according to one or more of the rationales described herein when binding to the target receptor is the desired outcome of the designed peptide ligand. Non-limiting example peptide ligands of CD34 designed in accordance with the present technology are recited in Table 1 except for peptides corresponding to SEQ ID NOs: 86 and 87, which correspond to CD34 and an exemplary PISA peptide designed in accordance with the present technology.

TABLE 1
Sequences of Exemplary CD34 Peptide Ligands and CD34

| SEQ | | |
|---|---|---|
| ID NO | Peptide Sequence | Peptide Identity |
| 1 | RAYNTSTGLALCYAS | Non-naturally occurring CD34 peptide ligand 1 |
| 2 | NAYNTSTGLALCYAS | Non-naturally occurring CD34 peptide ligand 2 |
| 3 | RVYNTSTGLALCYAS | Non-naturally occurring CD34 peptide ligand 3 |
| 4 | NVYNTSTGLALCYAS | Non-naturally occurring CD34 peptide ligand 4 |
| 5 | RAYNTSTGLALCYAN | Non-naturally occurring CD34 peptide ligand 5 |
| 6 | NAYNTSTGLALCYAN | Non-naturally occurring CD34 peptide ligand 6 |
| 7 | RVYNTSTGLALCYAN | Non-naturally occurring CD34 peptide ligand 7 |
| 8 | NVYNTSTGLALCYAN | Non-naturally occurring CD34 peptide ligand 8 |
| 9 | RAYNTSTGGLALCYAS | Non-naturally occurring CD34 peptide ligand 9 |
| 10 | NAYNTSTGGLALCYAS | Non-naturally occurring CD34 peptide ligand 10 |
| 11 | RVYNTSTGGLALCYAS | Non-naturally occurring CD34 peptide ligand 11 |
| 12 | NVYNTSTGGLALCYAS | Non-naturally occurring CD34 peptide ligand 12 |
| 13 | RAYNTSTGGLALCYAN | Non-naturally occurring CD34 peptide ligand 13 |
| 14 | NAYNTSTGGLALCYAN | Non-naturally occurring CD34 peptide ligand 14 |
| 15 | RVYNTSTGGLALCYAN | Non-naturally occurring CD34 peptide ligand 15 |
| 16 | NVYNTSTGGLALCYAN | Non-naturally occurring CD34 peptide ligand 16 |
| 17 | RAYNTSTCGLALCYAN | Non-naturally occurring CD34 peptide ligand 17 |
| 18 | NAYNTSTCGLALCYAN | Non-naturally occurring CD34 peptide ligand 18 |
| 19 | RVYNTSTCGLALCYAN | Non-naturally occurring CD34 peptide ligand 19 |
| 20 | NVYNTSTCGLALCYAN | Non-naturally occurring CD34 peptide ligand 20 |
| 21 | RAYNTSTCGGLALCYAN | Non-naturally occurring CD34 peptide ligand 21 |
| 22 | NAYNTSTCGGLALCYAN | Non-naturally occurring CD34 peptide ligand 22 |
| 23 | RVYNTSTCGGLALCYAN | Non-naturally occurring CD34 peptide ligand 23 |
| 24 | NVYNTSTCGGLALCYAN | Non-naturally occurring CD34 peptide ligand 24 |
| 25 | RAYNTSTCGLALCYAS | Non-naturally occurring CD34 peptide ligand 25 |
| 26 | NAYNTSTCGLALCYAS | Non-naturally occurring CD34 peptide ligand 26 |
| 27 | RVYNTSTCGLALCYAS | Non-naturally occurring CD34 peptide ligand 27 |
| 28 | NVYNTSTCGLALCYAS | Non-naturally occurring CD34 peptide ligand 28 |
| 29 | RAYNTSTCGGLALCYAS | Non-naturally occurring CD34 peptide ligand 29 |
| 30 | NAYNTSTCGGLALCYAS | Non-naturally occurring CD34 peptide ligand 30 |
| 31 | RVYNTSTCGGLALCYAS | Non-naturally occurring CD34 peptide ligand 31 |
| 32 | NVYNTSTCGGLALCYAS | Non-naturally occurring CD34 peptide ligand 32 |
| 33 | RAYNTSTAibLALCYAN | Non-naturally occurring CD34 peptide ligand 33 |
| 34 | NAYNTSTAibLALCYAN | Non-naturally occurring CD34 peptide ligand 34 |
| 35 | RVYNTSTAibLALCYAN | Non-naturally occurring CD34 peptide ligand 35 |
| 36 | NVYNTSTAibLALCYAN | Non-naturally occurring CD34 peptide ligand 36 |
| 37 | RAYNTSCAibLALCYAN | Non-naturally occurring CD34 peptide ligand 37 |
| 38 | NAYNTSCAibLALCYAN | Non-naturally occurring CD34 peptide ligand 38 |
| 39 | RVYNTSCAibLALCYAN | Non-naturally occurring CD34 peptide ligand 39 |
| 40 | NVYNTSCAibLALCYAN | Non-naturally occurring CD34 peptide ligand 40 |
| 41 | RAYNTSTAibLALCYAS | Non-naturally occurring CD34 peptide ligand 41 |
| 42 | NAYNTSTAibLALCYAS | Non-naturally occurring CD34 peptide ligand 42 |
| 43 | RVYNTSTAibLALCYAS | Non-naturally occurring CD34 peptide ligand 43 |
| 44 | NVYNTSTAibLALCYAS | Non-naturally occurring CD34 peptide ligand 44 |
| 45 | RAYNTSCAibLALCYAS | Non-naturally occurring CD34 peptide ligand 45 |
| 46 | NAYNTSCAibLALCYAS | Non-naturally occurring CD34 peptide ligand 46 |
| 47 | RVYNTSCAibLALCYAS | Non-naturally occurring CD34 peptide ligand 47 |
| 48 | NVYNTSCAibLALCYAS | Non-naturally occurring CD34 peptide ligand 48 |
| 49 | RAYNTSTSTAibAibLALCYAN | Non-naturally occurring CD34 peptide ligand 49 |
| 50 | NAYNTSTSTAibAibLALCYAN | Non-naturally occurring CD34 peptide ligand 50 |
| 51 | RVYNTSTSTAibAibLALCYAN | Non-naturally occurring CD34 peptide ligand 51 |
| 52 | NVYNTSTSTAibAibLALCYAN | Non-naturally occurring CD34 peptide ligand 52 |
| 53 | RAYNTSCAibAibLALCYAN | Non-naturally occurring CD34 peptide ligand 53 |
| 54 | NAYNTSCAibAibLALCYAN | Non-naturally occurring CD34 peptide ligand 54 |
| 55 | RVYNTSCAibAibLALCYAN | Non-naturally occurring CD34 peptide ligand 55 |
| 56 | NVYNTSCAibAibLALCYAN | Non-naturally occurring CD34 peptide ligand 56 |
| 57 | RAYNTSTSTAibAibLALCYAS | Non-naturally occurring CD34 peptide ligand 57 |
| 58 | NAYNTSTSTAibAibLALCYAS | Non-naturally occurring CD34 peptide ligand 58 |
| 59 | RVYNTSTSTAibAibLALCYAS | Non-naturally occurring CD34 peptide ligand 59 |
| 60 | NVYNTSTSTAibAibLALCYAS | Non-naturally occurring CD34 peptide ligand 60 |
| 61 | RAYNTSCAibAibLALCYAS | Non-naturally occurring CD34 peptide ligand 61 |
| 62 | NAYNTSCAibAibLALCYAS | Non-naturally occurring CD34 peptide ligand 62 |
| 63 | RVYNTSCAibAibLALCYAS | Non-naturally occurring CD34 peptide ligand 63 |
| 64 | NVYNTSCAibAibLALCYAS | Non-naturally occurring CD34 peptide ligand 64 |
| 65 | RAYNTSTGGLALEYAS | Non-naturally occurring CD34 peptide ligand 65 |
| 66 | RAYNTSTGGEELEYAS | Non-naturally occurring CD34 peptide ligand 66 |
| 67 | RAYNTSTGSGEELEYAS | Non-naturally occurring CD34 peptide ligand 67 |
| 68 | RAYNTSTG(ϵ-azido- | Non-naturally occurring CD34 peptide ligand 68 |
| | Nle)GLALEYAS | |
| 69 | RAYNTSTG(ϵ-azido- | Non-naturally occurring CD34 peptide ligand 69 |
| | Nle)GEELEYAS | |
| 70 | RAYNTSTGS(ϵ-azido- | Non-naturally occurring CD34 peptide ligand 70 |
| | Nle)GEELEYAS | |
| 71 | RAYNESTGGEELEYAS | Non-naturally occurring CD34 peptide ligand 71 |
| 72 | RAYNESTGSGEELEYAS | Non-naturally occurring CD34 peptide ligand 72 |
| 73 | RAYNESTGSGSGEELEYAS | Non-naturally occurring CD34 peptide ligand 73 |
| 74 | RAYNESTG(ϵ-azido- | Non-naturally occurring CD34 peptide ligand 74 |
| | Nle)GEELEYAS | |
| 75 | RAYNESTGS(ϵ-azido- | Non-naturally occurring CD34 peptide ligand 75 |
| | Nle)GEELEYAS | |
| 76 | RAYNESTGS(ϵ-azido- | Non-naturally occurring CD34 peptide ligand 76 |
| | Nle)GSGEELEYAS | |
| 77 | RAYNRSTGGRRLRYAS | Non-naturally occurring CD34 peptide ligand 77 |
| 78 | RAYNRSTGSGRRLRYAS | Non-naturally occurring CD34 peptide ligand 78 |
| 79 | RAYNRSTGSGSGESLRYAS | Non-naturally occurring CD34 peptide ligand 79 |
| 80 | RAYNRSTG(ϵ-azido- | Non-naturally occurring CD34 peptide ligand 80 |
| | Nle)GRRLRYAS | |
| 81 | RAYNRSTGS(ϵ-azido- | Non-naturally occurring CD34 peptide ligand 81 |
| | Nle)GRRLCRAS | |
| 82 | RAYNRSTGS(ϵ-azido- | Non-naturally occurring CD34 peptide ligand 82 |
| | Nle)GSGESLRYAS | |
| 83 | RAYNRSTGS(ϵ-azido- | Non-naturally occurring CD34 peptide ligand 83 |
| | Nle)GSGRRLRYAS | |
| 84 | RAYNESTGS(ϵ-azido- | Non-naturally occurring CD34 peptide ligand 84 |
| | Nle)GSGESLEYAS | |
| 85 | RAYNRSTGS(ϵ-azido- | Non-naturally occurring CD34 peptide ligand 85 |
| | Nle)GSGRSLRYAS | |
| 86 | MIASQFLSAL TLVLLIKESG | CD34 |
| | AWSYNTSTEA | |
| | MTYDEASAYC QQRYTHLVAI | |
| | QNKEEIEYLN SILSYSPSYY | |
| | WIGIRKVNNV | |
| | WVWVGTQKPL | |
| | TEEAKNWAPG | |
| | EPNNRQKDED CVEIYIKREK | |
| | DVGMWNDERC | |
| | SKKKLALCYT A | |
| 87 | WSYNTSTLALCYTA | PISA peptide |

In some embodiments, the non-naturally occurring CD34 peptide ligand comprises an amino acid sequence at least about 80%, 85%, 90%, 95%, 99%, or 100% identical to a sequence selected from the group consisting of SEQ ID NO: 1-85. In some embodiments, the non-naturally occurring CD34 peptide ligand comprises an amino acid sequence selected from the group consisting of SEQ ID NO: 1-85.

In some embodiments, the non-naturally occurring CD34 peptide ligand comprises an amino acid sequence at least about 80%, 85%, 90%, 95%, 99%, or 100% identical to SEQ ID NO: 1. In some embodiments, the non-naturally occurring CD34 peptide ligand comprises SEQ ID NO: 1. In some embodiments, the non-naturally occurring CD34 peptide ligand comprises an amino acid sequence at least about 80%, 85%, 90%, 95%, 99%, or 100% identical to SEQ ID NO: 2. In some embodiments, the non-naturally occurring CD34 peptide ligand comprises SEQ ID NO: 2. In some embodiments, the non-naturally occurring CD34 peptide ligand comprises an amino acid sequence at least about 80%, 85%, 90%, 95%, 99%, or 100% identical to SEQ ID NO: 3. In some embodiments, the non-naturally occurring CD34 peptide ligand comprises SEQ ID NO: 3. In some embodiments, the non-naturally occurring CD34 peptide ligand comprises an amino acid sequence at least about 80%, 85%, 90%, 95%, 99%, or 100% identical to SEQ ID NO: 4. In some embodiments, the non-naturally occurring CD34 peptide ligand comprises SEQ ID NO: 4. In some embodiments, the non-naturally occurring CD34 peptide ligand comprises an amino acid sequence at least about 80%, 85%, 90%, 95%, 99%, or 100% identical to SEQ ID NO: 5. In some embodiments, the non-naturally occurring CD34 peptide ligand comprises SEQ ID NO: 5. In some embodiments, the non-naturally occurring CD34 peptide ligand comprises an amino acid sequence at least about 80%, 85%, 90%, 95%, 99%, or 100% identical to SEQ ID NO: 6. In some embodiments, the non-naturally occurring CD34 peptide ligand comprises SEQ ID NO: 6. In some embodiments, the non-naturally occurring CD34 peptide ligand comprises an amino acid sequence at least about 80%, 85%, 90%, 95%, 99%, or 100% identical to SEQ ID NO: 7. In some embodiments, the non-naturally occurring CD34 peptide ligand comprises SEQ ID NO: 7. In some embodiments, the non-naturally occurring CD34 peptide ligand comprises an amino acid sequence at least about 80%, 85%, 90%, 95%, 99%, or 100% identical to SEQ ID NO: 8. In some embodiments, the non-naturally occurring CD34 peptide ligand comprises SEQ ID NO: 8. In some embodiments, the non-naturally occurring CD34 peptide ligand comprises an amino acid sequence at least about 80%, 85%, 90%, 95%, 99%, or 100% identical to SEQ ID NO: 9. In some embodiments, the non-naturally occurring CD34 peptide ligand comprises SEQ ID NO: 9. In some embodiments, the non-naturally occurring CD34 peptide ligand comprises an amino acid sequence at least about 80%, 85%, 90%, 95%, 99%, or 100% identical to SEQ ID NO: 10. In some embodiments, the non-naturally occurring CD34 peptide ligand comprises SEQ ID NO: 10. In some embodiments, the non-naturally occurring CD34 peptide ligand comprises an amino acid sequence at least about 80%, 85%, 90%, 95%, 99%, or 100% identical to SEQ ID NO: 11. In some embodiments, the non-naturally occurring CD34 peptide ligand comprises SEQ ID NO: 11. In some embodiments, the non-naturally occurring CD34 peptide ligand comprises an amino acid sequence at least about 80%, 85%, 90%, 95%, 99%, or 100% identical to SEQ ID NO: 12. In some embodiments, the non-naturally occurring CD34 peptide ligand comprises SEQ ID NO: 12. In some embodiments, the non-naturally occurring CD34 peptide ligand comprises an amino acid sequence at least about 80%, 85%, 90%, 95%, 99%, or 100% identical to SEQ ID NO: 13. In some embodiments, the non-naturally occurring CD34 peptide ligand comprises SEQ ID NO: 13. In some embodiments, the non-naturally occurring CD34 peptide ligand comprises an amino acid sequence at least about 80%, 85%, 90%, 95%, 99%, or 100% identical to SEQ ID NO: 14. In some embodiments, the non-naturally occurring CD34 peptide ligand comprises SEQ ID NO: 14. In some embodiments, the non-naturally occurring CD34 peptide ligand comprises an amino acid sequence at least about 80%, 85%, 90%, 95%, 99%, or 100% identical to SEQ ID NO: 15. In some embodiments, the non-naturally occurring CD34 peptide ligand comprises SEQ ID NO: 15. In some embodiments, the non-naturally occurring CD34 peptide ligand comprises an amino acid sequence at least about 80%, 85%, 90%, 95%, 99%, or 100% identical to SEQ ID NO: 16. In some embodiments, the non-naturally occurring CD34 peptide ligand comprises SEQ ID NO: 16. In some embodiments, the non-naturally occurring CD34 peptide ligand comprises an amino acid sequence at least about 80%, 85%, 90%, 95%, 99%, or 100% identical to SEQ ID NO: 17. In some embodiments, the non-naturally occurring CD34 peptide ligand comprises SEQ ID NO: 17. In some embodiments, the non-naturally occurring CD34 peptide ligand comprises an amino acid sequence at least about 80%, 85%, 90%, 95%, 99%, or 100% identical to SEQ ID NO: 18. In some embodiments, the non-naturally occurring CD34 peptide ligand comprises SEQ ID NO: 18. In some embodiments, the non-naturally occurring CD34 peptide ligand comprises an amino acid sequence at least about 80%, 85%, 90%, 95%, 99%, or 100% identical to SEQ ID NO: 19. In some embodiments, the non-naturally occurring CD34 peptide ligand comprises SEQ ID NO: 19. In some embodiments, the non-naturally occurring CD34 peptide ligand comprises an amino acid sequence at least about 80%, 85%, 90%, 95%, 99%, or 100% identical to SEQ ID NO: 20. In some embodiments, the non-naturally occurring CD34 peptide ligand comprises SEQ ID NO: 20. In some embodiments, the non-naturally occurring CD34 peptide ligand comprises an amino acid sequence at least about 80%, 85%, 90%, 95%, 99%, or 100% identical to SEQ ID NO: 21. In some embodiments, the non-naturally occurring CD34 peptide ligand comprises SEQ ID NO: 21. In some embodiments, the non-naturally occurring CD34 peptide ligand comprises an amino acid sequence at least about 80%, 85%, 90%, 95%, 99%, or 100% identical to SEQ ID NO: 22. In some embodiments, the non-naturally occurring CD34 peptide ligand comprises SEQ ID NO: 22. In some embodiments, the non-naturally occurring CD34 peptide ligand comprises an amino acid sequence at least about 80%, 85%, 90%, 95%, 99%, or 100% identical to SEQ ID NO: 23. In some embodiments, the non-naturally occurring CD34 peptide ligand comprises SEQ ID NO: 23. In some embodiments, the non-naturally occurring CD34 peptide ligand comprises an amino acid sequence at least about 80%, 85%, 90%, 95%, 99%, or 100% identical to SEQ ID NO: 24. In some embodiments, the non-naturally occurring CD34 peptide ligand comprises SEQ ID NO: 24. In some embodiments, the non-naturally occurring CD34 peptide ligand comprises an amino acid sequence at least about 80%, 85%, 90%, 95%, 99%, or 100% identical to SEQ ID NO: 25. In some embodiments, the non-naturally occurring CD34 peptide ligand comprises SEQ ID NO: 25. In some embodiments, the non-naturally occurring CD34 peptide ligand comprises an amino acid sequence at least about 80%, 85%, 90%, 95%, 99%, or 100% identical to SEQ ID NO: 26. In some embodiments, the non-naturally occurring CD34 peptide ligand comprises SEQ ID NO: 26. In some embodiments, the non-naturally occurring CD34 peptide ligand comprises an amino acid sequence at least about 80%, 85%, 90%, 95%, 99%, or 100% identical to SEQ ID NO: 27. In some embodiments, the non-naturally occurring CD34 peptide ligand comprises SEQ ID NO: 27. In some embodiments, the non-naturally occurring CD34 peptide ligand comprises an amino acid sequence at least about 80%, 85%, 90%, 95%, 99%, or 100% identical to SEQ ID NO: 28. In some embodiments, the non-naturally occurring CD34 peptide ligand comprises SEQ ID NO: 28. In some embodiments, the non-naturally occurring CD34 peptide ligand comprises an amino acid sequence at least about 80%, 85%, 90%, 95%, 99%, or 100% identical to SEQ ID NO: 29. In some embodiments, the non-naturally occurring CD34 peptide ligand comprises SEQ ID NO: 29. In some embodiments, the non-naturally occurring CD34 peptide ligand comprises an amino acid sequence at least about 80%, 85%, 90%, 95%, 99%, or 100% identical to SEQ ID NO: 30. In some embodiments, the non-naturally occurring CD34 peptide ligand comprises SEQ ID NO: 30. In some embodiments, the non-naturally occurring CD34 peptide ligand comprises an amino acid sequence at least about 80%, 85%, 90%, 95%, 99%, or 100% identical to SEQ ID NO: 31. In some embodiments, the non-naturally occurring CD34 peptide ligand comprises SEQ ID NO: 31. In some embodiments, the non-naturally occurring CD34 peptide ligand comprises an amino acid sequence at least about 80%, 85%, 90%, 95%, 99%, or 100% identical to SEQ ID NO: 32. In some embodiments, the non-naturally occurring CD34 peptide ligand comprises SEQ ID NO: 32. In some embodiments, the non-naturally occurring CD34 peptide ligand comprises an amino acid sequence at least about 80%, 85%, 90%, 95%, 99%, or 100% identical to SEQ ID NO: 33. In some embodiments, the non-naturally occurring CD34 peptide ligand comprises SEQ ID NO: 33. In some embodiments, the non-naturally occurring CD34 peptide ligand comprises an amino acid sequence at least about 80%, 85%, 90%, 95%, 99%, or 100% identical to SEQ ID NO: 34. In some embodiments, the non-naturally occurring CD34 peptide ligand comprises SEQ ID NO: 34. In some embodiments, the non-naturally occurring CD34 peptide ligand comprises an amino acid sequence at least about 80%, 85%, 90%, 95%, 99%, or 100% identical to SEQ ID NO: 35. In some embodiments, the non-naturally occurring CD34 peptide ligand comprises SEQ ID NO: 35. In some embodiments, the non-naturally occurring CD34 peptide ligand comprises an amino acid sequence at least about 80%, 85%, 90%, 95%, 99%, or 100% identical to SEQ ID NO: 36. In some embodiments, the non-naturally occurring CD34 peptide ligand comprises SEQ ID NO: 36. In some embodiments, the non-naturally occurring CD34 peptide ligand comprises an amino acid sequence at least about 80%, 85%, 90%, 95%, 99%, or 100% identical to SEQ ID NO: 37. In some embodiments, the non-naturally occurring CD34 peptide ligand comprises SEQ ID NO: 37. In some embodiments, the non-naturally occurring CD34 peptide ligand comprises an amino acid sequence at least about 80%, 85%, 90%, 95%, 99%, or 100% identical to SEQ ID NO: 38. In some embodiments, the non-naturally occurring CD34 peptide ligand comprises SEQ ID NO: 38. In some embodiments, the non-naturally occurring CD34 peptide ligand comprises an amino acid sequence at least about 80%, 85%, 90%, 95%, 99%, or 100% identical to SEQ ID NO: 39. In some embodiments, the non-naturally occurring CD34 peptide ligand comprises SEQ ID NO: 39. In some embodiments, the non-naturally occurring CD34 peptide ligand comprises an amino acid sequence at least about 80%, 85%, 90%, 95%, 99%, or 100% identical to SEQ ID NO: 40. In some embodiments, the non-naturally occurring CD34 peptide ligand comprises SEQ ID NO: 40. In some embodiments, the non-naturally occurring CD34 peptide ligand comprises an amino acid sequence at least about 80%, 85%, 90%, 95%, 99%, or 100% identical to SEQ ID NO: 41. In some embodiments, the non-naturally occurring CD34 peptide ligand comprises SEQ ID NO: 41. In some embodiments, the non-naturally occurring CD34 peptide ligand comprises an amino acid sequence at least about 80%, 85%, 90%, 95%, 99%, or 100% identical to SEQ ID NO: 42. In some embodiments, the non-naturally occurring CD34 peptide ligand comprises SEQ ID NO: 42. In some embodiments, the non-naturally occurring CD34 peptide ligand comprises an amino acid sequence at least about 80%, 85%, 90%, 95%, 99%, or 100% identical to SEQ ID NO: 43. In some embodiments, the non-naturally occurring CD34 peptide ligand comprises SEQ ID NO: 43. In some embodiments, the non-naturally occurring CD34 peptide ligand comprises an amino acid sequence at least about 80%, 85%, 90%, 95%, 99%, or 100% identical to SEQ ID NO: 44. In some embodiments, the non-naturally occurring CD34 peptide ligand comprises SEQ ID NO: 44. In some embodiments, the non-naturally occurring CD34 peptide ligand comprises an amino acid sequence at least about 80%, 85%, 90%, 95%, 99%, or 100% identical to SEQ ID NO: 45. In some embodiments, the non-naturally occurring CD34 peptide ligand comprises SEQ ID NO: 45. In some embodiments, the non-naturally occurring CD34 peptide ligand comprises an amino acid sequence at least about 80%, 85%, 90%, 95%, 99%, or 100% identical to SEQ ID NO: 46. In some embodiments, the non-naturally occurring CD34 peptide ligand comprises SEQ ID NO: 46. In some embodiments, the non-naturally occurring CD34 peptide ligand comprises an amino acid sequence at least about 80%, 85%, 90%, 95%, 99%, or 100% identical to SEQ ID NO: 47. In some embodiments, the non-naturally occurring CD34 peptide ligand comprises SEQ ID NO: 47. In some embodiments, the non-naturally occurring CD34 peptide ligand comprises an amino acid sequence at least about 80%, 85%, 90%, 95%, 99%, or 100% identical to SEQ ID NO: 48. In some embodiments, the non-naturally occurring CD34 peptide ligand comprises SEQ ID NO: 48. In some embodiments, the non-naturally occurring CD34 peptide ligand comprises an amino acid sequence at least about 80%, 85%, 90%, 95%, 99%, or 100% identical to SEQ ID NO: 49. In some embodiments, the non-naturally occurring CD34 peptide ligand comprises SEQ ID NO: 49. In some embodiments, the non-naturally occurring CD34 peptide ligand comprises an amino acid sequence at least about 80%, 85%, 90%, 95%, 99%, or 100% identical to SEQ ID NO: 50. In some embodiments, the non-naturally occurring CD34 peptide ligand comprises SEQ ID NO: 50. In some embodiments, the non-naturally occurring CD34 peptide ligand comprises an amino acid sequence at least about 80%, 85%, 90%, 95%, 99%, or 100% identical to SEQ ID NO: 51. In some embodiments, the non-naturally occurring CD34 peptide ligand comprises SEQ ID NO: 51. In some embodiments, the non-naturally occurring CD34 peptide ligand comprises an amino acid sequence at least about 80%, 85%, 90%, 95%, 99%, or 100% identical to SEQ ID NO: 52. In some embodiments, the non-naturally occurring CD34 peptide ligand comprises SEQ ID NO: 52. In some embodiments, the non-naturally occurring CD34 peptide ligand comprises an amino acid sequence at least about 80%, 85%, 90%, 95%, 99%, or 100% identical to SEQ ID NO: 53. In some embodiments, the non-naturally occurring CD34 peptide ligand comprises SEQ ID NO: 53. In some embodiments, the non-naturally occurring CD34 peptide ligand comprises an amino acid sequence at least about 80%, 85%, 90%, 95%, 99%, or 100% identical to SEQ ID NO: 54. In some embodiments, the non-naturally occurring CD34 peptide ligand comprises SEQ ID NO: 54. In some embodiments, the non-naturally occurring CD34 peptide ligand comprises an amino acid sequence at least about 80%, 85%, 90%, 95%, 99%, or 100% identical to SEQ ID NO: 55. In some embodiments, the non-naturally occurring CD34 peptide ligand comprises SEQ ID NO: 55. In some embodiments, the non-naturally occurring CD34 peptide ligand comprises an amino acid sequence at least about 80%, 85%, 90%, 95%, 99%, or 100% identical to SEQ ID NO: 56. In some embodiments, the non-naturally occurring CD34 peptide ligand comprises SEQ ID NO: 56. In some embodiments, the non-naturally occurring CD34 peptide ligand comprises an amino acid sequence at least about 80%, 85%, 90%, 95%, 99%, or 100% identical to SEQ ID NO: 57. In some embodiments, the non-naturally occurring CD34 peptide ligand comprises SEQ ID NO: 57. In some embodiments, the non-naturally occurring CD34 peptide ligand comprises an amino acid sequence at least about 80%, 85%, 90%, 95%, 99%, or 100% identical to SEQ ID NO: 58. In some embodiments, the non-naturally occurring CD34 peptide ligand comprises SEQ ID NO: 58. In some embodiments, the non-naturally occurring CD34 peptide ligand comprises an amino acid sequence at least about 80%, 85%, 90%, 95%, 99%, or 100% identical to SEQ ID NO: 59. In some embodiments, the non-naturally occurring CD34 peptide ligand comprises SEQ ID NO: 59. In some embodiments, the non-naturally occurring CD34 peptide ligand comprises an amino acid sequence at least about 80%, 85%, 90%, 95%, 99%, or 100% identical to SEQ ID NO: 60. In some embodiments, the non-naturally occurring CD34 peptide ligand comprises SEQ ID NO: 60. In some embodiments, the non-naturally occurring CD34 peptide ligand comprises an amino acid sequence at least about 80%, 85%, 90%, 95%, 99%, or 100% identical to SEQ ID NO: 61. In some embodiments, the non-naturally occurring CD34 peptide ligand comprises SEQ ID NO: 61. In some embodiments, the non-naturally occurring CD34 peptide ligand comprises an amino acid sequence at least about 80%, 85%, 90%, 95%, 99%, or 100% identical to SEQ ID NO: 62. In some embodiments, the non-naturally occurring CD34 peptide ligand comprises SEQ ID NO: 62. In some embodiments, the non-naturally occurring CD34 peptide ligand comprises an amino acid sequence at least about 80%, 85%, 90%, 95%, 99%, or 100% identical to SEQ ID NO: 63. In some embodiments, the non-naturally occurring CD34 peptide ligand comprises SEQ ID NO: 63. In some embodiments, the non-naturally occurring CD34 peptide ligand comprises an amino acid sequence at least about 80%, 85%, 90%, 95%, 99%, or 100% identical to SEQ ID NO: 64. In some embodiments, the non-naturally occurring CD34 peptide ligand comprises SEQ ID NO: 64. In some embodiments, the non-naturally occurring CD34 peptide ligand comprises an amino acid sequence at least about 80%, 85%, 90%, 95%, 99%, or 100% identical to SEQ ID NO: 65. In some embodiments, the non-naturally occurring CD34 peptide ligand comprises SEQ ID NO: 65. In some embodiments, the non-naturally occurring CD34 peptide ligand comprises an amino acid sequence at least about 80%, 85%, 90%, 95%, 99%, or 100% identical to SEQ ID NO: 66. In some embodiments, the non-naturally occurring CD34 peptide ligand comprises SEQ ID NO: 66. In some embodiments, the non-naturally occurring CD34 peptide ligand comprises an amino acid sequence at least about 80%, 85%, 90%, 95%, 99%, or 100% identical to SEQ ID NO: 67. In some embodiments, the non-naturally occurring CD34 peptide ligand comprises SEQ ID NO: 67. In some embodiments, the non-naturally occurring CD34 peptide ligand comprises an amino acid sequence at least about 80%, 85%, 90%, 95%, 99%, or 100% identical to SEQ ID NO: 68. In some embodiments, the non-naturally occurring CD34 peptide ligand comprises SEQ ID NO: 68. In some embodiments, the non-naturally occurring CD34 peptide ligand comprises an amino acid sequence at least about 80%, 85%, 90%, 95%, 99%, or 100% identical to SEQ ID NO: 69. In some embodiments, the non-naturally occurring CD34 peptide ligand comprises SEQ ID NO: 69. In some embodiments, the non-naturally occurring CD34 peptide ligand comprises an amino acid sequence at least about 80%, 85%, 90%, 95%, 99%, or 100% identical to SEQ ID NO: 70. In some embodiments, the non-naturally occurring CD34 peptide ligand comprises SEQ ID NO: 70. In some embodiments, the non-naturally occurring CD34 peptide ligand comprises an amino acid sequence at least about 80%, 85%, 90%, 95%, 99%, or 100% identical to SEQ ID NO: 71. In some embodiments, the non-naturally occurring CD34 peptide ligand comprises SEQ ID NO: 71. In some embodiments, the non-naturally occurring CD34 peptide ligand comprises an amino acid sequence at least about 80%, 85%, 90%, 95%, 99%, or 100% identical to SEQ ID NO: 72. In some embodiments, the non-naturally occurring CD34 peptide ligand comprises SEQ ID NO: 72. In some embodiments, the non-naturally occurring CD34 peptide ligand comprises an amino acid sequence at least about 80%, 85%, 90%, 95%, 99%, or 100% identical to SEQ ID NO: 73. In some embodiments, the non-naturally occurring CD34 peptide ligand comprises SEQ ID NO: 73. In some embodiments, the non-naturally occurring CD34 peptide ligand comprises an amino acid sequence at least about 80%, 85%, 90%, 95%, 99%, or 100% identical to SEQ ID NO: 74. In some embodiments, the non-naturally occurring CD34 peptide ligand comprises SEQ ID NO: 74. In some embodiments, the non-naturally occurring CD34 peptide ligand comprises an amino acid sequence at least about 80%, 85%, 90%, 95%, 99%, or 100% identical to SEQ ID NO: 75. In some embodiments, the non-naturally occurring CD34 peptide ligand comprises SEQ ID NO: 75. In some embodiments, the non-naturally occurring CD34 peptide ligand comprises an amino acid sequence at least about 80%, 85%, 90%, 95%, 99%, or 100% identical to SEQ ID NO: 76. In some embodiments, the non-naturally occurring CD34 peptide ligand comprises SEQ ID NO: 76. In some embodiments, the non-naturally occurring CD34 peptide ligand comprises an amino acid sequence at least about 80%, 85%, 90%, 95%, 99%, or 100% identical to SEQ ID NO: 77. In some embodiments, the non-naturally occurring CD34 peptide ligand comprises SEQ ID NO: 77. In some embodiments, the non-naturally occurring CD34 peptide ligand comprises an amino acid sequence at least about 80%, 85%, 90%, 95%, 99%, or 100% identical to SEQ ID NO: 78. In some embodiments, the non-naturally occurring CD34 peptide ligand comprises SEQ ID NO: 78. In some embodiments, the non-naturally occurring CD34 peptide ligand comprises an amino acid sequence at least about 80%, 85%, 90%, 95%, 99%, or 100% identical to SEQ ID NO: 79. In some embodiments, the non-naturally occurring CD34 peptide ligand comprises SEQ ID NO: 79. In some embodiments, the non-naturally occurring CD34 peptide ligand comprises an amino acid sequence at least about 80%, 85%, 90%, 95%, 99%, or 100% identical to SEQ ID NO: 80. In some embodiments, the non-naturally occurring CD34 peptide ligand comprises SEQ ID NO: 80. In some embodiments, the non-naturally occurring CD34 peptide ligand comprises an amino acid sequence at least about 80%, 85%, 90%, 95%, 99%, or 100% identical to SEQ ID NO: 81. In some embodiments, the non-naturally occurring CD34 peptide ligand comprises SEQ ID NO: 81. In some embodiments, the non-naturally occurring CD34 peptide ligand comprises an amino acid sequence at least about 80%, 85%, 90%, 95%, 99%, or 100% identical to SEQ ID NO: 82. In some embodiments, the non-naturally occurring CD34 peptide ligand comprises SEQ ID NO: 82. In some embodiments, the non-naturally occurring CD34 peptide ligand comprises an amino acid sequence at least about 80%, 85%, 90%, 95%, 99%, or 100% identical to SEQ ID NO: 83. In some embodiments, the non-naturally occurring CD34 peptide ligand comprises SEQ ID NO: 83. In some embodiments, the non-naturally occurring CD34 peptide ligand comprises an amino acid sequence at least about 80%, 85%, 90%, 95%, 99%, or 100% identical to SEQ ID NO: 84. In some embodiments, the non-naturally occurring CD34 peptide ligand comprises SEQ ID NO: 84. In some embodiments, the non-naturally occurring CD34 peptide ligand comprises an amino acid sequence at least about 80%, 85%, 90%, 95%, 99%, or 100% identical to SEQ ID NO: 85. In some embodiments, the non-naturally occurring CD34 peptide ligand comprises SEQ ID NO: 85.

In some embodiments, the non-naturally occurring CD34 peptide ligand consists of an amino acid sequence at least about 80%, 85%, 90%, 95%, 99%, or 100% identical to a sequence selected from the group consisting of SEQ ID NO: 1-85. In some embodiments, the non-naturally occurring CD34 peptide ligand consists of an amino acid sequence selected from the group consisting of SEQ ID NO: 1-85.

In some embodiments, the non-naturally occurring CD34 peptide ligand consists of an amino acid sequence at least about 80%, 85%, 90%, 95%, 99%, or 100% identical to SEQ ID NO: 1. In some embodiments, the non-naturally occurring CD34 peptide ligand consists of SEQ ID NO: 1. In some embodiments, the non-naturally occurring CD34 peptide ligand consists of an amino acid sequence at least about 80%, 85%, 90%, 95%, 99%, or 100% identical to SEQ ID NO: 2. In some embodiments, the non-naturally occurring CD34 peptide ligand consists of SEQ ID NO: 2. In some embodiments, the non-naturally occurring CD34 peptide ligand consists of an amino acid sequence at least about 80%, 85%, 90%, 95%, 99%, or 100% identical to SEQ ID NO: 3. In some embodiments, the non-naturally occurring CD34 peptide ligand consists of SEQ ID NO: 3. In some embodiments, the non-naturally occurring CD34 peptide ligand consists of an amino acid sequence at least about 80%, 85%, 90%, 95%, 99%, or 100% identical to SEQ ID NO: 4. In some embodiments, the non-naturally occurring CD34 peptide ligand consists of SEQ ID NO: 4. In some embodiments, the non-naturally occurring CD34 peptide ligand consists of an amino acid sequence at least about 80%, 85%, 90%, 95%, 99%, or 100% identical to SEQ ID NO: 5. In some embodiments, the non-naturally occurring CD34 peptide ligand consists of SEQ ID NO: 5. In some embodiments, the non-naturally occurring CD34 peptide ligand consists of an amino acid sequence at least about 80%, 85%, 90%, 95%, 99%, or 100% identical to SEQ ID NO: 6. In some embodiments, the non-naturally occurring CD34 peptide ligand consists of SEQ ID NO: 6. In some embodiments, the non-naturally occurring CD34 peptide ligand consists of an amino acid sequence at least about 80%, 85%, 90%, 95%, 99%, or 100% identical to SEQ ID NO: 7. In some embodiments, the non-naturally occurring CD34 peptide ligand consists of SEQ ID NO: 7. In some embodiments, the non-naturally occurring CD34 peptide ligand consists of an amino acid sequence at least about 80%, 85%, 90%, 95%, 99%, or 100% identical to SEQ ID NO: 8. In some embodiments, the non-naturally occurring CD34 peptide ligand consists of SEQ ID NO: 8. In some embodiments, the non-naturally occurring CD34 peptide ligand consists of an amino acid sequence at least about 80%, 85%, 90%, 95%, 99%, or 100% identical to SEQ ID NO: 9. In some embodiments, the non-naturally occurring CD34 peptide ligand consists of SEQ ID NO: 9. In some embodiments, the non-naturally occurring CD34 peptide ligand consists of an amino acid sequence at least about 80%, 85%, 90%, 95%, 99%, or 100% identical to SEQ ID NO: 10. In some embodiments, the non-naturally occurring CD34 peptide ligand consists of SEQ ID NO: 10. In some embodiments, the non-naturally occurring CD34 peptide ligand consists of an amino acid sequence at least about 80%, 85%, 90%, 95%, 99%, or 100% identical to SEQ ID NO: 11. In some embodiments, the non-naturally occurring CD34 peptide ligand consists of SEQ ID NO: 11. In some embodiments, the non-naturally occurring CD34 peptide ligand consists of an amino acid sequence at least about 80%, 85%, 90%, 95%, 99%, or 100% identical to SEQ ID NO: 12. In some embodiments, the non-naturally occurring CD34 peptide ligand consists of SEQ ID NO: 12. In some embodiments, the non-naturally occurring CD34 peptide ligand consists of an amino acid sequence at least about 80%, 85%, 90%, 95%, 99%, or 100% identical to SEQ ID NO: 13. In some embodiments, the non-naturally occurring CD34 peptide ligand consists of SEQ ID NO: 13. In some embodiments, the non-naturally occurring CD34 peptide ligand consists of an amino acid sequence at least about 80%, 85%, 90%, 95%, 99%, or 100% identical to SEQ ID NO: 14. In some embodiments, the non-naturally occurring CD34 peptide ligand consists of SEQ ID NO: 14. In some embodiments, the non-naturally occurring CD34 peptide ligand consists of an amino acid sequence at least about 80%, 85%, 90%, 95%, 99%, or 100% identical to SEQ ID NO: 15. In some embodiments, the non-naturally occurring CD34 peptide ligand consists of SEQ ID NO: 15. In some embodiments, the non-naturally occurring CD34 peptide ligand consists of an amino acid sequence at least about 80%, 85%, 90%, 95%, 99%, or 100% identical to SEQ ID NO: 16. In some embodiments, the non-naturally occurring CD34 peptide ligand consists of SEQ ID NO: 16. In some embodiments, the non-naturally occurring CD34 peptide ligand consists of an amino acid sequence at least about 80%, 85%, 90%, 95%, 99%, or 100% identical to SEQ ID NO: 17. In some embodiments, the non-naturally occurring CD34 peptide ligand consists of SEQ ID NO: 17. In some embodiments, the non-naturally occurring CD34 peptide ligand consists of an amino acid sequence at least about 80%, 85%, 90%, 95%, 99%, or 100% identical to SEQ ID NO: 18. In some embodiments, the non-naturally occurring CD34 peptide ligand consists of SEQ ID NO: 18. In some embodiments, the non-naturally occurring CD34 peptide ligand consists of an amino acid sequence at least about 80%, 85%, 90%, 95%, 99%, or 100% identical to SEQ ID NO: 19. In some embodiments, the non-naturally occurring CD34 peptide ligand consists of SEQ ID NO: 19. In some embodiments, the non-naturally occurring CD34 peptide ligand consists of an amino acid sequence at least about 80%, 85%, 90%, 95%, 99%, or 100% identical to SEQ ID NO: 20. In some embodiments, the non-naturally occurring CD34 peptide ligand consists of SEQ ID NO: 20. In some embodiments, the non-naturally occurring CD34 peptide ligand consists of an amino acid sequence at least about 80%, 85%, 90%, 95%, 99%, or 100% identical to SEQ ID NO: 21. In some embodiments, the non-naturally occurring CD34 peptide ligand consists of SEQ ID NO: 21. In some embodiments, the non-naturally occurring CD34 peptide ligand consists of an amino acid sequence at least about 80%, 85%, 90%, 95%, 99%, or 100% identical to SEQ ID NO: 22. In some embodiments, the non-naturally occurring CD34 peptide ligand consists of SEQ ID NO: 22. In some embodiments, the non-naturally occurring CD34 peptide ligand consists of an amino acid sequence at least about 80%, 85%, 90%, 95%, 99%, or 100% identical to SEQ ID NO: 23. In some embodiments, the non-naturally occurring CD34 peptide ligand consists of SEQ ID NO: 23. In some embodiments, the non-naturally occurring CD34 peptide ligand consists of an amino acid sequence at least about 80%, 85%, 90%, 95%, 99%, or 100% identical to SEQ ID NO: 24. In some embodiments, the non-naturally occurring CD34 peptide ligand consists of SEQ ID NO: 24. In some embodiments, the non-naturally occurring CD34 peptide ligand consists of an amino acid sequence at least about 80%, 85%, 90%, 95%, 99%, or 100% identical to SEQ ID NO: 25. In some embodiments, the non-naturally occurring CD34 peptide ligand consists of SEQ ID NO: 25. In some embodiments, the non-naturally occurring CD34 peptide ligand consists of an amino acid sequence at least about 80%, 85%, 90%, 95%, 99%, or 100% identical to SEQ ID NO: 26. In some embodiments, the non-naturally occurring CD34 peptide ligand consists of SEQ ID NO: 26. In some embodiments, the non-naturally occurring CD34 peptide ligand consists of an amino acid sequence at least about 80%, 85%, 90%, 95%, 99%, or 100% identical to SEQ ID NO: 27. In some embodiments, the non-naturally occurring CD34 peptide ligand consists of SEQ ID NO: 27. In some embodiments, the non-naturally occurring CD34 peptide ligand consists of an amino acid sequence at least about 80%, 85%, 90%, 95%, 99%, or 100% identical to SEQ ID NO: 28. In some embodiments, the non-naturally occurring CD34 peptide ligand consists of SEQ ID NO: 28. In some embodiments, the non-naturally occurring CD34 peptide ligand consists of an amino acid sequence at least about 80%, 85%, 90%, 95%, 99%, or 100% identical to SEQ ID NO: 29. In some embodiments, the non-naturally occurring CD34 peptide ligand consists of SEQ ID NO: 29. In some embodiments, the non-naturally occurring CD34 peptide ligand consists of an amino acid sequence at least about 80%, 85%, 90%, 95%, 99%, or 100% identical to SEQ ID NO: 30. In some embodiments, the non-naturally occurring CD34 peptide ligand consists of SEQ ID NO: 30. In some embodiments, the non-naturally occurring CD34 peptide ligand consists of an amino acid sequence at least about 80%, 85%, 90%, 95%, 99%, or 100% identical to SEQ ID NO: 31. In some embodiments, the non-naturally occurring CD34 peptide ligand consists of SEQ ID NO: 31. In some embodiments, the non-naturally occurring CD34 peptide ligand consists of an amino acid sequence at least about 80%, 85%, 90%, 95%, 99%, or 100% identical to SEQ ID NO: 32. In some embodiments, the non-naturally occurring CD34 peptide ligand consists of SEQ ID NO: 32. In some embodiments, the non-naturally occurring CD34 peptide ligand consists of an amino acid sequence at least about 80%, 85%, 90%, 95%, 99%, or 100% identical to SEQ ID NO: 33. In some embodiments, the non-naturally occurring CD34 peptide ligand consists of SEQ ID NO: 33. In some embodiments, the non-naturally occurring CD34 peptide ligand consists of an amino acid sequence at least about 80%, 85%, 90%, 95%, 99%, or 100% identical to SEQ ID NO: 34. In some embodiments, the non-naturally occurring CD34 peptide ligand consists of SEQ ID NO: 34. In some embodiments, the non-naturally occurring CD34 peptide ligand consists of an amino acid sequence at least about 80%, 85%, 90%, 95%, 99%, or 100% identical to SEQ ID NO: 35. In some embodiments, the non-naturally occurring CD34 peptide ligand consists of SEQ ID NO: 35. In some embodiments, the non-naturally occurring CD34 peptide ligand consists of an amino acid sequence at least about 80%, 85%, 90%, 95%, 99%, or 100% identical to SEQ ID NO: 36. In some embodiments, the non-naturally occurring CD34 peptide ligand consists of SEQ ID NO: 36. In some embodiments, the non-naturally occurring CD34 peptide ligand consists of an amino acid sequence at least about 80%, 85%, 90%, 95%, 99%, or 100% identical to SEQ ID NO: 37. In some embodiments, the non-naturally occurring CD34 peptide ligand consists of SEQ ID NO: 37. In some embodiments, the non-naturally occurring CD34 peptide ligand consists of an amino acid sequence at least about 80%, 85%, 90%, 95%, 99%, or 100% identical to SEQ ID NO: 38. In some embodiments, the non-naturally occurring CD34 peptide ligand consists of SEQ ID NO: 38. In some embodiments, the non-naturally occurring CD34 peptide ligand consists of an amino acid sequence at least about 80%, 85%, 90%, 95%, 99%, or 100% identical to SEQ ID NO: 39. In some embodiments, the non-naturally occurring CD34 peptide ligand consists of SEQ ID NO: 39. In some embodiments, the non-naturally occurring CD34 peptide ligand consists of an amino acid sequence at least about 80%, 85%, 90%, 95%, 99%, or 100% identical to SEQ ID NO: 40. In some embodiments, the non-naturally occurring CD34 peptide ligand consists of SEQ ID NO: 40. In some embodiments, the non-naturally occurring CD34 peptide ligand consists of an amino acid sequence at least about 80%, 85%, 90%, 95%, 99%, or 100% identical to SEQ ID NO: 41. In some embodiments, the non-naturally occurring CD34 peptide ligand consists of SEQ ID NO: 41. In some embodiments, the non-naturally occurring CD34 peptide ligand consists of an amino acid sequence at least about 80%, 85%, 90%, 95%, 99%, or 100% identical to SEQ ID NO: 42. In some embodiments, the non-naturally occurring CD34 peptide ligand consists of SEQ ID NO: 42. In some embodiments, the non-naturally occurring CD34 peptide ligand consists of an amino acid sequence at least about 80%, 85%, 90%, 95%, 99%, or 100% identical to SEQ ID NO: 43. In some embodiments, the non-naturally occurring CD34 peptide ligand consists of SEQ ID NO: 43. In some embodiments, the non-naturally occurring CD34 peptide ligand consists of an amino acid sequence at least about 80%, 85%, 90%, 95%, 99%, or 100% identical to SEQ ID NO: 44. In some embodiments, the non-naturally occurring CD34 peptide ligand consists of SEQ ID NO: 44. In some embodiments, the non-naturally occurring CD34 peptide ligand consists of an amino acid sequence at least about 80%, 85%, 90%, 95%, 99%, or 100% identical to SEQ ID NO: 45. In some embodiments, the non-naturally occurring CD34 peptide ligand consists of SEQ ID NO: 45. In some embodiments, the non-naturally occurring CD34 peptide ligand consists of an amino acid sequence at least about 80%, 85%, 90%, 95%, 99%, or 100% identical to SEQ ID NO: 46. In some embodiments, the non-naturally occurring CD34 peptide ligand consists of SEQ ID NO: 46. In some embodiments, the non-naturally occurring CD34 peptide ligand consists of an amino acid sequence at least about 80%, 85%, 90%, 95%, 99%, or 100% identical to SEQ ID NO: 47. In some embodiments, the non-naturally occurring CD34 peptide ligand consists of SEQ ID NO: 47. In some embodiments, the non-naturally occurring CD34 peptide ligand consists of an amino acid sequence at least about 80%, 85%, 90%, 95%, 99%, or 100% identical to SEQ ID NO: 48. In some embodiments, the non-naturally occurring CD34 peptide ligand consists of SEQ ID NO: 48. In some embodiments, the non-naturally occurring CD34 peptide ligand consists of an amino acid sequence at least about 80%, 85%, 90%, 95%, 99%, or 100% identical to SEQ ID NO: 49. In some embodiments, the non-naturally occurring CD34 peptide ligand consists of SEQ ID NO: 49. In some embodiments, the non-naturally occurring CD34 peptide ligand consists of an amino acid sequence at least about 80%, 85%, 90%, 95%, 99%, or 100% identical to SEQ ID NO: 50. In some embodiments, the non-naturally occurring CD34 peptide ligand consists of SEQ ID NO: 50. In some embodiments, the non-naturally occurring CD34 peptide ligand consists of an amino acid sequence at least about 80%, 85%, 90%, 95%, 99%, or 100% identical to SEQ ID NO: 51. In some embodiments, the non-naturally occurring CD34 peptide ligand consists of SEQ ID NO: 51. In some embodiments, the non-naturally occurring CD34 peptide ligand consists of an amino acid sequence at least about 80%, 85%, 90%, 95%, 99%, or 100% identical to SEQ ID NO: 52. In some embodiments, the non-naturally occurring CD34 peptide ligand consists of SEQ ID NO: 52. In some embodiments, the non-naturally occurring CD34 peptide ligand consists of an amino acid sequence at least about 80%, 85%, 90%, 95%, 99%, or 100% identical to SEQ ID NO: 53. In some embodiments, the non-naturally occurring CD34 peptide ligand consists of SEQ ID NO: 53. In some embodiments, the non-naturally occurring CD34 peptide ligand consists of an amino acid sequence at least about 80%, 85%, 90%, 95%, 99%, or 100% identical to SEQ ID NO: 54. In some embodiments, the non-naturally occurring CD34 peptide ligand consists of SEQ ID NO: 54. In some embodiments, the non-naturally occurring CD34 peptide ligand consists of an amino acid sequence at least about 80%, 85%, 90%, 95%, 99%, or 100% identical to SEQ ID NO: 55. In some embodiments, the non-naturally occurring CD34 peptide ligand consists of SEQ ID NO: 55. In some embodiments, the non-naturally occurring CD34 peptide ligand consists of an amino acid sequence at least about 80%, 85%, 90%, 95%, 99%, or 100% identical to SEQ ID NO: 56. In some embodiments, the non-naturally occurring CD34 peptide ligand consists of SEQ ID NO: 56. In some embodiments, the non-naturally occurring CD34 peptide ligand consists of an amino acid sequence at least about 80%, 85%, 90%, 95%, 99%, or 100% identical to SEQ ID NO: 57. In some embodiments, the non-naturally occurring CD34 peptide ligand consists of SEQ ID NO: 57. In some embodiments, the non-naturally occurring CD34 peptide ligand consists of an amino acid sequence at least about 80%, 85%, 90%, 95%, 99%, or 100% identical to SEQ ID NO: 58. In some embodiments, the non-naturally occurring CD34 peptide ligand consists of SEQ ID NO: 58. In some embodiments, the non-naturally occurring CD34 peptide ligand consists of an amino acid sequence at least about 80%, 85%, 90%, 95%, 99%, or 100% identical to SEQ ID NO: 59. In some embodiments, the non-naturally occurring CD34 peptide ligand consists of SEQ ID NO: 59. In some embodiments, the non-naturally occurring CD34 peptide ligand consists of an amino acid sequence at least about 80%, 85%, 90%, 95%, 99%, or 100% identical to SEQ ID NO: 60. In some embodiments, the non-naturally occurring CD34 peptide ligand consists of SEQ ID NO: 60. In some embodiments, the non-naturally occurring CD34 peptide ligand consists of an amino acid sequence at least about 80%, 85%, 90%, 95%, 99%, or 100% identical to SEQ ID NO: 61. In some embodiments, the non-naturally occurring CD34 peptide ligand consists of SEQ ID NO: 61. In some embodiments, the non-naturally occurring CD34 peptide ligand consists of an amino acid sequence at least about 80%, 85%, 90%, 95%, 99%, or 100% identical to SEQ ID NO: 62. In some embodiments, the non-naturally occurring CD34 peptide ligand consists of SEQ ID NO: 62. In some embodiments, the non-naturally occurring CD34 peptide ligand consists of an amino acid sequence at least about 80%, 85%, 90%, 95%, 99%, or 100% identical to SEQ ID NO: 63. In some embodiments, the non-naturally occurring CD34 peptide ligand consists of SEQ ID NO: 63. In some embodiments, the non-naturally occurring CD34 peptide ligand consists of an amino acid sequence at least about 80%, 85%, 90%, 95%, 99%, or 100% identical to SEQ ID NO: 64. In some embodiments, the non-naturally occurring CD34 peptide ligand consists of SEQ ID NO: 64. In some embodiments, the non-naturally occurring CD34 peptide ligand consists of an amino acid sequence at least about 80%, 85%, 90%, 95%, 99%, or 100% identical to SEQ ID NO: 65. In some embodiments, the non-naturally occurring CD34 peptide ligand consists of SEQ ID NO: 65. In some embodiments, the non-naturally occurring CD34 peptide ligand consists of an amino acid sequence at least about 80%, 85%, 90%, 95%, 99%, or 100% identical to SEQ ID NO: 66. In some embodiments, the non-naturally occurring CD34 peptide ligand consists of SEQ ID NO: 66. In some embodiments, the non-naturally occurring CD34 peptide ligand consists of an amino acid sequence at least about 80%, 85%, 90%, 95%, 99%, or 100% identical to SEQ ID NO: 67. In some embodiments, the non-naturally occurring CD34 peptide ligand consists of SEQ ID NO: 67. In some embodiments, the non-naturally occurring CD34 peptide ligand consists of an amino acid sequence at least about 80%, 85%, 90%, 95%, 99%, or 100% identical to SEQ ID NO: 68. In some embodiments, the non-naturally occurring CD34 peptide ligand consists of SEQ ID NO: 68. In some embodiments, the non-naturally occurring CD34 peptide ligand consists of an amino acid sequence at least about 80%, 85%, 90%, 95%, 99%, or 100% identical to SEQ ID NO: 69. In some embodiments, the non-naturally occurring CD34 peptide ligand consists of SEQ ID NO: 69. In some embodiments, the non-naturally occurring CD34 peptide ligand consists of an amino acid sequence at least about 80%, 85%, 90%, 95%, 99%, or 100% identical to SEQ ID NO: 70. In some embodiments, the non-naturally occurring CD34 peptide ligand consists of SEQ ID NO: 70. In some embodiments, the non-naturally occurring CD34 peptide ligand consists of an amino acid sequence at least about 80%, 85%, 90%, 95%, 99%, or 100% identical to SEQ ID NO: 71. In some embodiments, the non-naturally occurring CD34 peptide ligand consists of SEQ ID NO: 71. In some embodiments, the non-naturally occurring CD34 peptide ligand consists of an amino acid sequence at least about 80%, 85%, 90%, 95%, 99%, or 100% identical to SEQ ID NO: 72. In some embodiments, the non-naturally occurring CD34 peptide ligand consists of SEQ ID NO: 72. In some embodiments, the non-naturally occurring CD34 peptide ligand consists of an amino acid sequence at least about 80%, 85%, 90%, 95%, 99%, or 100% identical to SEQ ID NO: 73. In some embodiments, the non-naturally occurring CD34 peptide ligand consists of SEQ ID NO: 73. In some embodiments, the non-naturally occurring CD34 peptide ligand consists of an amino acid sequence at least about 80%, 85%, 90%, 95%, 99%, or 100% identical to SEQ ID NO: 74. In some embodiments, the non-naturally occurring CD34 peptide ligand consists of SEQ ID NO: 74. In some embodiments, the non-naturally occurring CD34 peptide ligand consists of an amino acid sequence at least about 80%, 85%, 90%, 95%, 99%, or 100% identical to SEQ ID NO: 75. In some embodiments, the non-naturally occurring CD34 peptide ligand consists of SEQ ID NO: 75. In some embodiments, the non-naturally occurring CD34 peptide ligand consists of an amino acid sequence at least about 80%, 85%, 90%, 95%, 99%, or 100% identical to SEQ ID NO: 76. In some embodiments, the non-naturally occurring CD34 peptide ligand consists of SEQ ID NO: 76. In some embodiments, the non-naturally occurring CD34 peptide ligand consists of an amino acid sequence at least about 80%, 85%, 90%, 95%, 99%, or 100% identical to SEQ ID NO: 77. In some embodiments, the non-naturally occurring CD34 peptide ligand consists of SEQ ID NO: 77. In some embodiments, the non-naturally occurring CD34 peptide ligand consists of an amino acid sequence at least about 80%, 85%, 90%, 95%, 99%, or 100% identical to SEQ ID NO: 78. In some embodiments, the non-naturally occurring CD34 peptide ligand consists of SEQ ID NO: 78. In some embodiments, the non-naturally occurring CD34 peptide ligand consists of an amino acid sequence at least about 80%, 85%, 90%, 95%, 99%, or 100% identical to SEQ ID NO: 79. In some embodiments, the non-naturally occurring CD34 peptide ligand consists of SEQ ID NO: 79. In some embodiments, the non-naturally occurring CD34 peptide ligand consists of an amino acid sequence at least about 80%, 85%, 90%, 95%, 99%, or 100% identical to SEQ ID NO: 80. In some embodiments, the non-naturally occurring CD34 peptide ligand consists of SEQ ID NO: 80. In some embodiments, the non-naturally occurring CD34 peptide ligand consists of an amino acid sequence at least about 80%, 85%, 90%, 95%, 99%, or 100% identical to SEQ ID NO: 81. In some embodiments, the non-naturally occurring CD34 peptide ligand consists of SEQ ID NO: 81. In some embodiments, the non-naturally occurring CD34 peptide ligand consists of an amino acid sequence at least about 80%, 85%, 90%, 95%, 99%, or 100% identical to SEQ ID NO: 82. In some embodiments, the non-naturally occurring CD34 peptide ligand consists of SEQ ID NO: 82. In some embodiments, the non-naturally occurring CD34 peptide ligand consists of an amino acid sequence at least about 80%, 85%, 90%, 95%, 99%, or 100% identical to SEQ ID NO: 83. In some embodiments, the non-naturally occurring CD34 peptide ligand consists of SEQ ID NO: 83. In some embodiments, the non-naturally occurring CD34 peptide ligand consists of an amino acid sequence at least about 80%, 85%, 90%, 95%, 99%, or 100% identical to SEQ ID NO: 84. In some embodiments, the non-naturally occurring CD34 peptide ligand consists of SEQ ID NO: 84. In some embodiments, the non-naturally occurring CD34 peptide ligand consists of an amino acid sequence at least about 80%, 85%, 90%, 95%, 99%, or 100% identical to SEQ ID NO: 85. In some embodiments, the non-naturally occurring CD34 peptide ligand consists of SEQ ID NO: 85.

Percent (%) amino acid sequence “identity” with respect to the sequences identified herein is defined as the percentage of amino acid residues in a candidate sequence that are identical with the amino acid residues in the reference sequence for each of the peptides and/or engineered proteins after aligning the sequences and introducing gaps, if necessary, to achieve the maximum percent sequence identity. Alignment for purposes of determining percent amino acid sequence identity may be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared may be determined. For example, percent amino acid sequence identity values generated using the WU-BLAST-2 computer program uses several search parameters, most of which are set to the default values. Those that are not set to default values (i.e., the adjustable parameters) are set with the following values: overlap span=1, overlap fraction=0.125, word threshold (T)=11 and scoring matrix BLOSUM62.

It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents of the present technology are merely exemplary and that equivalents of such are known in the art. Furthermore, the term “about,” as used herein when referring to a measurable value such as an amount or concentration and the like, is meant to encompass variations of 20%, 10%, 5%, 1%, 0.5%, or even 0.1% of the specified amount.

Also, the disclosure of ranges is intended as a continuous range, including every value between the minimum and maximum values recited, as well as any ranges that may be formed by such values. Also disclosed herein are any and all ratios (and ranges of any such ratios) that may be formed by dividing a disclosed numeric value into any other disclosed numeric value. Accordingly, the skilled person will appreciate that many such ratios, ranges, and ranges of ratios may be unambiguously derived from the numerical values presented herein and in all instances, such ratios, ranges, and ranges of ratios represent various embodiments of the present technology.

### REMARKS

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments can vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

1. A computing device comprising:
a processor;
a first module that, when executed by the processor, is configured to generate multiple peptide sequences based on cell type specificity, tissue specificity, or organ specificity, through the use of a first machine learning model that predicts protein-protein interactions;
a second module that, when executed by the processor, is configured to employ a second machine learning model to predict binding interfaces for the multiple peptide sequences;
a third module that, when executed by the processor, is configured to identify a peptide sequence from among the multiple peptide sequences based on an analysis of the predicted binding interfaces and data related to docking capabilities of the multiple peptide sequences;
a fourth module that, when executed by the processor, is configured to enhance one or more properties of a peptide represented by the peptide sequence through simulated mutagenesis of the peptide sequence; and
a fifth module that, when executed by the processor, is configured to generate instructions for instrumentation that is able to synthesize the mutated peptide sequence of the peptide.

2. The computing device of claim 1, wherein the second machine learning model is based on a neural network that implements a reinforcement learning algorithm.

3. The computing device of claim 1, wherein the binding interfaces are predicted between a series of ligands and a series of biological targets.

4. The computing device of claim 1, further comprising:
a communication module that is configured to provide access to one or more databases containing data relating to proteins, cells, tissues, organs, structures, surfactomics, or proteomics.

5. The computing device of claim 1, further comprising:
a sixth module that, when executed by the processor, is configured to generate visualizations that include information regarding the peptide sequence, so as to facilitate informed decision making with respect to development and synthesis of the peptide.

6. A method for developing a peptide having a therapeutic application, the method comprising:
receiving input that is indicative of a selection of an organ, a tissue, or a cell type;
generating, based on the input, multiple amino acid sequences that are representative of multiple peptides;
predicting binding interfaces for each of the multiple peptides;
identifying, based on the binding interfaces, a given peptide from among the multiple peptides;
enhancing a property of the given peptide through simulated single-point mutagenesis across an interacting surface of the given peptide; and
documenting the given peptide, with the enhanced property, by storing information in a data structure.

7. The method of claim 6, wherein said generating comprises:
identifying a dataset that includes information regarding the selected organ, tissue, or cell type, and
applying, to the dataset, a machine learning model that predicts protein-protein interactions for each of the different peptides.

8. The method of claim 7, wherein the machine learning model is trained on another dataset that includes information regarding known protein-protein interactions determined through x-ray crystallography data or cryo-electron microscopy data.

9. The method of claim 6, further comprising:
transmitting the data structure to instrumentation to prompt synthesis of the given peptide.

10. The method of claim 6, wherein the property is energetics, solubility, binding affinity, or delivery mechanism.

11. A non-naturally occurring peptide ligand of CD34 comprising or consisting of an amino acid sequence at least about 80%, 85%, 90%, 95%, 99%, or 100% identical to a sequence selected from the group consisting of SEQ ID NO: 1-85.

12. The non-naturally occurring peptide ligand of CD34 of claim 11, wherein the non-naturally occurring peptide ligand of CD34 comprises or consists of an amino acid sequence selected from the group consisting of SEQ ID NO: 1-85.

13-182. (canceled)
